(19) United States
(12) Patent Application Publication
(10) Pub. No.: US 2026/0014194 A1
(43) Pub. Date: Jan. 15, 2026

(54) COMBINATIONS COMPRISING METAP2 INHIBITORS FOR THE TREATMENT OF CANCER

(71) Applicant: SynDevRx, Inc., Cambridge, MA (US)

(72) Inventors: Bradley J. CARVER, Cambridge, MA (US); James SHANAHAN, Cambridge, MA (US); Peter CORNELIUS, Framingham, MA (US); Benjamin MAYES, Cambridge, MA (US)

(21) Appl. No.: 18/846,365

(22) PCT Filed: Mar. 16, 2023
(86) PCT No.: PCT/US2023/064550

§ 371 Date: Sep. 12, 2024

Related U.S. Application Data
(60) Provisional application No. 63/320,347, filed on Mar. 16, 2022.
(60) Provisional application No. 63/340,726, filed on May 11, 2022.

(51) Int. Cl. A61K 31/785; A61K 31/357; A61K 31/4439; A61K 31/553; A61K 31/565; A61P 35/00

(52) U.S. Cl. CPC A61K 31/785; A61K 31/357; A61K 31/4439; A61K 31/553; A61K 31/565; A61P 35/00

(57) ABSTRACT

The present disclosure provides pharmaceutical combinations comprising MetAP2 inhibitors for the treatment of cancer.

### CROSS REFERENCE TO RELATED APPLICATIONS

This application claims priority to, and the benefit of, U.S. Provisional Application No. 63/320,347, filed on Mar. 16, 2022, and U.S. Provisional Application No. 63/340,726, filed on May 11, 2022. The contents of each of the aforementioned patent applications are incorporated herein by reference in their entireties.

### BACKGROUND

Breast cancer is the most common cancer diagnosed among US women and is the second leading cause of cancer-related deaths. Despite advances in breast cancer therapeutics, specific subtypes of breast cancer and specific subpopulations of breast cancer patients are still recalcitrant to treatment and exhibit higher mortalities.

For example, triple-negative breast cancer (TNBC), which is 1 of the 4 clinically categorized subtypes of invasive breast cancer and account for 10-20% of total breast cancer cases, exhibits higher aggressiveness than other breast cancer subtypes. TNBC exhibits a negative phenotype for the estrogen receptor (ER) and progesterone receptor (PR) and a lack of gene amplification/protein overexpression for the human epidermal growth factor receptor 2 (HER2). These specific characteristics of TNBC means that patients with TNBC do not benefit from hormonal therapy or treatments targeting the oncogenic HER2 pathway.

Treatment of TNBC patients with recurrent and/or metastatic disease instead relies on cytotoxic chemotherapy, with a median survival of approximately 13 months from the time of recurrence or diagnosis of distant metastases. Accordingly, there is an urgent need in the art for improved compositions and methods for the treatment of TNBC.

In another example, many instances of breast cancer exhibit mutations in the PIK3CA gene. Breast cancer with these PIK3CA gene mutations are often more aggressive, leading to increased mortality rates. While several therapeutics specifically targeting the PIK3CA gene product PI3Kα either specifically or as part of a pan-PI3K isoform targeting treatment (e.g., copanlisib) demonstrated clinical benefit in patients with PIK3CA mutations, these therapeutics induce dangerous side-effects such as hyperglycemia and hyperinsulinemia that limits these drugs effectiveness in the clinic. Accordingly, there is an urgent need in the art for improved compositions and methods for the treatment of breast cancers with a PIK3CA gene mutation, including compositions and methods which alleviate the dangerous side-effects of existing PI3Kα inhibitors.

The present disclosure provides combinations comprising MetAP2 inhibitors for the treatment of breast cancer.

### SUMMARY

The present disclosure provides a combination comprising at least one MetAP2 inhibitor, or a pharmaceutically acceptable salt thereof, and eribulin, or a pharmaceutically acceptable salt thereof, for use in treating a cancer in a subject.

The present disclosure provides a method of treating cancer in a subject in need thereof, the method comprising administering to the subject at least one therapeutically effective amount of at least one MetAP2 inhibitor, or a pharmaceutically acceptable salt thereof, and at least one therapeutically effective amount of eribulin, or a pharmaceutically acceptable salt thereof.

The present disclosure provides a MetAP2 inhibitor, or a pharmaceutically acceptable salt thereof, for use in a method of treating a cancer in a subject, wherein the method further comprises administration of eribulin, or a pharmaceutically acceptable salt thereof.

The present disclosure provides eribulin, or a pharmaceutically acceptable salt thereof, for use in a method of treating a cancer in a subject, wherein the method further comprises administration of at least one MetAP2 inhibitor or a pharmaceutically acceptable salt thereof.

The present disclosure provides a combination comprising at least one MetAP2 inhibitor, or a pharmaceutically acceptable salt thereof, fulvestrant, or a pharmaceutically acceptable salt thereof, and alpelisib, or a pharmaceutically acceptable salt thereof, for use in treating a cancer in a subject.

The present disclosure provides a method of treating cancer in a subject in need thereof, the method comprising administering to the subject at least one therapeutically effective amount of at least one MetAP2 inhibitor, or a pharmaceutically acceptable salt thereof, at least one therapeutically effective amount of fulvestrant, or a pharmaceutically acceptable salt thereof, and at least one therapeutically effective amount of alpelisib, or a pharmaceutically acceptable salt thereof.

The present disclosure provides a MetAP2 inhibitor, or a pharmaceutically acceptable salt thereof, for use in a method of treating a cancer in a subject, wherein the method further comprises administration of fulvestrant, or a pharmaceutically acceptable salt thereof, and alpelisib, or a pharmaceutically acceptable salt thereof.

The present disclosure provides fulvestrant, or a pharmaceutically acceptable salt thereof, for use in a method of treating a cancer in a subject, wherein the method further comprises administration of at least one MetAP2 inhibitor, or a pharmaceutically acceptable salt thereof, and alpelisib, or a pharmaceutically acceptable salt thereof.

The present disclosure provides alpelisib, or a pharmaceutically acceptable salt thereof, for use in a method of treating a cancer in a subject, wherein the method further comprises administration of at least one MetAP2 inhibitor, or a pharmaceutically acceptable salt thereof, and fulvestrant, or a pharmaceutically acceptable salt thereof.

Any of the above aspects, or any other aspect described herein, can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is a schematic of a dosing schedule of the present disclosure.

FIG. 2 is a schematic of a dosing schedule of the present disclosure.

FIG. 3 is a graph of tumor volume in mice treated with various combinations of the present disclosure.

FIG. 4 is a graph of tumor volume in individual animals treated with either vehicle control or eribulin at 2/mg/kg.

FIG. 5 is a series of graphs showing tumor volume in mice treated with various combinations of the present disclosure.

FIG. 6 is a series of graphs showing tumor volume in mice treated with various combinations of the present disclosure.

FIG. 7 is a series of graphs showing tumor volume in mice treated with various combinations of the present disclosure.

FIG. 8 is a series of charts showing tumor growth inhibition in mice treated with various combinations of the present disclosure.

FIG. 9 is a series of graphs showing body weight and change in body weight in in mice treated with various combinations of the present disclosure.

FIG. 10 is a graph showing percentage survival in mice treated with various combinations of the present disclosure.

FIG. 11 is a series of graphs showing percentage survival in mice treated with various combinations of the present disclosure.

FIG. 12 is a graph showing the adipose tissue mass measured in mice treated with various combinations of the present disclosure.

FIG. 13 is a graph showing leptin levels in mice treated with various combinations of the present disclosure.

FIG. 14 is a graph showing plasma adiponectin levels in mice treated with various combinations of the present disclosure.

FIG. 15 is a graph showing the leptin/adiponectin ration (LAR) in the plasma samples isolated from mice treated with various combinations of the present disclosure.

FIG. 16 is a graph showing plasma insulin levels in mice treated with various combinations of the present disclosure.

FIG. 17 is a graph showing plasma SFRP1 levels in mice treated with various combinations of the present disclosure.

FIG. 18 is a series of graphs showing plasma interleukin levels in mice treated with various combinations of the present disclosure.

FIG. 19 is a series of graphs showing plasma hematopoietic growth factor levels (G-CSF and M-CSF) in the plasma samples isolated from mice treated with various combinations of the present disclosure.

FIG. 20 is a series of graphs showing the levels of FABP4 and Resistin in plasma samples isolated from mice treated with various combinations of the present disclosure.

FIG. 21 is a graph showing plasma FGF-21 levels in mice treated with various combinations of the present disclosure.

FIG. 22 is a series of graphs showing ALP, ALT and AST levels in mice treated with various combinations of the present disclosure.

FIG. 23 is a graph showing cholesterol levels in mice treated with various combinations of the present disclosure.

FIG. 24 is a graph showing bilirubin levels in the various treatment groups.

FIG. 25 is a graph showing creatine kinase levels in mice treated with various combinations of the present disclosure.

FIG. 26 is a graph showing albumin levels in mice treated with various combinations of the present disclosure.

FIG. 27 is a graph showing globulin levels in mice treated with various combinations of the present disclosure.

FIG. 28 is a graph showing albumin to globulin ratio (AGR) in mice treated with various combinations of the present disclosure.

FIG. 29 is a series of graphs showing red blood cell (RBC) and hematocrit (HCT) levels in mice treated with various combinations of the present disclosure.

FIG. 30 is a graph showing hemoglobin (Hgb) levels in the various treatment groups.

FIG. 31 is a series of graphs showing white blood cell (WBC) and monocyte levels in mice treated with various combinations of the present disclosure.

FIG. 32 is a series of graphs showing lymphocyte and neutrophil levels in mice treated with various combinations of the present disclosure.

FIG. 33 is a series of graphs showing tumor volume and change in tumor volume in mice treated with various combinations of the present disclosure.

FIG. 34 is a series of graphs showing body weight and change in body weight in in mice treated with various combinations of the present disclosure.

### DETAILED DESCRIPTION

The present disclosure provides, inter alia, a method of treating cancer or preventing treatment-induced hyperglycemia, comprising administering to a subject in need thereof at least one therapeutically effective amount of at least one MetAP2 inhibitor of the present disclosure, or a pharmaceutically acceptable salt thereof, in combination with at least one additional therapeutic agent (e.g. a second therapeutic agent, or a second and a third therapeutic agent).

### Combinations and Methods of the Present Disclosure

The present disclosure provides a combination therapy comprising at least one therapeutically effective amount of at least one MetAP2 inhibitor of the present disclosure, or a pharmaceutically acceptable salt thereof, at least one therapeutically effective amount of at least one PI3K inhibitor, or a pharmaceutically acceptable salt thereof, and at least one therapeutically effective amount of at least one selective estrogen receptor degrader (SERD).

The present disclosure provides a method of treating cancer in a subject in need thereof, the method comprising administering to the subject in need thereof at least one therapeutically effective amount of the preceding combination therapy.

The present disclosure provides a method of preventing and/or mitigating treatment-induced hyperglycemia in a subject in need thereof, the method comprising administering to the subject in need thereof at least one therapeutically effective amount of the preceding combination therapy.

The present disclosure provides a pharmaceutical composition comprising at least one therapeutically effective amount of at least one MetAP2 inhibitor of the present disclosure, or a pharmaceutically acceptable salt thereof, at least one therapeutically effective amount of at least one PI3K inhibitor, or a pharmaceutically acceptable salt thereof, and at least one therapeutically effective amount of at least one selective estrogen receptor degrader (SERD).

The present disclosure provides a method of treating cancer in a subject in need thereof, the method comprising administering to the subject in need thereof at least one therapeutically effective amount of the preceding pharmaceutical composition.

The present disclosure provides a method of preventing and/or mitigating treatment-induced hyperglycemia in a subject in need thereof, the method comprising administering to the subject in need thereof at least one therapeutically effective amount of the preceding pharmaceutical composition.

The present disclosure provides a kit comprising at least one therapeutically effective amount of at least one MetAP2 inhibitor of the present disclosure, or a pharmaceutically acceptable salt thereof at least one therapeutically effective amount of at least one PI3K inhibitor, or a pharmaceutically acceptable salt thereof, and at least one therapeutically effective amount of at least one selective estrogen receptor degrader (SERD).

The present disclosure provides a method of treating cancer in a subject in need thereof, the method comprising administering to the subject in need thereof at least one therapeutically effective amount of the preceding kit.

The present disclosure provides a method of preventing and/or mitigating treatment-induced hyperglycemia in a subject in need thereof, the method comprising administering to the subject at least one therapeutically effective amount of the preceding kit.

The present disclosure provides a method of treating cancer in a subject in need thereof, the method comprising administering to the subject at least one therapeutically effective amount of at least one MetAP2 inhibitor of the present disclosure, or a pharmaceutically acceptable salt thereof, at least one therapeutically effective amount of at least one PI3K inhibitor, or a pharmaceutically acceptable salt thereof, and at least one therapeutically effective amount of at least one selective estrogen receptor degrader (SERD).

The present disclosure provides a method of preventing and/or mitigating treatment-induced hyperglycemia in a subject in need thereof, the method comprising administering to the subject at least one therapeutically effective amount of at least one MetAP2 inhibitor of the present disclosure, or a pharmaceutically acceptable salt thereof, at least one therapeutically effective amount of at least one PI3K inhibitor, or a pharmaceutically acceptable salt thereof, and at least one therapeutically effective amount of at least one selective estrogen receptor degrader (SERD).

The present disclosure provides a use of at least one MetAP2 inhibitor of the present disclosure, or a pharmaceutically acceptable salt thereof, in combination with at least one PI3K inhibitor, or a pharmaceutically acceptable salt thereof, and at least one SERD, or a pharmaceutically acceptable salt thereof, in the manufacture of a medicament for the treatment of a cancer.

The present disclosure provides a use of at least one MetAP2 inhibitor of the present disclosure, or a pharmaceutically acceptable salt thereof, in combination with at least one PI3K inhibitor, or a pharmaceutically acceptable salt thereof, and at least one SERD, or a pharmaceutically acceptable salt thereof, in the manufacture of a medicament for the prevention and/or the mitigation of treatment-induced hyperglycemia in a subject in need thereof.

The present disclosure provides a use of at least one PI3K inhibitor, or a pharmaceutically acceptable salt thereof, in combination with at least one MetAP2 inhibitor of the present disclosure, or a pharmaceutically acceptable salt thereof, and at least one SERD, or pharmaceutically acceptable salt thereof in the manufacture of a medicament for the treatment of a cancer.

The present disclosure provides a use of at least one PI3K inhibitor, or a pharmaceutically acceptable salt thereof, in combination with at least one MetAP2 inhibitor of the present disclosure, or a pharmaceutically acceptable salt thereof, and at least one SERD, or pharmaceutically acceptable salt thereof in the manufacture of a medicament for the prevention and/or the mitigation of treatment-induced hyperglycemia in a subject in need thereof.

The present disclosure provides a combination of at least one MetAP2 inhibitor of the present disclosure, or a pharmaceutically acceptable salt thereof, at least one PI3K inhibitor, or a pharmaceutically acceptable salt thereof, and at least one SERD, or a pharmaceutically acceptable salt thereof, for use in the manufacture of a medicament for the treatment of a cancer.

The present disclosure provides a combination of at least one MetAP2 inhibitor of the present disclosure, or a pharmaceutically acceptable salt thereof, at least one PI3K inhibitor, or a pharmaceutically acceptable salt thereof, and at least one SERD, or a pharmaceutically acceptable salt thereof, for use in the manufacture of a medicament for the prevention and/or the mitigation of treatment-induced hyperglycemia in a subject in need thereof.

The present disclosure provides at least one MetAP2 inhibitor of the present disclosure, or a pharmaceutically acceptable salt thereof, for use in combination with at least one PI3K inhibitor or a pharmaceutically acceptable salt thereof, and at least one SERD, or a pharmaceutically acceptable salt thereof, in treating a cancer.

The present disclosure provides at least one MetAP2 inhibitor of the present disclosure, or a pharmaceutically acceptable salt thereof, for use in combination with at least one PI3K inhibitor or a pharmaceutically acceptable salt thereof, and at least one SERD, or a pharmaceutically acceptable salt thereof, in preventing and/or mitigating treatment-induced hyperglycemia in a subject in need thereof.

The present disclosure provides at least one PI3K inhibitor or a pharmaceutically acceptable salt thereof, for use in combination with at least one MetAP2 inhibitor of the present disclosure, or a pharmaceutically acceptable salt thereof, and at least one SERD, or a pharmaceutically acceptable salt thereof, in treating a cancer.

The present disclosure provides at least one PI3K inhibitor or a pharmaceutically acceptable salt thereof, for use in combination with at least one MetAP2 inhibitor of the present disclosure, or a pharmaceutically acceptable salt thereof, and at least one SERD, or a pharmaceutically acceptable salt thereof, in preventing and/or mitigating treatment-induced hyperglycemia in a subject in need thereof.

The present disclosure provides a combination of at least one MetAP2 inhibitor of the present disclosure, or a pharmaceutically acceptable salt thereof, at least one PI3K inhibitor or a pharmaceutically acceptable salt thereof, and at least one SERD, or a pharmaceutically acceptable salt thereof, for use in treating a cancer.

The present disclosure provides a combination of at least one MetAP2 inhibitor of the present disclosure, or a pharmaceutically acceptable salt thereof, at least one PI3K inhibitor or a pharmaceutically acceptable salt thereof, and at least one SERD, or a pharmaceutically acceptable salt thereof, for use in preventing and/or mitigating treatment-induced hyperglycemia in a subject in need thereof.

The present disclosure provides a combination comprising at least one MetAP2 inhibitor of the present disclosure, or a pharmaceutically acceptable salt thereof, at least one PI3K inhibitor or a pharmaceutically acceptable salt thereof, and at least one SERD, or a pharmaceutically acceptable salt thereof, for use in treating a cancer. The present disclosure provides a combination comprising at least one MetAP2 inhibitor of the present disclosure, or a pharmaceutically acceptable salt thereof, for use in treating a cancer, wherein the combination further comprises at least one PI3K inhibitor, or a pharmaceutically acceptable salt thereof and at least one SERD, or a pharmaceutically acceptable salt thereof. The present disclosure provides a combination comprising at least one PI3K inhibitor, or a pharmaceutically acceptable salt thereof, for use in treating a cancer, wherein the combination further comprises at least one MetAP2 inhibitor of the present disclosure, or a pharmaceutically acceptable salt thereof, and at least one SERD, or a pharmaceutically acceptable salt thereof. The present disclosure provides a combination comprising at least one SERD, or a pharmaceutically acceptable salt thereof, for use in treating a cancer, wherein the combination further comprises at least one MetAP2 inhibitor of the present disclosure, or a pharmaceutically acceptable salt thereof, and at least one PI3K inhibitor, or a pharmaceutically acceptable salt thereof.

A MetAP2 inhibitor, or a pharmaceutically acceptable salt thereof, for use in a method of treating a cancer, wherein the method further comprises administration of at least one PI3K inhibitor, or a pharmaceutically acceptable salt thereof, and at least one SERD, or a pharmaceutically acceptable salt thereof.

A PI3K inhibitor, or a pharmaceutically acceptable salt thereof, for use in a method of treating a cancer, wherein the method further comprises administration of at least one MetAP2 inhibitor, or a pharmaceutically acceptable salt thereof, and at least one SERD, or a pharmaceutically acceptable salt thereof.

A SERD, or a pharmaceutically acceptable salt thereof, for use in a method of treating a cancer, wherein the method further comprises administration of at least one PI3K inhibitor, or a pharmaceutically acceptable salt thereof, and at least one MetAP2 inhibitor, or a pharmaceutically acceptable salt thereof.

In some aspects, a MetAP2 inhibitor, or pharmaceutically acceptable salt thereof, a PI3K inhibitor, or pharmaceutically acceptable salt thereof, and a SERD, or a pharmaceutically acceptable salt thereof, can be administered by the same administration route. In some aspects, a MetAP2 inhibitor, or pharmaceutically acceptable salt thereof, a PI3K inhibitor, or pharmaceutically acceptable salt thereof, and a SERD, or a pharmaceutically acceptable salt thereof, can be administered by different administration routes. In some aspects, two of the three compounds in the triplet combination can be administered by the same administration route and the other compound in the combination can be administered by a different administration route.

In some aspects, the MetAP2 inhibitor, or pharmaceutically acceptable salt thereof, the PI3K inhibitor, or pharmaceutically acceptable salt thereof, and the SERD, or a pharmaceutically acceptable salt thereof can be administered concurrently. In some aspects, two of the three compounds in the triplet combination can be administered concurrently.

In some aspects, the MetAP2 inhibitor, or pharmaceutically acceptable salt thereof, the PI3K inhibitor, or pharmaceutically acceptable salt thereof, and the SERD, or a pharmaceutically acceptable salt thereof, can be administered in temporal proximity.

In some aspects, the MetAP2 inhibitor, or pharmaceutically acceptable salt thereof, the PI3K inhibitor, or pharmaceutically acceptable salt thereof, and the SERD, or a pharmaceutically acceptable salt thereof can be administered in any order.

The present disclosure provides a combination therapy comprising at least one therapeutically effective amount of at least one MetAP2 inhibitor of the present disclosure, or a pharmaceutically acceptable salt thereof, and at least one therapeutically effective amount of at least one microtubule-targeting compound, or a pharmaceutically acceptable salt thereof.

The present disclosure provides a method of treating cancer in a subject in need thereof, the method comprising administering to the subject in need thereof at least one therapeutically effective amount of the preceding combination therapy.

The present disclosure provides a pharmaceutical composition comprising at least one therapeutically effective amount of at least one MetAP2 inhibitor of the present disclosure, or a pharmaceutically acceptable salt thereof, and at least one therapeutically effective amount of at least one microtubule-targeting compound, or a pharmaceutically acceptable salt thereof.

The present disclosure provides a method of treating cancer in a subject in need thereof, the method comprising administering to the subject in need thereof at least one therapeutically effective amount of the preceding pharmaceutical composition.

The present disclosure provides a kit comprising at least one therapeutically effective amount of at least one MetAP2 inhibitor of the present disclosure, or a pharmaceutically acceptable salt thereof, and at least one therapeutically effective amount of at least one microtubule-targeting compound, or a pharmaceutically acceptable salt thereof.

The present disclosure provides a method of treating cancer in a subject in need thereof, the method comprising administering to the subject in need thereof at least one therapeutically effective amount of the preceding kit.

The present disclosure provides a method of treating cancer in a subject in need thereof, the method comprising administering to the subject at least one therapeutically effective amount of at least one MetAP2 inhibitor of the present disclosure, or a pharmaceutically acceptable salt thereof, and at least one therapeutically effective amount of at least one microtubule-targeting compound, or a pharmaceutically acceptable salt thereof.

The present disclosure provides a use of at least one MetAP2 inhibitor of the present disclosure, or a pharmaceutically acceptable salt thereof, in combination with at least one microtubule-targeting compound, or a pharmaceutically acceptable salt thereof, in the manufacture of a medicament for the treatment of a cancer.

The present disclosure provides a use of at least one microtubule-targeting compound, or a pharmaceutically acceptable salt thereof, in combination with at least one MetAP2 inhibitor of the present disclosure, or a pharmaceutically acceptable salt thereof, in the manufacture of a medicament for the treatment of a cancer.

The present disclosure provides a combination of at least one MetAP2 inhibitor of the present disclosure, or a pharmaceutically acceptable salt thereof and at least one microtubule-targeting compound, or a pharmaceutically acceptable salt thereof for use in the manufacture of a medicament for the treatment of a cancer.

The present disclosure provides at least one MetAP2 inhibitor of the present disclosure, or a pharmaceutically acceptable salt thereof, for use in combination with at least one microtubule-targeting compound, or a pharmaceutically acceptable salt thereof.

The present disclosure provides at least one microtubule-targeting compound, or a pharmaceutically acceptable salt thereof, for use in combination with at least one MetAP2 inhibitor of the present disclosure, or a pharmaceutically acceptable salt thereof, in treating a cancer.

The present disclosure provides a combination of at least one MetAP2 inhibitor of the present disclosure, or a pharmaceutically acceptable salt thereof, and at least one microtubule-targeting compound or a pharmaceutically acceptable salt thereof, for use in treating a cancer.

The present disclosure provides a combination comprising at least one MetAP2 inhibitor of the present disclosure, or a pharmaceutically acceptable salt thereof, and at least one microtubule-targeting compound, or a pharmaceutically acceptable salt thereof for use in treating a cancer. The present disclosure provides a combination comprising at least one MetAP2 inhibitor of the present disclosure, or a pharmaceutically acceptable salt thereof, for use in treating a cancer, wherein the combination further comprises at least one microtubule-targeting compound, or a pharmaceutically acceptable salt thereof. The present disclosure provides a combination comprising at least one microtubule-targeting compound, or a pharmaceutically acceptable salt thereof, for use in treating a cancer, wherein the combination further comprises at least one MetAP2 inhibitor of the present disclosure, or a pharmaceutically acceptable salt thereof.

A MetAP2 inhibitor, or a pharmaceutically acceptable salt thereof, for use in a method of treating a cancer, wherein the method further comprises administration of at least one microtubule-targeting compound, or a pharmaceutically acceptable salt thereof.

A microtubule-targeting compound, or a pharmaceutically acceptable salt thereof, for use in a method of treating a cancer, wherein the method further comprises administration of at least one MetAP2 inhibitor, or a pharmaceutically acceptable salt thereof.

In some aspects, a MetAP2 inhibitor, or pharmaceutically acceptable salt thereof, and a microtubule-targeting compound, or pharmaceutically acceptable salt thereof, can be administered by the same administration route. In some aspects, a MetAP2 inhibitor, or pharmaceutically acceptable salt thereof, and a microtubule-targeting compound, or pharmaceutically acceptable salt thereof, can be administered by different administration routes.

In some aspects, the MetAP2 inhibitor, or pharmaceutically acceptable salt thereof, and the microtubule-targeting compound, or pharmaceutically acceptable salt thereof, can be administered concurrently.

In some aspects, the MetAP2 inhibitor, or pharmaceutically acceptable salt thereof, and the microtubule-targeting compound, or pharmaceutically acceptable salt thereof, can be administered in temporal proximity.

In some aspects, the MetAP2 inhibitor, or pharmaceutically acceptable salt thereof, and the microtubule-targeting compound, or pharmaceutically acceptable salt thereof, can be administered in any order.

The present disclosure provides a combination therapy comprising at least one therapeutically effective amount of at least one MetAP2 inhibitor of the present disclosure, or a pharmaceutically acceptable salt thereof, and at least one therapeutically effective amount of inavolisib (GDC-0077), or a pharmaceutically acceptable salt thereof.

The present disclosure provides a method of treating cancer in a subject in need thereof, the method comprising administering to the subject in need thereof at least one therapeutically effective amount of the preceding combination therapy.

The present disclosure provides a method of preventing and/or mitigating treatment-induced hyperglycemia in a subject in need thereof, the method comprising administering to the subject in need thereof at least one therapeutically effective amount of the preceding combination therapy.

The present disclosure provides a pharmaceutical composition comprising at least one therapeutically effective amount of at least one MetAP2 inhibitor of the present disclosure, or a pharmaceutically acceptable salt thereof, and at least one therapeutically effective amount of inavolisib (GDC-0077), or a pharmaceutically acceptable salt thereof.

The present disclosure provides a method of treating cancer in a subject in need thereof, the method comprising administering to the subject in need thereof at least one therapeutically effective amount of the preceding pharmaceutical composition.

The present disclosure provides a method of preventing and/or mitigating treatment-induced hyperglycemia in a subject in need thereof, the method comprising administering to the subject in need thereof at least one therapeutically effective amount of the preceding pharmaceutical composition.

The present disclosure provides a kit comprising at least one therapeutically effective amount of at least one MetAP2 inhibitor of the present disclosure, or a pharmaceutically acceptable salt thereof, and at least one therapeutically effective amount of inavolisib (GDC-0077), or a pharmaceutically acceptable salt thereof.

The present disclosure provides a method of treating cancer in a subject in need thereof, the method comprising administering to the subject in need thereof at least one therapeutically effective amount of the preceding kit.

The present disclosure provides a method of preventing and/or mitigating treatment-induced hyperglycemia in a subject in need thereof, the method comprising administering to the subject at least one therapeutically effective amount of the preceding kit.

The present disclosure provides a method of treating cancer in a subject in need thereof, the method comprising administering to the subject at least one therapeutically effective amount of at least one MetAP2 inhibitor of the present disclosure, or a pharmaceutically acceptable salt thereof, and at least one therapeutically effective amount of inavolisib (GDC-0077), or a pharmaceutically acceptable salt thereof.

The present disclosure provides a use of at least one MetAP2 inhibitor of the present disclosure, or a pharmaceutically acceptable salt thereof, in combination with inavolisib (GDC-0077), or a pharmaceutically acceptable salt thereof, in the manufacture of a medicament for the treatment of a cancer.

The present disclosure provides a use of inavolisib (GDC-0077), or a pharmaceutically acceptable salt thereof, in combination with at least one MetAP2 inhibitor of the present disclosure, or a pharmaceutically acceptable salt thereof, in the manufacture of a medicament for the treatment of a cancer.

The present disclosure provides a combination of at least one MetAP2 inhibitor of the present disclosure, or a pharmaceutically acceptable salt thereof and inavolisib (GDC-0077), or a pharmaceutically acceptable salt thereof for use in the manufacture of a medicament for the treatment of a cancer.

The present disclosure provides at least one MetAP2 inhibitor of the present disclosure, or a pharmaceutically acceptable salt thereof, for use in combination with inavolisib (GDC-0077), or a pharmaceutically acceptable salt thereof.

The present disclosure provides inavolisib (GDC-0077), or a pharmaceutically acceptable salt thereof, for use in combination with at least one MetAP2 inhibitor of the present disclosure, or a pharmaceutically acceptable salt thereof, in treating a cancer.

The present disclosure provides a combination of at least one MetAP2 inhibitor of the present disclosure, or a pharmaceutically acceptable salt thereof, and inavolisib (GDC-0077), or a pharmaceutically acceptable salt thereof, for use in treating a cancer.

The present disclosure provides a combination comprising at least one MetAP2 inhibitor of the present disclosure, or a pharmaceutically acceptable salt thereof, and inavolisib (GDC-0077), or a pharmaceutically acceptable salt thereof for use in treating a cancer. The present disclosure provides a combination comprising at least one MetAP2 inhibitor of the present disclosure, or a pharmaceutically acceptable salt thereof, for use in treating a cancer, wherein the combination further comprises inavolisib (GDC-0077), or a pharmaceutically acceptable salt thereof. The present disclosure provides a combination comprising inavolisib (GDC-0077), or a pharmaceutically acceptable salt thereof, for use in treating a cancer, wherein the combination further comprises at least one MetAP2 inhibitor of the present disclosure, or a pharmaceutically acceptable salt thereof.

A MetAP2 inhibitor, or a pharmaceutically acceptable salt thereof, for use in a method of treating a cancer, wherein the method further comprises administration of inavolisib (GDC-0077), or a pharmaceutically acceptable salt thereof.

Inavolisib (GDC-0077), or a pharmaceutically acceptable salt thereof, for use in a method of treating a cancer, wherein the method further comprises administration of at least one MetAP2 inhibitor, or a pharmaceutically acceptable salt thereof.

In some aspects, a MetAP2 inhibitor, or pharmaceutically acceptable salt thereof, and inavolisib (GDC-0077), or pharmaceutically acceptable salt thereof, can be administered by the same administration route. In some aspects, a MetAP2 inhibitor, or pharmaceutically acceptable salt thereof, and inavolisib (GDC-0077), or pharmaceutically acceptable salt thereof, can be administered by different administration routes.

In some aspects, the MetAP2 inhibitor, or pharmaceutically acceptable salt thereof, and inavolisib (GDC-0077), or pharmaceutically acceptable salt thereof, can be administered concurrently.

In some aspects, the MetAP2 inhibitor, or pharmaceutically acceptable salt thereof, and inavolisib (GDC-0077), or pharmaceutically acceptable salt thereof, can be administered in temporal proximity.

In some aspects, the MetAP2 inhibitor, or pharmaceutically acceptable salt thereof, and inavolisib (GDC-0077), or pharmaceutically acceptable salt thereof, can be administered in any order.

The present disclosure provides a method of preventing and/or mitigating treatment-induced hyperglycemia in a subject in need thereof, the method comprising administering to the subject at least one therapeutically effective amount of at least one MetAP2 inhibitor of the present disclosure, or a pharmaceutically acceptable salt thereof, at least one therapeutically effective amount of inavolisib (GDC-0077), or a pharmaceutically acceptable salt thereof.

The present disclosure provides a use of at least one MetAP2 inhibitor of the present disclosure, or a pharmaceutically acceptable salt thereof, in combination with inavolisib (GDC-0077), in the manufacture of a medicament for the prevention and/or the mitigation of treatment-induced hyperglycemia in a subject in need thereof.

The present disclosure provides a use of inavolisib (GDC-0077), or a pharmaceutically acceptable salt thereof, in combination with at least one MetAP2 inhibitor of the present disclosure, or a pharmaceutically acceptable salt thereof, in the manufacture of a medicament for the prevention and/or the mitigation of treatment-induced hyperglycemia in a subject in need thereof.

The present disclosure provides at least one MetAP2 inhibitor of the present disclosure, or a pharmaceutically acceptable salt thereof, for use in combination with inavolisib (GDC-0077), or a pharmaceutically acceptable salt thereof, in preventing and/or mitigating treatment-induced hyperglycemia in a subject in need thereof.

The present disclosure provides inavolisib (GDC-0077), or a pharmaceutically acceptable salt thereof, for use in combination with at least one MetAP2 inhibitor of the present disclosure, or a pharmaceutically acceptable salt thereof, in preventing and/or mitigating treatment-induced hyperglycemia in a subject in need thereof.

The present disclosure provides a combination of at least one MetAP2 inhibitor of the present disclosure, or a pharmaceutically acceptable salt thereof, and inavolisib (GDC-0077), or a pharmaceutically acceptable salt thereof, for use in preventing and/or mitigating treatment-induced hyperglycemia in a subject in need thereof.

### MetAP2 Inhibitors

Any of the MetAP2 inhibitors described herein can be used in the kits, pharmaceutical compositions, uses and methods described herein.

In some aspects, a MetAP2 inhibitor can be Compound 1, or a pharmaceutically acceptable salt, analog, derivative, salt or ester thereof, wherein Compound 1 is represented by:

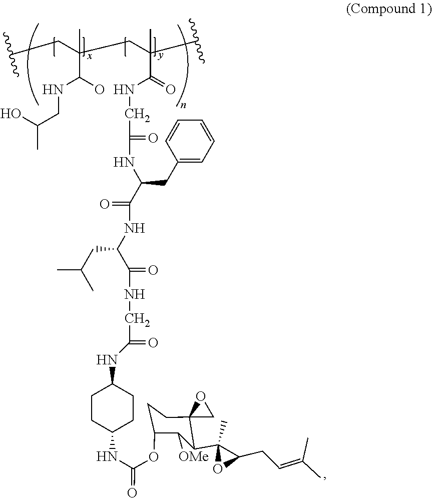

wherein x is in the range of 1 to about 450, y is in the range of 1 to about 30 and n is in the range of 1 to about 100. In some aspects, n is in the range of about 1 to about 90; about 1 to about 80; about 1 to about 70; about 1 to about 60; about 1 to about 55; or about 1 to about 50. In some aspects, the ratio of x to y can be in the range of about 30:1 to about 3:1.

In some aspects, a MetAP2 inhibitor can be Compound 2, or a pharmaceutically acceptable salt, analog, derivative, salt or ester thereof, wherein Compound 2 is represented by:

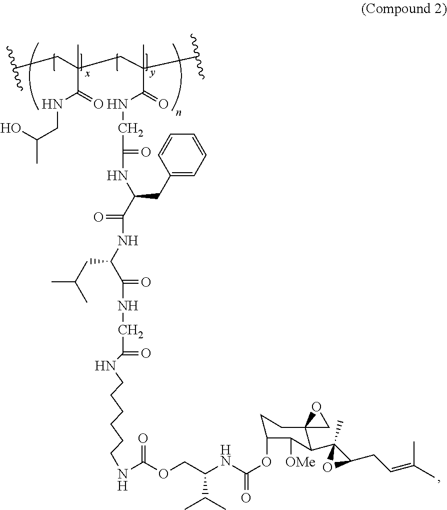

wherein x is in the range of 1 to about 450, y is in the range of 1 to about 30 and n is in the range of 1 to about 100. In some aspects, n is in the range of about 1 to about 90; about 1 to about 80; about 1 to about 70; about 1 to about 60; about 1 to about 55; or about 1 to about 50. In some aspects, the ratio of x to y can be in the range of about 30:1 to about 3:1.

In some aspects, a MetAP2 inhibitor can be Compound 3, or a pharmaceutically acceptable salt, analog, derivative, salt or ester thereof, wherein Compound 3 is represented by:

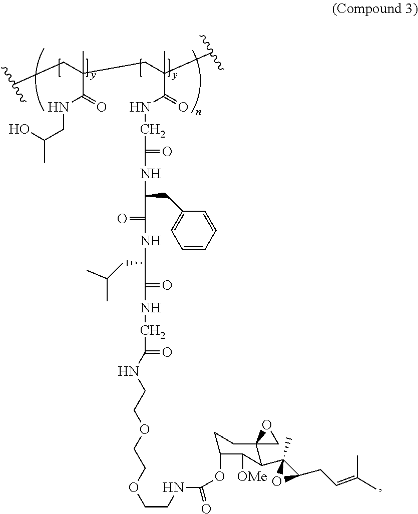

wherein x is in the range of 1 to about 450, y is in the range of 1 to about 30 and n is in the range of 1 to about 100. In some aspects, n is in the range of about 1 to about 90; about 1 to about 80; about 1 to about 70; about 1 to about 60; about 1 to about 55; or about 1 to about 50. In some aspects, the ratio of x to y can be in the range of about 30:1 to about 3:1.

In some aspects, a MetAP2 inhibitor can be Compound 4, or a pharmaceutically acceptable salt, analog, derivative, salt or ester thereof, wherein Compound 4 is represented by:

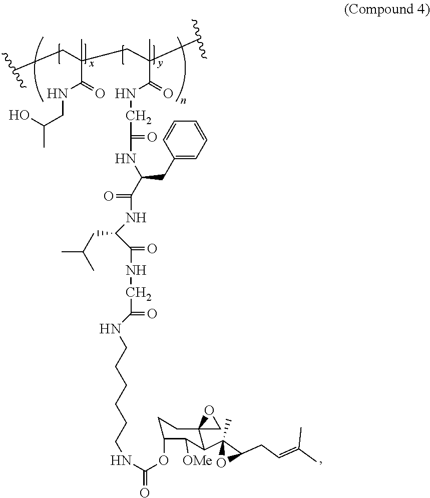

wherein x is in the range of 1 to about 450, y is in the range of 1 to about 30 and n is in the range of 1 to about 100. In some aspects, n is in the range of about 1 to about 90; about 1 to about 80; about 1 to about 70; about 1 to about 60; about 1 to about 55; or about 1 to about 50. In some aspects, the ratio of x to y can be in the range of about 30:1 to about 3:1.

In some aspects, the MetAP2 inhibitor can be:

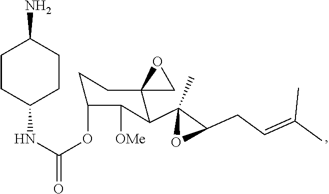

or a pharmaceutically acceptable salt, analog, derivative, salt or ester thereof.

In some aspects, the MetAP2 inhibitor can be:

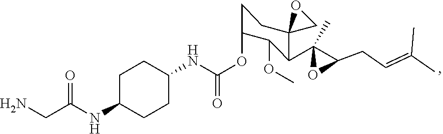

or a pharmaceutically acceptable salt, analog, derivative, salt or ester thereof.

In some aspects, the MetAP2 inhibitor can be:

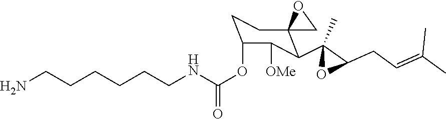

or a pharmaceutically acceptable salt, analog, derivative, salt or ester thereof.

In some aspects, the MetAP2 inhibitor can be:

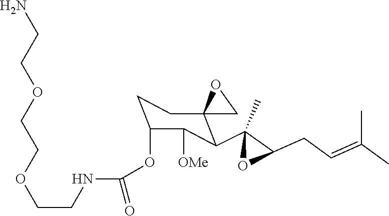

or a pharmaceutically acceptable salt, analog, derivative, salt or ester thereof.

In some aspects, the MetAP2 inhibitor can be:

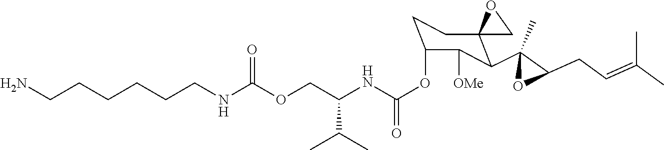

or a pharmaceutically acceptable salt, analog, derivative, salt or ester hereof.

In some aspects, the MetAP2 inhibitor can be selected from cis-(3aRS,9bRS)-7-(benzenesulfonylamino)-1,3a,4,9b-tetrahydro-2H-furo[2,3-c]chromene-6-carboxylic acid; cis-(3 aRS,9bRS)-7-[2-(3-diethylaminopropyl)-4-fluorobenzenesulfonyl-amino]-1,3a,4,9b-tetrahydro-2H-furo[2,3-c]chromene-6-carboxylic acid; cis-(3aRS,9bRS)-7-[2-(3-{pyrrolidin-1-yl}propyl)-4-fluorobenzenesulfonylamino]-1,3a,4,9b-tetrahydro-2H-furo[2,3-c]chromene-6-carboxylic acid; cis-(3aRS,9bRS)-7-[2-((Z)-3-diethylaminoprop-1-enyl)-4-fluorobenzenesulfonylamino]-1,3a,4,9b-tetrahydro-2H-furo[2,3-c]chromene-6-carboxylic acid; cis-(3aR,9bR)-7-[2-((Z)-3-diethylaminoprop-1-enyl)-4-fluoro-benzenesulfonylamino]-1,3a,4,9b-tetrahydro-2H-furo[2,3-c]chromene-6-carboxylic acid; cis-(3aS,9bS)-7-[2-((Z)-3-diethylaminoprop−1-enyl)-4-fluorobenzenesulfonylamino]-1,3a,4,9b-tetrahydro-2H-furo[2,3-c]chromene-6-carboxylic acid; 7-[2-((Z)-3-diethylaminoprop-1-enyl)-4-fluorobenzenesulfonylamino]-1,2-dihydrofuro[2,3-c]quinoline-6-carboxylic acid formate salt; 7-(benzenesulfonylamino))-1,2-dihydrofuro[2,3-c]quinoline-6-carboxylic acid formate salt; cis-(3aRS,9bR S)-7-[2-((Z)-3-diethylaminoprop-1-enyl)-4-fluorobenzenesulfonylamino]-1,2,3a,4,5,9b-hexahydrofuro[2,3-c]quinoline-6-carboxylic acid; (1 aRS,7bSR)-5-[2-((Z)-3-diethylaminoprop-1-enyl)-4-fluorobenzenesulfonylamino]-1,1a,2,7b-tetrahydrocyclopropa[c]chromene-4-carboxylic acid; (1aR,7bS)-5-[2-((Z)-3-diethylaminoprop−1-enyl)-4-fluorobenzenesulfonylamino]-1,1a,2,7b-tetrahydrocyclopropa[c]chromene-4-carboxylic acid; (1 aS,7bR)-5-[2-((Z)-3-diethylaminoprop-1-enyl)-4-fluorobenzenesulfonylamino]-1,1a,2,7b-tetrahydrocyclopropa[c]chromene-4-carboxylic acid; (1aRS,7bSR)-5-[2-((Z)-3-diethylaminoprop−1-enyl)-4-fluorobenzenesulfonylamino]-7b-methyl-1,1a,2,7b-tetrahydrocyclopropa[c]chromene-4-carboxylic acid; (1aRS,7bSR)-5-[2-((E)-3-diethylaminoprop-1-enyl)-4-fluorobenzenesulfonylamino]-7b-methyl-1,1a,2,7b-tetrahydrocyclopropa[c]chromene-4-carboxylic acid; cis-(3aRS,9bRS)-7-[2-(4-dimethylamino-butylamino)-benzenesulfonylamino]-1,3a,4,9b-tetrahydro-2H-furo[2,3-c]chromene-6-carboxylic acid; (1 aR,7bS)-5-[2-(3-diethylaminopropyl)-4-fluorobenzenesulfonyl-amino]-1,1a,2,7b-tetrahydrocyclopropa[c]chromene-4-carboxylic acid; (1aRS,7bSR)-5-[2-((Z)-3-diethylaminoprop-1-enyl)-4-fluorobenzene-sulfonylamino]-1,1-difluoro-1,1a,2,7b-tetrahydrocyclopropa[c]chromene-4-carboxylic acid; (1aR,7bS)-5-[2-((Z)-3-diethylaminoprop-1-enyl)-4-fluorobenzene-sulfonylamino]-1,1-difluoro-1,1a,2,7b-tetrahydrocyclopropa[c]chromene-4-carboxylic acid; (1 aS,7bR)-5-[2-((Z)-3-diethylaminoprop-1-enyl)-4-fluorobenzene-sulfonylamino]-1,1-difluoro-1,1a,2,7b-tetrahydrocyclopropa[c]chromene-4-carboxylic acid; (1aRS,7bSR)-5-[2((Z)-3-ethylaminoprop-1-enyl)-4-fluoro-benzenesulfonylamino]-1,1a,2,7b-tetrahydrocyclopropa-[c]chromene-4-carboxylic acid; (1aR,7bS)-5-[2((Z)-3-ethylaminoprop-1-enyl)-4-fluorobenzenesulfonylamino]-1,1a,2,7b-tetrahydrocyclopropa[c]chromene-4-carboxylic acid; (1aS,7bR)-5-[2((Z)-3-ethylaminoprop-1-enyl)-4-fluorobenzene-sulfonylamino]-1,1a,2,7b-tetrahydro-cyclopropa[c]chromene-4-carboxylic acid; (1aRS,7bSR)-5-{2[(Z)-3-(pyrrolidin-1-yl)prop-1-enyl]-4-fluorobenzenesulfonylamino}-1,1a,2,7b-tetrahydro-cyclopropa[c]chromene-4-carboxylic acid; (1aR,7bS)-5-{2[(Z)-3-(pyrrolidin-1-yl)prop-1-enyl]-4-fluorobenzenesulfonyl-amino}-1,1a,2,7b-tetrahydro-cyclopropa[c]chromene-4-carboxylic acid; (1 aS,7bR)-5-{2 [(Z)-3-(pyrrolidin-1-yl)prop-1-enyl]-4-fluorobenzenesulfonylamino}-1,1a,2,7b-tetrahydro-cyclopropa[c]chromene-4-carboxylic acid; (1aRS,7bSR)-5-[2-(3-dimethylaminopropylamino)-benzenesulfonylamino]-1,1a,2,7b-tetrahydrocyclopropa[c]chromene-4-carboxylic acid; (1 aR,7bS)-5-[2-(3-dimethylaminopropylamino)benzene-sulfonylamino]-1,1a,2,7b-tetrahydrocyclopropa[c]chromene-4-carboxylic acid; (1aS,7bR)-5-[2-(3-dimethylaminopropyl-amino)benzenesulfonylamino]-1,1a,2,7b-tetrahydrocyclopropa[c]chromene-4-carboxylic acid; (1aRS,7bSR)-5-[2-(4-dimethylaminobutylamino)benzenesulfonylamino]-1,1a,2,7b-tetrahydrocyclopropa[c]chromene-4-carboxylic acid; (1aR,7bS)-5-[2-(4-dimethylamino-butylamino)benzenesulfonylamino]-1,1a,2,7b-tetrahydrocyclopropa[c]chromene-4-carboxylic acid; (1aS,7bR)-5-[2-(4-dimethylaminobutylamino]-1,1a,2,7b-tetrahydrocyclopropa[c]chromene-4-carboxylic acid; (1 aRS,7bSR)-5-[2-(5-dimethylamino-pentylamino)benzene-sulfonylamino]-1,1a,2,7b-tetrahydrocyclopropa[c]chromene-4-carboxylic acid; (1aRS,7bSR)-5-{2[(Z)-3-(propan-2-yl)aminoprop-1-enyl]-4-fluorobenzenesulfonyl-amino}-1,1a,2,7b-tetrahydrocyclopropa[c]chromene-4-carboxylic acid; (1 aRS,7bSR)-5-{2[(Z)-3-((S)-3-hydroxypyrrolidin-1-yl)aminoprop-1-enyl]-4-fluorobenzenesulfonylamino}-1,1a,2,7b-tetrahydrocyclopropa[c]chromene-4-carboxylic acid; (1 aRS,7bSR)-5-{2[(Z)-3-((R)-3-hydroxypyrrolidin-1-yl)aminoprop-1-enyl]-4-fluorobenzene-sulfonylamino}-1,1a,2,7b-tetrahydro-cyclopropa[c]chromene-4-carboxylic acid; (1aRS,7bSR)-5-[2((Z)-4-diethylaminobut-1-enyl)-4-fluorobenzenesulfonyl-amino]-1,1a,2,7b-tetrahydrocyclopropa[c]chromene-4-carboxylic acid; (1aR,7bS)-5-[2((Z)-4-diethylaminobut-1-enyl)-4-fluorobenzenesulfonyl-amino]-1,1a,2,7b-tetrahydrocyclopropa[c]chromene-4-carboxylic acid; (1 aS,7bR)-5-[2((Z)-4-diethylaminobut−1-enyl)-4-fluorobenzenesulfonyl-amino]-1,1a,2,7b-tetrahydro-cyclopropa[c]chromene-4-carboxylic acid; (1 aRS,7bSR)-5-{2-[2-(4-ethylpiperazin-1-yl)-ethyl]-4-fluorobenzenesulfonylamino}-1,1a,2,7b-tetrahydrocyclopropa[c]chromene-4-carboxylic acid; (1aRS,7bSR)-5-{2[(Z)-3-(azetidin-1-yl)prop−1-enyl]-4-fluorobenzene-sulfonylamino}-1,1a,2,7b-tetrahydro-cyclopropa[c]chromene-4-carboxylic acid; (1aRS,7bSR)-5-{2[(Z)-3-(3-hydroxy-azetidin-1-yl)prop-1-enyl]-4-fluorobenzene-sulfonylamino}-1,1a,2,7b-tetrahydrocyclopropa-[c]chromene-4-carboxylic acid; (1aRS,7bSR)-5-(2[(Z)-3-(azetidin-1-yl)propyl]-4-fluorobenzenesulfonylamino]-1,1a,2,7b-tetrahydrocyclopropa[c]chromene-4-carboxylic acid; (1 aRS,7bSR)-5-[2((Z)-4-diethylaminobutyl)-4-fluorobenzenesulfonylamino]-1,1a,2,7b-tetrahydrocyclopropa[c]chromene-4-carboxylic acid; (1 aRS,7bSR)-5-{2-[N-(4-dimethylaminobutyl)-N-methylamino]-benzenesulfonyl-amino}-1,1a,2,7b-tetrahydrocyclopropa-[c]chromene-4-carboxylic acid; (1aRS,7bSR)-5-{2-[((S)-1-ethylpyrrolidin-3-ylcarbamoyl)-methyl]-4-fluoro-benzenesulfonyl-amino}-1,1a,2,7b-tetrahydrocyclopropa-[c]chromene-4-carboxylic acid; (1aRS,7bSR)-5-[2-(1-ethylazetidin-3-yl)-4-fluorobenzenesulfonylamino]-1,1a,2,7b-tetrahydro-cyclopropa[c]chromene-4-carboxylic acid; (1 aRS,7bSR)-5-{2-[((R)-1-ethylpyrrolidin-3-ylcarbamoyl)methyl]-4-fluorobenzenesulfonyl-amino}-1,1a,2,7b-tetrahydro-cyclopropa[c]chromene-4-carboxylic acid; (1 aRS,7bSR)-5-{2-[2-(pyrrolidin-1-yl)-ethyl]-4-fluorobenzenesulfonylamino}-1,1a,2,7b-tetrahydrocyclopropa[c]chromene-4-carboxylic acid; (1aRS,7bSR)-5-[2-((R)-1-ethylpyrrolidin-3-ylmethyl)-4-fluorobenzenesulfonyl-amino]-1,1a,2,7b-tetrahydro-cyclopropa[c]chromene-4-carboxylic acid; (1aS,7bR)-5-[2-((R)-1-ethylpyrrolidin-3-ylmethyl)-4-fluorobenzenesulfonyl-amino]-1,1a,2,7b-tetrahydro-cyclopropa[c]chromene-4-carboxylic acid; (1 aR,7bS)-5-[2-((R)-1-ethylpyrrolidin-3-ylmethyl)-4-fluorobenzenesulfonyl-amino]-1,1a,2,7b-tetrahydro-cyclopropa[c]chromene-4-carboxylic acid; (1aRS,7bSR)-5-{2-[((S)-1-ethylpyrrolidin-2-yl)carbonyl-aminomethyl]-4-fluorobenzene-sulfonylamino}-1,1a,2,7b-tetrahydrocyclopropa[c]chromene-4-carboxylic acid; (1 aRS,7bSR)-5-[2-(4-dimethylaminobutyrylamino)-4-fluorobenzenesulfonyl-amino]-1,1a,2,7b-tetrahydrocyclopropa-[c]chromene-4-carboxylic acid; (1aRS,7bSR)-5-[2-((S)-1-ethyl-pyrrolidin-3-ylmethyl)-4-fluorobenzenesulfonyl-amino]-1,1a,2,7b-tetrahydrocyclopropa[c]chromene-4-carboxylic acid; (1aRS,7bSR)-5-[2-(3-dimethylaminopropylcarbamoyl)benzene-sulfonylamino]-1,1a,2,7b-tetrahydrocyclopropa[c]chromene-4-carboxylic acid; (1 aRS,7bSR)-5-(2-{[N—((S)-1-ethyl-pyrrolidin-3-yl)-N-methylcarbamoyl]methyl}-4-fluoro-benzenesulfonylamino)-1,1a,2,7b-tetrahydrocyclopropa-[c]chromene-4-carboxylic acid; (1 aRS,7bSR)-5-(2-{[N—((R)-1-ethyl-pyrrolidin-3-yl)-N-methylcarbamoyl]methyl}-4-fluoro-benzenesulfonylamino)-1,1a,2,7b-tetrahydrocyclopropa-[c]chromene-4-carboxylic acid; (1 aRS,7bSR)-5-{2-[2-((S)-1-ethylpyrrolidin-2-yl)ethylamino]-benzenesulfonyl-amino}-1,1a,2,7b-tetrahydrocyclopropa-[c]chromene-4-carboxylic acid; (1aRS,7bSR)-5-[2-[2-((R)-1-ethylpyrrolidin-2-yl)ethylamino]-benzenesulfonyl-amino)-1,1a,2,7b-tetrahydrocyclopropa-[c]chromene-4-carboxylic acid; (1aRS,7bSR)-5-[2-(3-N,N,-diethylaminopropylamino)benzene-sulfonylamino]-1,1a,2,7b-tetrahydrocyclopropa[c]chromene-4-carboxylic acid; (1aRS,7bSR)-5-(2-([((R)-1-ethylpyrrolidine-2-yl)carbonyl-aminomethyl]-4-fluorobenzenesulfonylamino)-1,1a,2,7b-tetrahydrocyclopropa[c]chromene-4-carboxylic acid; (1aRS,7bSR)-5-{2-[(1-ethylazetidin-3-ylmethyl)amino]benzene-sulfonylamino}-1,1a,2,7b-tetrahydrocyclopropa[c]chromene-4-carboxylic acid; (1 aS,7bR)-5-[2-((Z)-3-diethylaminoprop-1-enyl)benzenesulfonylamino]-1,1a,2,7b-tetrahydrocyclopropa[c]chromene-4-carboxylic acid; (1 aR7bS)-5-[2-((Z)-3-diethylaminoprop-1-enyl)benzenesulfonylamino]-1,1a,2,7b-tetrahydrocyclopropa[c]chromene-4-carboxylic acid; (1 aRS,7bSR)-5-(2-{N—[((R)-1-ethylpyrrolidine-2-yl)carbonyl]-N-methyl-aminomethyl}-4-fluorobenzenesulfonylamino)-1,1a,2,7b-tetrahydrocyclopropa[c]chromene-4-carboxylic acid; (1 aRS,7bSR)-5-(2-{N—[((S)-1-ethylpyrrolidine-2-yl)carbonyl]-N-methylamino-methyl}-4-fluorobenzenesulfonylamino)-1,1a,2,7b-tetrahydrocyclopropa[c]chromene-4-carboxylic acid; (1 aRS,7bSR)-5-[2-(4-dimethylaminobutylamino)-4-fluorobenzenesulfonyl-amino]-1,1a,2,7b-tetrahydrocyclopropa[c]chromene-4-carboxylic acid; (1aRS,7bSR)-5-{2-[((R)-1-ethylpyrrolidin-3-ylmethyl)amino]-benzenesulfonylamino}-1,1a,2,7b-tetrahydrocyclopropa-[c]chromene-4-carboxylic acid; (1aRS,7bSR)-5-{2-[((S)-1-ethylpyrrolidin-3-ylmethyl)amino]-benzenesulfonylamino}-1,1a,2,7b-tetrahydrocyclopropa-[c]chromene-4-carboxylic acid; (1aRS,7bSR)-5-[2-(4-ethyl-2-oxopiperazin-1-ylmethyl)-4-fluorobenzene-sulfonylamino]-1,1a,2,7b-tetrahydrocyclopropa[c]chromene-4-carboxylic acid; (1aRS,7bSR)-5-[2-(1-ethylpiperidin-4-ylmethyl)-4-fluoro-benzenesulfonylamino]-1,1a,2,7b-tetrahydrocyclopropa-[c]chromene-4-carboxylic acid; (1 aRS,7bSR)-5-{2-[2-(1-ethylazetidin-3-yl)ethyl]-4-fluoro-benzenesulfonyl-amino}-1,1a,2,7b-tetrahydrocyclopropa[c]chromene-4-carboxylic acid; (1 aRS,7bSR)-5-(2-[((S)-1-azabicyclo[2.2.2]oct-3-yl)amino]benzenesulfonyl-amino)-1,1a,2,7b-tetrahydrocyclopropa[c]chromene-4-carboxylic acid; (1 aRS,7bSR)-5-{2-[((R)-1-azabicyclo-[2.2.2]oct-3-yl)amino]benzenesulfonyl-amino}-1,1a,2,7b-tetrahydrocyclopropa[c]chromene-4-carboxylic acid; (1 aRS,7bSR)-5-(2-{[((S)-1-ethylpyrrolidine-3-carbonyl)amino]methyl}-4-fluoro-benzenesulfonylamino)-1,1a,2,7b-tetrahydrocyclopropa[c]chromene-4-carboxylic acid; (1 aRS,7bSR)-5-{2-[2-((R)−1-ethylpyrrolidin-3-ylamino)ethyl]-4-fluoro-benzenesulfonylamino}-1,1a,2,7b-tetrahydrocyclopropa-[c]chromene-4-carboxylic acid; (1 aRS,7bSR)-5-{2-[((R)-1-ethylpyrrolidin-3-yl)amino]-benzenesulfonylamino}-1,1a,2,7b-tetrahydrocyclopropa-[c]chromene-4-carboxylic acid; (1aRS,7bSR)-5-{2-[((S)−1-ethylpyrrolidin-3-yl)amino]-benzenesulfonylamino}-1,1a,2,7b-tetrahydrocyclopropa-[c]chromene-4-carboxylic acid; (1 aRS,7bSR)-5-(2-{[((R)-1-ethylpyrrolidine-3-carbonyl)amino]-methyl)}-4-fluoro-benzenesulfonylamino)-1,1a,2,7b-tetrahydro-cyclopropa[c]chromene-4-carboxylic acid; (1aRS,7bSR)-5-[2-((Z)-3-diethylamino-2-methylprop-1-enyl)-4-fluorobenzene-sulfonylamino]-1,1a,2,7b-tetrahydrocyclopropa[c]chromene-4-carboxylic acid; (1 aRS,7bSR)-5-{2-[2-((R)-1-ethylpyrrolidin-3-yl)ethylamino]-benzenesulfonylamino}-1,1a,2,7b-tetrahydrocyclopropa-[c]chromene-4-carboxylic acid; (1aRS,7bSR)-5-{2-[2-((S)-1-ethylpyrrolidin-3-yl)ethylamino]-benzenesulfonyl-amino}-1,1a,2,7b-tetrahydrocyclopropa-[c]chromene-4-carboxylic acid; (1aR,7bS)-5-[2-((S)−1-ethylpyrrolidin-3-yloxymethyl)-4-fluoro-benzenesulfonylamino]-1,1a,2,7b-tetrahydrocyclopropa-[c]chromene-4-carboxylic acid; (1 aR,7bS)-5-[2-((R)-1-ethylpyrrolidin-3-yloxymethyl)-4-fluoro-benzenesulfonylamino]-1,1a,2,7b-tetrahydrocyclopropa-[c]chromene-4-carboxylic acid; (1aR,7bS)-5-[2-(1-ethylpiperidin-3-ylmethyl)-4-fluorobenzene-sulfonylamino]-1,1a,2,7b-tetrahydrocyclopropa[c]chromene-4-carboxylic acid; (1aR,7bS)-5-{2-[2-((R)-1-ethylpyrrolidin-2-yl)ethyl]-4-fluorobenzenesulfonyl-amino}-1,1a,2,7b-tetrahydrocyclopropa-[c]chromene-4-carboxylic acid; and pharmaceutically acceptable salts, stereoisomers, esters and prodrugs thereof.

In some aspects, a MetAP2 inhibitor can be selected from:

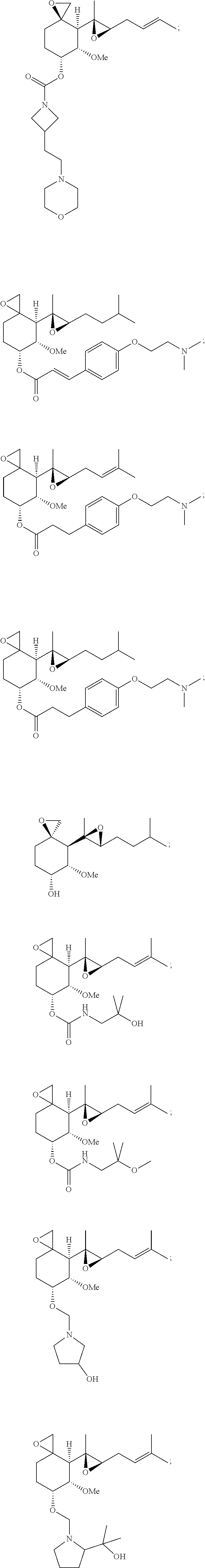

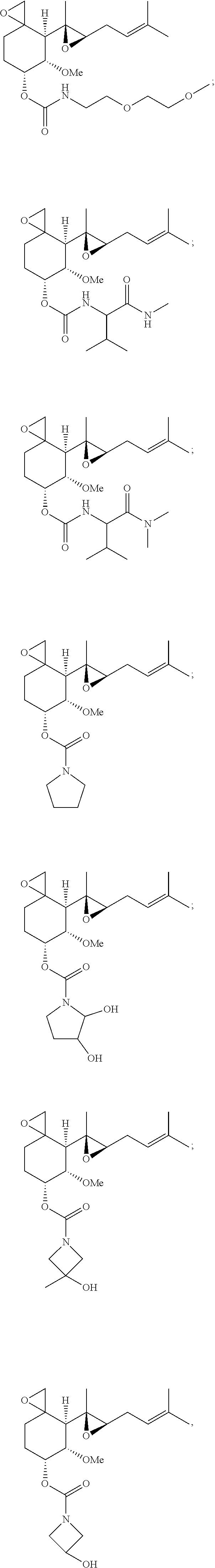

or a pharmaceutically acceptable salt, analog, derivative, salt or ester thereof.

In some aspects, a MetAP2 inhibitor can be:

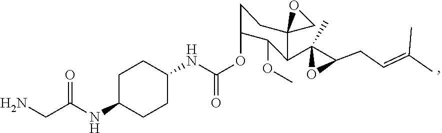

or a pharmaceutically acceptable salt, analog, derivative, salt or ester thereof.

In some aspects, a MetAP2 inhibitor can be administered by subcutaneous injection (SC). In some aspects, a MetAP2 inhibitor can be administered by subcutaneous injection to the mid-abdominal (per-umbilical area). In some aspects, a subcutaneous injection of a MetAP2 inhibitor can be administered over an about 30 to about 45 second timeframe at a constant injection rate. In some aspects, the maximum injection volume of a MetAP2 inhibitor is less than about 1.7 ml.

In some aspects, a MetAP2 inhibitor can be administered about every four days (Q4D).

In some aspects, a MetAP2 inhibitor can be administered about once every day (QD), about once every two days (Q2D), about once every three days (Q3D), about once every four days (Q4D), about once every 5 days (Q5D), about once every 6 days (Q6D), about once every 7 days (Q7D), about once every 8 days (Q8D), about once every 9 days (Q9D), about once every 10 days (Q10D), about once every 11 days (Q1D), about once every 12 days (Q12D), about once every 13 days (Q13D), about once every 14 days (Q14D), or about once every 15 days (Q1D). In some aspects, a MetAP2 inhibitor can be administered about once every 7 days (Q7D). In some aspects, a MetAP2 inhibitor can be administered about once every 14 days (Q14D).

In some aspects, a MetAP2 inhibitor can be administered in an amount of about 1 mg/m², or about 2 mg/m², or about 3 mg/m², or about 4 mg/m², or about 5 mg/m², or about 6 mg/m², or about 7 mg/m², or about 8 mg/m², or about 9 mg/m², or about 10 mg/m², or about 11 mg/m², or about 12 mg/m², or about 13 mg/m², or about 14 mg/m², or about 15 mg/m², or about 16 mg/m², or about 17 mg/m², or about 18 mg/m², or about 19 mg/m², or about 20 mg/m², or about 21 mg/m², or about 22 mg/m², or about 23 mg/m², or about 24 mg/m², or about 25 mg/m², or about 26 mg/m², or about 27 mg/m², or about 28 mg/m², or about 29 mg/m², or about 30 mg/m², or about 31 mg/m², or about 32 mg/m², or about 33 mg/m², or about 34 mg/m², or about 35 mg/m², or about 36 mg/m², or about 37 mg/m², or about 38 mg/m², or about 39 mg/m², or about 40 mg/m², or about 41 mg/m², or about 42 mg/m², or about 43 mg/m², or about 44 mg/m², or about 45 mg/m², or about 46 mg/m², or about 47 mg/m², or about 48 mg/m², or about 49 mg/m², or about 50 mg/m², or about 51 mg/m², or about 52 mg/m², or about 53 mg/m², or about 54 mg/m², or about 55 mg/m², or about 56 mg/m², or about 57 mg/m², or about 58 mg/m², or about 59 mg/m², or about 60 mg/m², or about mg/m², or about 61 mg/m², or about 62 mg/m², or about 63 mg/m², or about 64 mg/m², or about 65 mg/m², or about 66 mg/m², or about 67 mg/m², or about 68 mg/m², or about 69 mg/m², or about 70 mg/m², or about 81 mg/m², or about 82 mg/m², or about 83 mg/m², or about 84 mg/m², or about 85 mg/m², or about 86 mg/m², or about 87 mg/m², or about 88 mg/m², or about 89 mg/m², or about 90 mg/m², or about 91 mg/m², or about 92 mg/m², or about 93 mg/m², or about 94 mg/m², or about 95 mg/m², or about 96 mg/m², or about 97 mg/m², or about 98 mg/m², or about 99 mg/m², or about 100 mg/m².

In some aspects, a MetAP2 inhibitor can be administered in an amount of about 49 mg/m². In some aspects, a MetAP2 inhibitor can be administered in an amount of about 36 mg/m². In some aspects, a MetAP2 inhibitor can be administered in an amount of about 65 mg/m². In some aspects, a MetAP2 inhibitor can be administered in an amount of about 27 mg/m².

In some aspects, a MetAP2 inhibitor can be administered in an amount of about 49 mg/m². In some aspects, a MetAP2 inhibitor can be administered in an amount of about 39 mg/m² to about 59 mg/m². In some aspects, a MetAP2 inhibitor can be administered in an amount of about 44 mg/m² to about 54 mg/m².

In some aspects, a MetAP2 inhibitor can be administered in an amount of about 36 mg/m². In some aspects, a MetAP2 inhibitor can be administered in an amount of about 26 mg/m² to about 49 mg/m². In some aspects, a MetAP2 inhibitor can be administered in an amount of about 31 mg/m² to about 65 mg/m².

In some aspects, a MetAP2 inhibitor can be administered in an amount of about 65 mg/m². In some aspects, a MetAP2 inhibitor can be administered in an amount of about 55 mg/m² to about 75 mg/m². In some aspects, a MetAP2 inhibitor can be administered in an amount of about 60 mg/m² to about 70 mg/m².

In some aspects, a therapeutically effective amount of a MetAP2 inhibitor can be about 1 mg/m², or about 2 mg/m², or about 3 mg/m², or about 4 mg/m², or about 5 mg/m², or about 6 mg/m², or about 7 mg/m², or about 8 mg/m², or about 9 mg/m², or about 10 mg/m², or about 11 mg/m², or about 12 mg/m², or about 13 mg/m², or about 14 mg/m², or about 15 mg/m², or about 16 mg/m², or about 17 mg/m², or about 18 mg/m², or about 19 mg/m², or about 20 mg/m², or about 21 mg/m², or about 22 mg/m², or about 23 mg/m², or about 24 mg/m², or about 25 mg/m², or about 26 mg/m², or about 27 mg/m², or about 28 mg/m², or about 29 mg/m², or about 30 mg/m², or about 31 mg/m², or about 32 mg/m², or about 33 mg/m², or about 34 mg/m², or about 35 mg/m², or about 36 mg/m², or about 37 mg/m², or about 38 mg/m², or about 39 mg/m², or about 40 mg/m², or about 41 mg/m², or about 42 mg/m², or about 43 mg/m², or about 44 mg/m², or about 45 mg/m², or about 46 mg/m², or about 47 mg/m², or about 48 mg/m², or about 49 mg/m², or about 50 mg/m², or about 51 mg/m², or about 52 mg/m², or about 53 mg/m², or about 54 mg/m², or about 55 mg/m², or about 56 mg/m², or about 57 mg/m², or about 58 mg/m², or about 59 mg/m², or about 60 mg/m², or about mg/m², or about 61 mg/m², or about 62 mg/m², or about 63 mg/m², or about 64 mg/m², or about 65 mg/m², or about 66 mg/m², or about 67 mg/m², or about 68 mg/m², or about 69 mg/m², or about 70 mg/m², or about 81 mg/m², or about 82 mg/m², or about 83 mg/m², or about 84 mg/m², or about 85 mg/m², or about 86 mg/m², or about 87 mg/m², or about 88 mg/m², or about 89 mg/m², or about 90 mg/m², or about 91 mg/m², or about 92 mg/m², or about 93 mg/m², or about 94 mg/m², or about 95 mg/m², or about 96 mg/m², or about 97 mg/m², or about 98 mg/m², or about 99 mg/m², or about 100 mg/m².

In some aspects, a therapeutically effective amount of a MetAP2 inhibitor can be about 49 mg/m². In some aspects, a therapeutically effective amount of a MetAP2 inhibitor can be about 39 mg/m² to about 59 mg/m². In some aspects, a therapeutically effective amount of a MetAP2 inhibitor can be about 44 mg/m² to about 54 mg/m².

In some aspects, a therapeutically effective amount of a MetAP2 inhibitor can be about 36 mg/m². In some aspects, a therapeutically effective amount of a MetAP2 inhibitor can be about 26 mg/m² to about 49 mg/m². In some aspects, a therapeutically effective amount of a MetAP2 inhibitor can be about 31 mg/m² to about 49 mg/m².

In some aspects, a therapeutically effective amount of a MetAP2 inhibitor can be about 65 mg/m². In some aspects, a therapeutically effective amount of a MetAP2 inhibitor can be about 55 mg/m² to about 75 mg/m². In some aspects, a therapeutically effective amount of a MetAP2 inhibitor can be about 60 mg/m² to about 70 mg/m².

In some aspects, a MetAP2 inhibitor can be administered in an amount of about 10 mg, or about 20 mg, or about 30 mg, or about 40 mg, or about 50 mg, or about 60 mg, or about 70 mg, or about 80 mg, or about 90 mg, or about 100 mg or about 110 mg, or about 120 mg, or about 130 mg, or about 140 mg, or about 150 mg, or about 160 mg, or about 170 mg, or about 180 mg, or about 190 mg, or about 200 mg. In some aspects a MetAP2 inhibitor can be administered in an amount of about 80 mg. In some aspects, a MetAP2 inhibitor can be administered in an amount of about of about 70 mg to about 90 mg. In some aspects, a MetAP2 inhibitor can be administered in an amount of about 75 mg to about 85 mg.

In some aspects, a therapeutically effective amount of a MetAP2 inhibitor can be about 10 mg, or about 20 mg, or about 30 mg, or about 40 mg, or about 50 mg, or about 60 mg, or about 70 mg, or about 80 mg, or about 90 mg, or about 100 mg or about 110 mg, or about 120 mg, or about 130 mg, or about 140 mg, or about 150 mg, or about 160 mg, or about 170 mg, or about 180 mg, or about 190 mg, or about 200 mg. In some aspects a therapeutically effective amount of a MetAP2 inhibitor can be about 80 mg. In some aspects, a therapeutically effective amount of a MetAP2 inhibitor can be about of about 70 mg to about 90 mg. In some aspects, a therapeutically effective amount of a MetAP2 inhibitor can be about 75 mg to about 85 mg.

### Selective Estrogen Receptor Degraders (SERDs)

Any of the selective estrogen receptor degraders (SERDs) described herein can be used in the kits, pharmaceutical compositions, uses and methods described herein.

As would be appreciated by the skilled artisan SERDs are compounds that bind to the estrogen receptor (ER) and causes the ER to be degraded and thus downregulated.

In some aspects, a SERD can be fulvestrant:

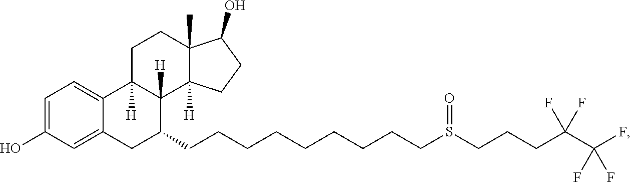

or a pharmaceutically acceptable salt, analog, derivative, salt or ester thereof. As would be appreciated by the skilled artisan, it is understood that fulvestrant may be identified by any one of the following names: 7α-[9-[(4,4,5,5,5-pentafluoropentyl)sulfinyl]nonyl]estradiol, 7α-[9-[(4,4,5,5,5-Pentafluoropentyl)-sulfinyl]nonyl]estra-1,3,5(10)-triene-3,7β-diol, ICI-182780, ZD-182780, and ZD-9238. As would be appreciated by the skilled artisan, fulvestrant may be identified as CAS No. 129453-61-8.

In some aspects, a fulvestrant can be administered intramuscularly.

In some aspects, fulvestrant can be administered in an amount of about 75 mg, or about 100 mg, or about 125 mg, or about 150 mg, or about 175 mg, or about 200 mg, or about 225 mg, or about 250 mg, or about 275 mg, or about 300 mg, or about 325 mg, or about 350 mg, or about 375 mg, or about 400 mg, or about 425 mg, or about 450 mg, or about 500 mg, or about 525 mg, or about 550 mg, or about 575 mg, or about 600 mg, or about 625 mg, or about 650 mg, or about 675 mg, or about 700 mg.

In some aspects, fulvestrant can be administered in an amount of about 500 mg. In aspects wherein fulvestrant is administered in an amount of about 500 mg, the amount can be administered by two intramuscular injections of about 250 mg of fulvestrant.

In some aspects, fulvestrant can be administered in an amount of about 250 mg. In aspects wherein fulvestrant is administered in an amount of about 250 mg, the amount can be administered in a single intramuscular injection of about 250 mg of fulvestrant.

In some aspects, a therapeutically effective amount of fulvestrant can be any of the fulvestrant amounts described herein.

In some aspects, an amount of about 250 mg of fulvestrant can be administered as an intramuscular injection, wherein the volume of the injection is about 5 ml, and wherein the injection is performed over a time period of about 1 to about 2 minutes.

In some aspects, fulvestrant can be administered as a pharmaceutical composition, wherein the pharmaceutical composition comprises at least one of alcohol, benzyl alcohol, benzyl benzoate as co-solvents. In some aspects, fulvestrant can be administered as a pharmaceutical composition, wherein the pharmaceutical composition comprises castor oil as a co-solvent and release rate modifier.

In some aspects, fulvestrant can be administered about once every two weeks. In some aspects, fulvestrant can be administered about once every two weeks over the course of about a month, and thereafter be administered one a month.

A non-limiting dosing schedule of fulvestrant can comprise administering an amount of fulvestrant (e.g. about 500 mg or about 250 mg) on Day 1, Day 15, Day 29 and then once monthly thereafter.

In some aspects, a SERD can be selected from brilanestrant, elacestrant, Giredestrant, Amcenestrant (SAR439859), AZD9833, Rintodestrant, LSZ102, LY3484356, Elacestrant, ZN-c5, D-0502, SHR9549 and any other SERD known in the art.

### PI3K Inhibitors

Any of the PI3K inhibitors described herein can be used in the kits, pharmaceutical compositions, uses and methods described herein.

As would be appreciated by the skilled artisan, PI3K inhibitors are compounds that bind inhibit one or more of phosphoinositide 3-kinase enzymes. In some aspects, a PI3K inhibitor can target 1 or more (e.g. 2, 3, 4 or more) isoforms of PI3K enzymes. In some aspects, a PI3K inhibitor can target the alpha-specific subunit. In some aspects, a PI3K inhibitor can be part of a pan-PI3K targeting treatment. In some aspects, a PI3K inhibitor can be a PI3Kα inhibitor.

In some aspects, a PI3K inhibitor can be alpelisib:

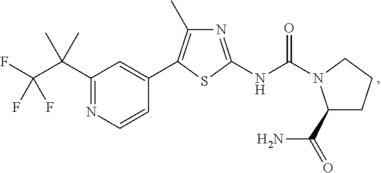 [SMILES: Cc1nc(NC(=O)N2CCC[C@H]2C(N)=O)sc1-c1ccnc(C(C)(C)C(F)(F)F)c1]

or a pharmaceutically acceptable salt, analog, derivative, salt or ester thereof. As would be appreciated by the skilled artisan, it is understood that alpelisib may be identified by any one of the following names: BYL-719 and (2S)-1-N-[4-Methyl-5-[2-(1,1,1-trifluoro-2-methylpropan-2-yl)pyridin-4-yl]-1,3-thiazol-2-yl]pyrrolidine-1,2-dicarboxamide. As would be appreciated by the skilled artisan, alpelisib may be identified as CAS No. 1217486-61-7.

In some aspects, alpelisib can be administered orally.

In some aspects, alpelisib can be administered in an amount of about 10 mg, or about 25 mg, or about 50 mg, or about 75 mg, or about 100 mg, or about 125 mg, or about 150 mg, or about 175 mg, or about 200 mg, or about 225 mg, or about 250 mg, or about 275 mg, or about 300 mg, or about 325 mg, or about 350 mg, or about 375 mg, or about 400 mg, or about 425 mg, or about 450 mg, or about 500 mg.

In some aspects, alpelisib can be administered in an amount of about 300 mg. In aspects wherein alpelisib is administered in an amount of about 300 mg, the amount can be administered administering to a subject two tablets each comprising about 150 mg of alpelisib.

In some aspects, alpelisib can be administered in an amount of about 250 mg. In aspects wherein alpelisib is administered in an amount of about 250 mg, the amount can be administered administering to a subject two tablets, one of the tablets comprising about 200 mg of alpelisib and the other tablet comprising about 50 mg of alpelisib.

In some aspects, alpelisib can be administered in an amount of about 200 mg. In aspects wherein alpelisib is administered in an amount of about 200 mg, the amount can be administered administering to a subject a tablet comprising about 200 mg of alpelisib.

In some aspects, alpelisib can be administered about once daily. Accordingly, alpelisib can be administered in an amount of about 300 mg/day, or about 250 mg/day, or about 200 mg/day.

In some aspects, a therapeutically effective amount of alpelisib can be any of the alpelisib amounts described herein.

In some aspects, alpelisib can be administered as a pharmaceutical composition, wherein the pharmaceutical composition comprises at least one of hypromellose, magnesium stearate, mannitol, microcrystalline cellulose and sodium starch glycolate. In some aspects, alpelisib can be administered as a pharmaceutical composition, wherein the pharmaceutical composition comprises at least one of hypromellose, magnesium stearate, mannitol, microcrystalline cellulose, sodium starch glycolate, iron oxide black, iron oxide red, macrogol/polyethylene glycol (PEG) 4000, talc, and titanium dioxide.

In some aspects, alpelisib can be administered as a tablet, wherein the tablet comprises alpelisib in combination with iron oxide black, iron oxide red, macrogol/polyethylene glycol (PEG) 4000, talc, and titanium dioxide, and wherein the tablet has a film-coating comprising hypromellose, iron oxide black, iron oxide red, macrogol/polyethylene glycol (PEG) 4000, talc, and titanium dioxide. In some aspects, a tablet can comprise about 50 mg of alpelisib. In some aspects, a tablet can comprise about 150 mg of alpelisib. In some aspects, a tablet can comprise about 200 mg of alpelisib.

In some aspects, alpelisib can be administered in combination with at least one antihistamine. As would be appreciated by the skilled artisan, the antihistamines can be administered in combination with alpelisib as a means of minimizing skin irritations or rashes resulting from exposure to alpelisib. Accordingly, any of the methods of the present disclosure can further comprise administering at least one antihistamine to a subject.

In some aspects, alpelisib can be administered in combination with metformin, insulin, an SGLT2 inhibitor, insulin sensitizers (e.g. thiazolidinediones, dipeptidyl peptidase-4 inhibitors) or any combination thereof, as a means of treating alpelisib-induced hyperglycemia. Accordingly, any of the methods of the present disclosure can further comprise administering metformin, insulin, an SGLT2 inhibitor, insulin sensitizers (e.g. thiazolidinediones, dipeptidyl peptidase-4 inhibitors) or any combination thereof.

In some aspects, a PI3K inhibitor can be selected from pictilisib, Ly294002, PI-103, ZSTK-474, alpelisib, AS-605240, PIK-75, A66, voxtalisib, PIK90, PF-04691502, AZD6482, apitolisib, GSK1059615, BGT226, fimepinostat, CH5132799, PKI-402, TG100713, VS-5584, KU-0060648, GNE-477, leniolisib, SF2523, AZD8835, AZD8186, PF-4989216, HS-173, copanlisib, idelalisib, buparlisib, inavolisib, paxalisib, rigosertib, bimiralisib, CUDC-908, dactolisib, GDC-0326, gedatolisib, omipalisib, pictilisib, HHCYH-33, men-1611, pilaralisib, serabelisib (TAK-117), GNE-477, teselisib, TL-117, HEC-68498, RLY-2608, or any other PI3K inhibitor known in the art.

As would be appreciated by the skilled artisan, inavolisib (GDC-0077) structure:

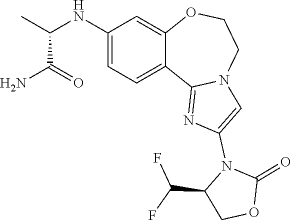 [SMILES: C[C@H](Nc1ccc2c(c1)OCCn1cc(N3C(=O)OC[C@H]3C(F)F)nc1-2)C(N)=O]

(see Hanan et al. Discovery of GDC-0077 [Inavolisib], a Highly Selective Inhibitor and Degrader of Mutant PI3Kα, Journal of Medicinal Chemistry, 2022, 65, 24, 16589-16621, which is incorporated herein by reference in its entirety for all purposes).

In some aspects, inavolisib, or a pharmaceutically acceptable salt thereof can be administered intravenously.

In some aspects, inavolisib can be administered orally.

In some aspects, inavolisib can be administered in an amount of about 0.5 mg, or about 1 mg, or about 1.5 mg, or about 2 mg, or about 2.5 mg, or about 3 mg, or about 3.5 mg, or about 4 mg, or about 4.5 mg, or about 5 mg, or about 5.5 mg, or about 6 mg, or about 6.5 mg, or about 7 mg, or about 7.5 mg, or about 8 mg, or about 8.5 mg, or about 9 mg, or about 9.5 mg, or about 10 mg, or about 10.5 mg, or about 11 mg, or about 11.5 mg, or about 12 mg, or about 12.5 mg, or about 13 mg, or about 13.5 mg, or about 14 mg, or about 14.5 mg, or about 15 mg, or about 15.5 mg, or about 16 mg, or about 16.5 mg, or about 17 mg, or about 17.5 mg, or about 18 mg, or about 18.5 mg, or about 19 mg, or about 19.5 mg, or about 20 mg.

In some aspects, a therapeutically effective amount of inavolisib can be any of the inavolisib amounts described herein.

In some aspects, inavolisib can be administered about once per week, about twice per week, about three times per week, about four times per week, about five times per week, about six times per week. In some aspects, inavolisib can be administered daily. In some aspects, inavolisib can be administered daily on Days 1-28 of a 28-day cycle.

### Microtubule-Targeting Compounds

Any of the microtubule-targeting compounds described herein can be used in the kits, pharmaceutical compositions, uses and methods described herein.

As would be appreciated by the skilled artisan, microtubule-targeting compounds (also referred to as microtubule-targeting agents in the art) are compounds that directly or indirectly modulate microtubule dynamics, thereby modulating various cellular functions, including, but not limited to cell cycle progression, mitosis, metabolism, cellular signaling, intracellular transport, etc.

In some aspects, a microtubule-targeting compound can be eribulin:

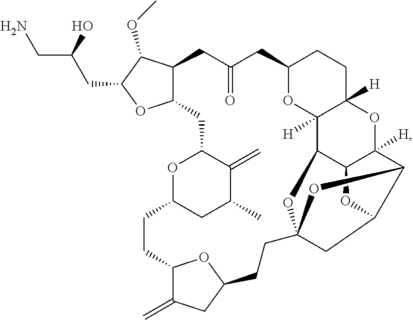

or a pharmaceutically acceptable salt, analog, derivative, salt or ester thereof. As would be appreciated by the skilled artisan, it is understood that eribulin may be identified by any one of the following names: E7389, ER-086526, NSC-707389, and 2-(3-Amino-2-hydroxypropyl)hexacosahydro-3-methoxy-26-methyl-20,27-bis(methylene)11,15-18,21-24,28-triepoxy-7,9-ethano-12,15-methano-9H,15H-furo(3,2-i)furo(2′,3′-5,6) pyrano(4,3-b)(1,4)dioxacyclopentacosin-5-(4H)-one. As would be appreciated by the skilled artisan, eribulin may be identified as CAS No. 253128-41-5.

In some aspects, the microtubule-targeting compound can be the mesylate salt of eribulin (eribulin mesylate).

In some aspects, eribulin, or a pharmaceutically acceptable salt, analog, derivative, salt or ester thereof can be administered intravenously.

In some aspects, eribulin mesylate can be administered intravenously.

In some aspects, eribulin can be administered in an amount of about 0.1 mg/m², or about 0.2 mg/m², or about 0.3 mg/m², or about 0.4 mg/m², or about 0.5 mg/m², or about 0.6 mg/m², or about 0.7 mg/m², or about 0.8 mg/m², or about 0.9 mg/m², or about 1.0 mg/m², or about 1.1 mg/m², or about 1.2 mg/m², or about 1.3 mg/m², or about 1.4 mg/m², or about 1.5 mg/m², or about 1.6 mg/m², or about 1.7 mg/m², or about 1.8 mg/m², or about 1.9 mg/m², or about 2.0 mg/m², or about 2.1 mg/m², or about 2.2 mg/m², or about 2.3 mg/m², or about 2.4 mg/m², or about 2.5 mg/m², or about 2.6 mg/m², or about 2.7 mg/m², or about 2.8 mg/m², or about 2.9 mg/m², or about 3.0 mg/m².

In some aspects, eribulin mesylate can be administered in an amount of about 0.1 mg/m², or about 0.2 mg/m², or about 0.3 mg/m², or about 0.4 mg/m², or about 0.5 mg/m², or about 0.6 mg/m², or about 0.7 mg/m², or about 0.8 mg/m², or about 0.9 mg/m², or about 1.0 mg/m², or about 1.1 mg/m², or about 1.2 mg/m², or about 1.3 mg/m², or about 1.4 mg/m², or about 1.5 mg/m², or about 1.6 mg/m², or about 1.7 mg/m², or about 1.8 mg/m², or about 1.9 mg/m², or about 2.0 mg/m², or about 2.1 mg/m², or about 2.2 mg/m², or about 2.3 mg/m², or about 2.4 mg/m², or about 2.5 mg/m², or about 2.6 mg/m², or about 2.7 mg/m², or about 2.8 mg/m², or about 2.9 mg/m², or about 3.0 mg/m².

In some aspects, eribulin can be administered in an amount of about 1.4 mg/m². In some aspects, eribulin can be administered in an amount of about 1.1 mg/m². In some aspects, eribulin can be administered in an amount of about 0.7 mg/m².

In some aspects, eribulin mesylate can be administered in an amount of about 1.4 mg/m². In some aspects, eribulin mesylate can be administered in an amount of about 1.1 mg/m². In some aspects, eribulin mesylate can be administered in an amount of about 0.7 mg/m².

In some aspects, wherein an amount of eribulin or eribulin mesylate is administered intravenously to a subject, the amount of eribulin or eribulin mesylate can be administered over a time period of about 2 minutes to about 5 minutes.

In some aspects, a therapeutically effective amount of eribulin can be any of the eribulin amounts described herein.

In some aspects, eribulin or eribulin mesylate can be administered about once a week. In some aspects, eribulin or eribulin mesylate can be administered on Days 1 and 8 of a 21-day cycle.

In some aspects, eribulin or eribulin mesylate can be administered as a pharmaceutical composition, wherein the pharmaceutical composition further comprises ethanol and water in a ratio of about 5:95. In some aspects, the concentration of eribulin or eribulin mesylate can be about 0.5 mg/ml in the pharmaceutical composition.

In some aspects, a microtubule-targeting compound can be selected from paclitaxel, docetaxel, vincristin, vinbiastin, nocodazole, epothilones, navelbine and any other microtubule-targeting compound known in the art.

### Treated Subjects and Cancers

In some aspects, the subject in need thereof is an animal. In some aspects, the animal can be a mammal. In some aspects, the subject in need thereof is a human.

In some aspects, the subject in need thereof is a human of 18 years or older. In some aspects, the subject in need thereof is a human younger than 18 years.

In some aspects, the subject in need thereof is a post-menopausal woman.

In some aspects, the subject in need thereof has a cancer. In some aspects, the cancer is characterized by at least one tumor present in the subject.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, leukemia and germ cell tumors. More particular examples of such cancers include adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer, cervical cancer, Acute lymphoblastic leukemia (ALL), Acute myeloid leukemia (AML), Adrenal gland tumors, Anal cancer, Bile duct cancer, Bladder cancer, Bone cancer, Bowel cancer, Brain tumors, Breast cancer, Cancer of unknown primary (CUP), Cancer spread to bone, Cancer spread to brain, Cancer spread to liver, Cancer spread to lung, Carcinoid, Cervical cancer, Children's cancers, Chronic lymphocytic leukemia (CLL), Chrome myeloid leukemia (CML), Colorectal cancer, Ear cancer, Endometrial cancer, Eye cancer, Follicular dendritic cell sarcoma, Gallbladder cancer, Gastric cancer, Gastro esophageal junction cancers, Germ cell tumors, Gestational trophoblastic disease (GIT)), Hairy cell leukemia, Head and neck cancer, Hodgkin lymphoma, Kaposi's sarcoma, Kidney cancer, Laryngeal cancer, Leukemia, Gastric linitis plastica, Liver cancer, Lung cancer, Lymphoma, Malignant schwannoma, Mediastinal germ cell tumors, Melanoma skin cancer, Men's cancer, Merkel cell skin cancer, Mesothelioma, Molar pregnancy, Mouth and oropharyngeal cancer, Myeloma. Nasal and paranasal sinus cancer, Nasopharyngeal cancer, Neuroblastoma, Neuroendocrine tumors, Non-Hodgkin lymphoma (NHL), Esophageal cancer, Ovarian cancer, Pancreatic cancer, Penile cancer, Persistent trophoblastic disease and choriocarcinoma, Pheochromocytoma, Prostate cancer, Pseudomyxoma peritonei, Rectal cancer. Retinoblastoma, Salivary gland cancer, Secondary cancer, Signet cell cancer, Skin cancer, Small bowel cancer, Soft tissue sarcoma, Stomach cancer, T cell childhood non Hodgkin lymphoma (NHL), Testicular cancer, Thymus gland cancer, Thyroid cancer, Tongue cancer, Tonsil cancer, Tumors of the adrenal gland, Uterine cancer. Vaginal cancer, Vulval cancer, Wilms' tumor, Womb cancer and Gynaecological cancer. Examples of cancer also include, but are not limited to, Hematologic malignancies, Lymphoma, Cutaneous T-cell lymphoma, Peripheral T-cell lymphoma, Hodgkin's lymphoma, Non-Hodgkin's lymphoma, Multiple myeloma, Chrome lymphocytic leukemia, chronic myeloid leukemia, acute myeloid leukemia, Myelodysplastic syndromes, Myelofibrosis, Biliary tract cancer, Hepatocellular cancer, Colorectal cancer, Breast cancer, Lung cancer, Non-small cell lung cancer, Ovarian cancer, Thyroid Carcinoma, Renal Cell Carcinoma, Pancreatic cancer, Bladder cancer, skin cancer, malignant melanoma, merkel cell carcinoma, Uveal Melanoma or Glioblastoma multiforme.

In some aspects, the cancer is a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a kidney cancer, a gastric cancer, a thyroid cancer, a pancreatic cancer, an esophageal cancer, a prostate cancer, a cervical cancer, a uterine cancer, a stomach cancer, a soft tissue cancer, a laryngeal cancer, a small intestine cancer, a testicular cancer, an anal cancer, a vulvar cancer, a joint cancer, an oral cancer, a pharynx cancer or a colorectal cancer.

In some aspects, the cancer is breast cancer.

In some aspects, the breast cancer is advanced breast cancer.

In some aspects, the breast cancer is metastatic breast cancer. As used herein, metastatic breast cancer is stage III or IV breast cancer that has spread to another part of the body, including, but not limited to, the liver, brain, bones, etc.

In some aspects, the breast cancer is human epidermal growth factor 2 (HER2)-negative breast cancer.

In some aspects, the breast cancer is a hormone receptor (HR)-positive breast cancer

In some aspects, the breast cancer is HR-positive/HER2-negative (HR+HER2−) breast cancer.

In some aspects, the breast cancer is triple-negative breast cancer. In some aspects, the breast cancer is triple-negative metastatic breast cancer.

In some aspects, a subject can be identified as having triple-negative breast cancer when samples from the subject exhibit estrogen and progesterone receptor staining is less than about 10% and HER2 immunohistochemistry (IHC) scores of 0 to 1+, as would be appreciated by the skilled artisan.

In some aspects, the subject in need thereof is a postmenopausal woman having HR+HER2− breast cancer.

In some aspects, the breast cancer can be a Luminal A breast cancer. In some aspects, the breast cancer can be a Luminal B breast cancer. In some aspects, the breast cancer can be a triple-negative or basal-like breast cancer. In some aspects the breast cancer can be a HER2-enriched breast cancer.

In some aspects, a subject in need thereof has breast cancer and at least one PIK3CA mutation.

In some aspects, a subject in need thereof is a postmenopausal woman having HR+HER2− breast cancer, wherein the subject in need thereof has at least one PIK3CA mutation.

In some aspects, a subject in need thereof has relapsed breast cancer, wherein the subject has been previously treated with neoadjuvant and/or adjuvant endocrine therapy. In some aspects, a subject in need thereof is a postmenopausal woman having relapsed HR+HER2− breast cancer, wherein the subject in need thereof has at least one PIK3CA mutation, wherein the subject has been previously treated with neoadjuvant and/or adjuvant endocrine therapy. In some aspects, the relapsed breast cancer is characterized by progressive disease more than about 12 months from the completion of the neoadjuvant and/or adjuvant endocrine therapy.

Non-limiting examples of endocrine therapy include, but are not limited to, Anastrozole (Arimidex), Exemestane (Aromasin), Fulvestrant (Faslodex), Goserelin (Zoladex), Letrozole (Femara), Leuprolide acetate (Eligard, Fensolvi, Lupron), Megestrol (Megace ES), Tamoxifen (Nolvadex, Soltamox), Toremifene (Fareston).

In some aspects, a subject in need thereof has relapsed breast cancer, wherein the subject has been previously treated with at least one endocrine therapy in combination with at least one CDK4/6 inhibitor. In some aspects, the subject can have been treated with the at least one endocrine therapy in combination with the at least one CDK4/6 inhibitor for at least about 12 months.

Non-limiting examples of CDK4/6 inhibitors include, but are not limited to, palbociclib, abemaciclib, ribociclib, trilaciclib, SHR-6390, FCN-437c, lerociclib, milciclib, PF-06873600, XZP-3287, zotiraciclib, BEBT-209, BPI-16350, CS-3002, fadraciclib, HS-10342, ON-123300, PF-06842874, TQ-05510, BPI-1178, JS-101, NUV-422, AU-294, CCT-68127, ETH-155008, HEC-80797, JRP-890, JS-104, NEOS-518, PF-07104091, PF-07220060, RMC-4550, SRX-3177, VS-2370, VS-2370, or pharmaceutically acceptable salts thereof.

Accordingly, in some aspects, a subject in need thereof is a postmenopausal woman having relapsed HR+HER2− breast cancer, wherein the subject in need thereof has at least one PIK3CA mutation, wherein the subject has been previously treated with at least one endocrine therapy in combination with at least one CDK4/6 inhibitor. Accordingly, in some aspects, a subject in need thereof is a postmenopausal woman having relapsed HR+HER2− breast cancer, wherein the subject in need thereof has at least one PIK3CA mutation, wherein the subject has been previously treated with neoadjuvant and/or adjuvant endocrine therapy and the relapsed breast cancer is characterized by progressive disease more than about 12 months from the completion of the neoadjuvant and/or adjuvant endocrine therapy, wherein the subject has been further treated with a combination of at least one endocrine therapy in combination with at least one CDK4/6 inhibitor. In some aspects, the further treatment with a combination of at least one endocrine therapy in combination with at least one CDK4/6 inhibitor can have been administered to the subject for at least about 12 months.

In some aspects, a subject in need thereof has breast cancer that is identified to be progressive while the subject is being treated with at least one endocrine therapy in combination with at least one CDK4/6 inhibitor. Accordingly, in some aspects, a subject in need thereof is a postmenopausal woman having HR+HER2− breast cancer that is identified to be progressive while the subject is being treated with at least one endocrine therapy in combination with at least one CDK4/6 inhibitor, wherein the subject in need thereof has at least one PIK3CA mutation. In some aspects, the subject can have been treated with the at least one endocrine therapy in combination with the at least one CDK4/6 inhibitor for at least about 12 months.

In some aspects, a subject in need thereof has breast cancer that is identified to be progressive after the subject has been treated with at least one endocrine therapy in combination with at least one CDK4/6 inhibitor. Accordingly, in some aspects, a subject in need thereof is a postmenopausal woman having HR+HER2− breast cancer that is identified to be progressive after the subject has been treated with at least one endocrine therapy in combination with at least one CDK4/6 inhibitor, wherein the subject in need thereof has at least one PIK3CA mutation. In some aspects, the subject can have been treated with the at least one endocrine therapy in combination with the at least one CDK4/6 inhibitor for at least about 12 months.

In some aspects, a subject in need thereof is a subject that is at risk of hyperglycemia. In some aspects, a subject that is at risk of hyperglycemia has HbA1c levels of about 5.7% to about 6.4%. In some aspects, a subject that is at risk of hyperglycemia has HbA1c levels of about 5.5% to about 6.4%. In some aspects, a subject that is at risk of hyperglycemia has a fasting plasma glucose (FPG) level of greater than about 100 mg/dL (5.6 mmol/L) and less than about 140 mg/dL (7.7 mmol/L). In some aspects, a subject that is at risk of hyperglycemia has HbA1c levels of about 5.7% to about 6.4% and a fasting plasma glucose (FPG) level of greater than about 100 mg/dL (5.6 mmol/L) and less than about 140 mg/dL (7.7 mmol/L). In some aspects, a subject that is at risk of hyperglycemia has HbA1c levels of about 5.5% to about 6.4% and a fasting plasma glucose (FPG) level of greater than about 100 mg/dL (5.6 mmol/L) and less than about 140 mg/dL (7.7 mmol/L). In some aspects, a subject that is at risk of hyperglycemia has HbA1c levels of about 5.7% to about 6.4% and a fasting plasma glucose (FPG) level of less than about 140 mg/dL (7.7 mmol/L). In some aspects, a subject that is at risk of hyperglycemia has HbA1c levels of about 5.5% to about 6.4% and a fasting plasma glucose (FPG) level of less than about 140 mg/dL (7.7 mmol/L). In some aspects, a subject that is at risk of hyperglycemia has HbA1c levels of greater than about 5.5%. In some aspects, a subject that is at risk of hyperglycemia has HbA1c levels of greater than or equal to about 5.5%. In some aspects, a subject that is at risk of hyperglycemia has HbA1c levels of greater than about 5.6%. In some aspects, a subject that is at risk of hyperglycemia has a fasting glucose level that is greater than about 100 mg/dL. In some aspects, a subject that is at risk of hyperglycemia has HbA1c levels of greater than about 5.5% and/or a fasting glucose level that is greater than about 100 mg/dL. In some aspects, a subject that is at risk of hyperglycemia has HbA1c levels of about 39 mmol/mol to about 47 mmol/mol.

In some aspects, a subject has an HbA1c level of greater than about 5.5%.

In some aspects, a subject that is at risk of hyperglycemia has a Homeostasis Model Assessment of Insulin Resistance (HOMA-IR) score of greater than about 1.8. As would be appreciated by the skilled artisan, the HOMA-IR score is calculated as follows: fasting serum insulin (μU/ml)×fasting plasma glucose (mmol/L)/22.5 (see Matthews et al. Diabetologia, 1985, 28(7), which is incorporated herein by reference in its entirety for all purposes).

In some aspects, a subject in need thereof has a body mass index (BMI) of greater than about 20 kg/m². In some aspects a subject in need thereof has a body mass index (BMI) of greater than or equal to about 30 kg/m².

In some aspects, a subject in need thereof has triple-negative metastatic breast cancer and at least one of: i) a BMI of greater than or equal to about 30 kg/m²; and ii) an HbA1c level of greater about 5.5%. In some aspects, the subject has previously received at least one line of treatment for the metastatic breast cancer. In some aspects, the subject has previously received at least two lines of treatment for the metastatic breast cancer.

In some aspects, a subject in need thereof has at least one metabolic dysfunction. In some aspects, the at least one metabolic dysfunction can be excessive visceral adiposity, dyslipidemia, obesity (BMI≥30), elevated leptin levels, depressed adiponectin levels, high leptin-to-adiponectin ratio, elevated fasting insulin levels, elevated fasting insulin levels accompanied by chronic inflammation, insulin resistance, high fasting glucose, elevated HbA1c, or any combination thereof.

### Dosing Schedules

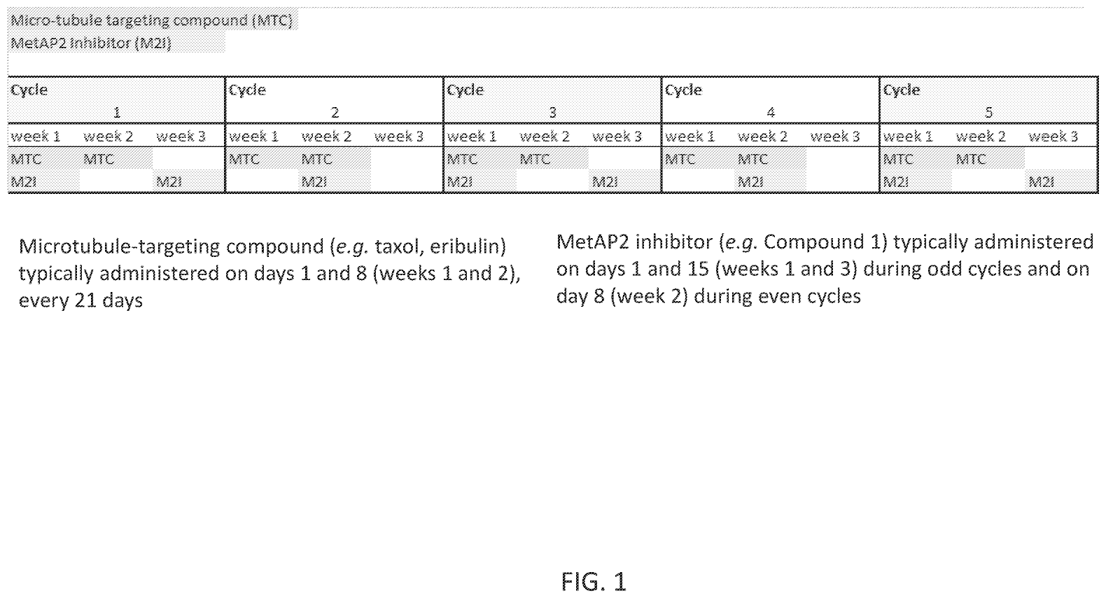

In some aspects of the methods of the present disclosure wherein the subject is to be administered a combination of a MetAP2 inhibitor and a microtubule-targeting compounds, the MetAP2 inhibitor can be administered to the subject every 14 days (Q14D) and the microtubule-targeting compound can be administered on days 1 and 8 of a 21-day cycle. Said alternatively, the microtubule-targeting compound can be administered on days 1 and 8 of a 21-day cycle, and the MetAP2 inhibitor can be administered on days 1 and 15 of odd cycles (e.g. the first cycle, the third cycle, the fifth cycle, etc.) and on Day 8 of the even cycles (e.g. the second cycle, the fourth cycle, the sixth cycle, etc.). A schematic of this dosing schedule is shown in FIG. 1.

In some aspects of the methods of the present disclosure wherein the subject is to be administered a combination of a MetAP2 inhibitor and a microtubule-targeting compound, the MetAP2 inhibitor and the microtubule-targeting compound can be administered in a 21-day cycle that comprises administering to the subject both the MetAP2 inhibitor and the microtubule-targeting compound once every 7 days (Q7D) for two weeks, followed by one week of no administration of the MetAP2 inhibitor and the microtubule-targeting compound. Thus, in a non-limiting example, the MetAP2 inhibitor and the microtubule-targeting compound can be administered on days 1 and 8 of a 21-day cycle.

In some aspects of the methods of the present disclosure wherein the subject is to be administered a combination of a MetAP2 inhibitor and a microtubule-targeting compound, the MetAP2 inhibitor and the microtubule targeting compound can be administered in a 21-day cycle that comprises administering the MetAP2 inhibitor once every 21 days (Q21D) and administering the microtubule-targeting compound once every 7 days (Q7D) for two weeks, followed by one week of no administration of the microtubule targeting compound. Thus, in a non-limiting example, the MetAP2 inhibitor can be administered on day 1 of a 21-day cycle and the microtubule-targeting compound can be administered on days 1 and 8 of said 21-day cycle. In another non-limiting example, the MetAP2 inhibitor can be administered at least once during the 21-day cycle and the microtubule-targeting compound can be administered on days 1 and 8 of the 21-day cycle.

In some aspects of the methods of the present disclosure wherein the subject is to be administered a combination of a MetAP2 inhibitor and a microtubule-targeting compounds, the MetAP2 inhibitor can be administered to the subject every 14 days (Q14D) and the microtubule-targeting compound can be administered on day 1 and one day between days 5 and 11 (inclusive) of a 21-day cycle. Said alternatively, the microtubule-targeting compound can be administered on day 1 and one day between days 5 and 11 (inclusive) of a 21-day cycle of a 21-day cycle, and the MetAP2 inhibitor can be administered on days 1 and 15 of odd cycles (e.g. the first cycle, the third cycle, the fifth cycle, etc.) and on Day 8 of the even cycles (e.g. the second cycle, the fourth cycle, the sixth cycle, etc.).

In some aspects of the methods of the present disclosure wherein the subject is to be administered a combination of a MetAP2 inhibitor, a SERD and a PI3K inhibitor, the MetAP2 inhibitor and the SERD inhibitor can be administered to the subject over the course of a 14-day pretreatment phase, and then the administration of the PI3K inhibitor can start on day 15.

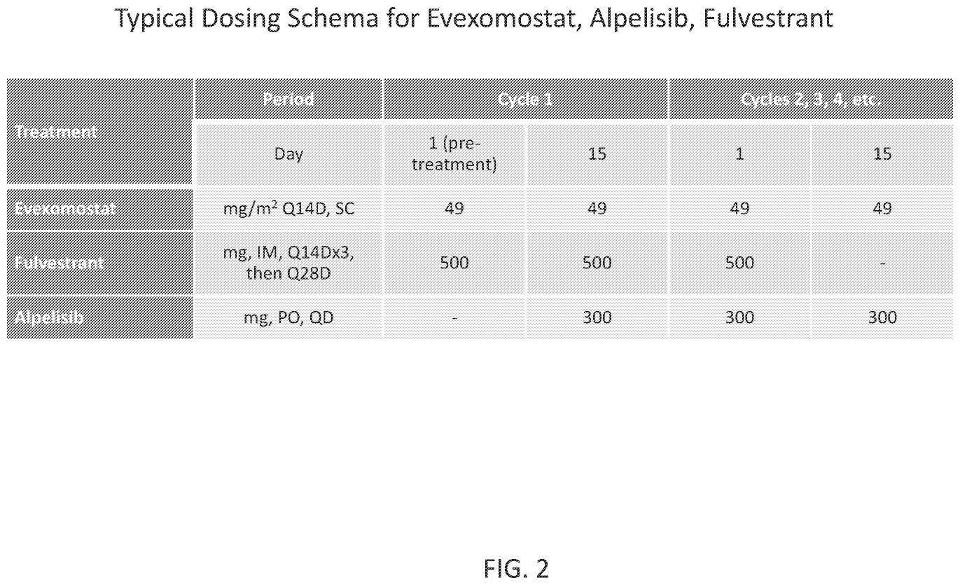

In some aspects of the methods of the present disclosure wherein the subject is to be administered a combination of a MetAP2 inhibitor, a PI3K inhibitor and a SERD, the MetAP2 inhibitor can be administered to the subject every 14 days (Q14D) starting on Day 1 of a 28-day cycle, the SERD can be administered to the subject every 14 days (Q14D) starting on Day 1 of the 28-day cycle, and the PI3K inhibitor can be administered once a day (QD) starting on day 15 of the cycle. Said alternatively, the MetAP2 inhibitor can be administered on days 1 and 15 of the 28-day cycle, the SERD can be administered on days 1 and 15 of the 28-day cycle, and the PI3K inhibitor can be administered on days 15-28 of the 28-day cycle. After the completion of one, 28-day cycle of the preceding triple combination therapy, subsequent 28-day cycles can be performed wherein the MetAP2 inhibitor is administered to the subject every 14 days (Q14D) starting on Day 1 of a new, 28-day cycle, the SERD is administered once every 28 days (Q28D) starting on Day 1 of the new, 28-day cycle, and the alpelisib is administered once a day (QD) starting on day 1 of the new, 28-day cycle. Said alternatively, in second and subsequent cycles, the MetAP2 inhibitor can be administered on days 1 and 15 of the 28-day cycle, the SERD can be administered on day 1 of the 28-day cycle, and alpelisib can be administered on days 1-28 of the 28-day cycle. A schematic of these dosing schedules in shown in FIG. 2.

In some aspects of the methods of the present disclosure wherein the subject is to be administered a combination of a MetAP2 inhibitor, a PI3K inhibitor and a SERD, the MetAP2 inhibitor can be administered to the subject every 14 days (Q14D) starting on Day 1 of a 28-day cycle, the SERD can be administered to the subject every 14 days (Q14D) starting on Day 1 of the 28-day cycle, and the PI3K inhibitor can be administered once a day (QD) starting on day 1 of the cycle. Said alternatively, the MetAP2 inhibitor can be administered on days 1 and 15 of the 28-day cycle, the SERD can be administered on days 1 and 15 of the 28-day cycle, and the PI3K inhibitor can be administered on days 1-28 of the 28-day cycle. After the completion of one, 28-day cycle of the preceding triple combination therapy, subsequent 28-day cycles can be performed wherein the MetAP2 inhibitor is administered to the subject every 14 days (Q14D) starting on Day 1 of a new, 28-day cycle, the SERD is administered once every 28 days (Q28D) starting on Day 1 of the new, 28-day cycle, and the alpelisib is administered once a day (QD) starting on day 1 of the new, 28-day cycle. Said alternatively, in second and subsequent cycles, the MetAP2 inhibitor can be administered on days 1 and 15 of the 28-day cycle, the SERD can be administered on day 1 of the 28-day cycle, and alpelisib can be administered on days 1-28 of the 28-day cycle. A schematic of these dosing schedules in shown in FIG. 2.

### General Definitions

Certain compounds of the present disclosure may exist in particular geometric or stereoisomeric forms. The present disclosure contemplates all such compounds, including cis- and trans-isomers, R- and S-enantiomers, diastereomers, (D)-isomers, (L)-isomers, the racemic mixtures thereof, and other mixtures thereof, as falling within the scope of the disclosure. Additional asymmetric carbon atoms may be present in a substituent such as an alkyl group. All such isomers, as well as mixtures thereof, are intended to be included in this disclosure. Any representation of a particular isomer is merely exemplary (e.g., the exemplification of a trans-isomer, also encompasses a cis-isomer).

If, for instance, a particular enantiomer of a compound of the present disclosure is desired, it may be prepared by asymmetric synthesis or by derivation with a chiral auxiliary, where the resulting diastereomeric mixture is separated and the auxiliary group cleaved to provide the pure desired enantiomer. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomer.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like. In addition, a crystal polymorphism may be present for the compounds represented by the formula. It is noted that any crystal form, crystal form mixture, or anhydride or hydrate thereof is included in the scope of the present disclosure. Furthermore, so-called metabolite which is produced by degradation of the present compound in vivo is included in the scope of the present disclosure.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four nonidentical substituents is termed a “chiral center”.

“Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures and other compounds discussed in this disclosure include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-enamine.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure, and are substituted with various groups as described herein.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

A woman can be defined as “postmenopausal” using any of the following criteria: i) ≥45 years of age and has not had menses for >2 years; ii) Amenorrheic for >2 years without a hysterectomy and oophorectomy and a follicle-stimulating hormone value in the postmenopausal range upon evaluation; and/or iii) post hysterectomy with oophorectomy.

As used herein, the term “temporal proximity” refers to that administration of one therapeutic agent (e.g., a MetAP2 inhibitor compound disclosed herein) occurs within a time period before or after the administration of another therapeutic agent (e.g., palbociclib), such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

The terms “effective amount” and “therapeutically effective amount” of an agent or compound are used in the broadest sense to refer to a nontoxic but sufficient amount of an active agent or compound to provide the desired effect or benefit.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “combination therapy” or “co-therapy” includes the administration of a compound of the present disclosure, or a pharmaceutically acceptable salt, polymorph or solvate thereof, and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents.

It is to be understood that the present disclosure also provides pharmaceutical compositions comprising any compound described herein in combination with at least one pharmaceutically acceptable excipient or carrier.

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

### Exemplary Embodiments

Embodiment 1. A combination comprising at least one MetAP2 inhibitor, or a pharmaceutically acceptable salt thereof, and eribulin, or a pharmaceutically acceptable salt thereof, for use in treating a cancer in a subject.

Embodiment 2. A method of treating cancer in a subject in need thereof, the method comprising administering to the subject at least one therapeutically effective amount of at least one MetAP2 inhibitor, or a pharmaceutically acceptable salt thereof, and at least one therapeutically effective amount of eribulin, or a pharmaceutically acceptable salt thereof.

Embodiment 3. A MetAP2 inhibitor, or a pharmaceutically acceptable salt thereof, for use in a method of treating a cancer in a subject, wherein the method further comprises administration of eribulin, or a pharmaceutically acceptable salt thereof.

Embodiment 4. Eribulin, or a pharmaceutically acceptable salt thereof, for use in a method of treating a cancer in a subject, wherein the method further comprises administration of at least one MetAP2 inhibitor or a pharmaceutically acceptable salt thereof.

Embodiment 5. The combination for use of embodiment 1, the method of embodiment 2, the MetAP2 inhibitor for use of embodiment 3, or the eribulin for use of embodiment 4, wherein the at least one MetAP2 inhibitor, or pharmaceutically acceptable salt thereof, and the eribulin, or pharmaceutically acceptable salt thereof, are administered concurrently or in temporal proximity.

Embodiment 6. The combination for use of embodiment 1 or 5, the method of embodiment 2 or 5, the MetAP2 inhibitor for use of embodiment 3 or 5, or the eribulin for use of embodiment 4 or 5, wherein the MetAP2 inhibitor is selected from:

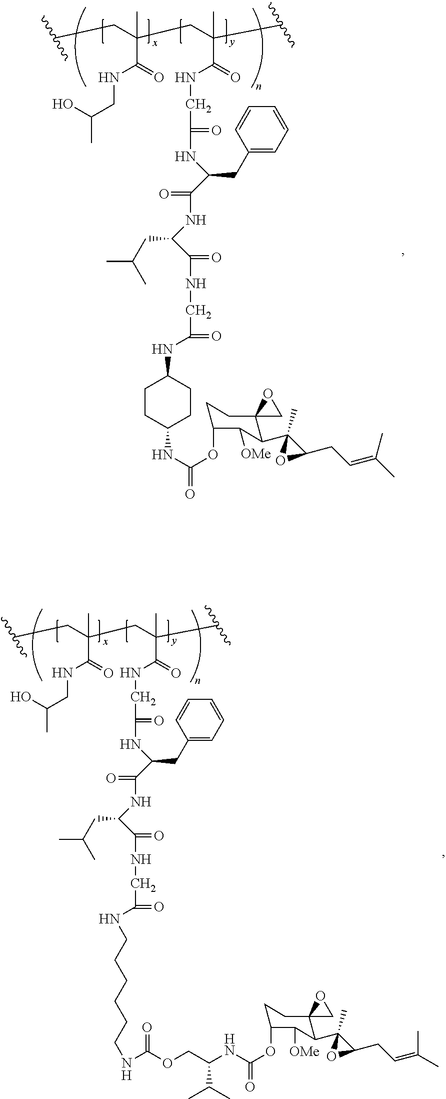

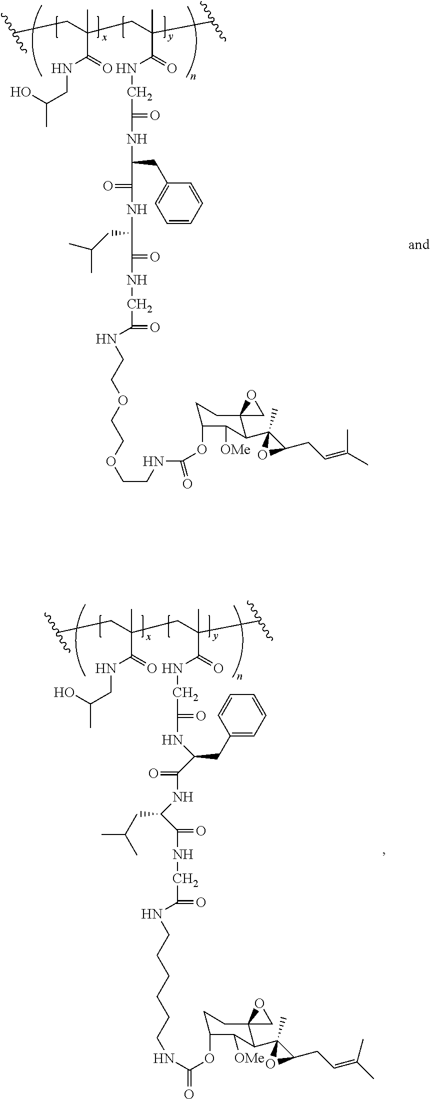

or a pharmaceutically acceptable salt thereof,
wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

Embodiment 7. The combination for use of embodiment 1 or 5-6, the method of embodiment 2 or 5-6, the MetAP2 inhibitor for use of embodiment 3 or 5-6, or the eribulin for use of embodiment 4 or 5-6, wherein the MetAP2 inhibitor is:

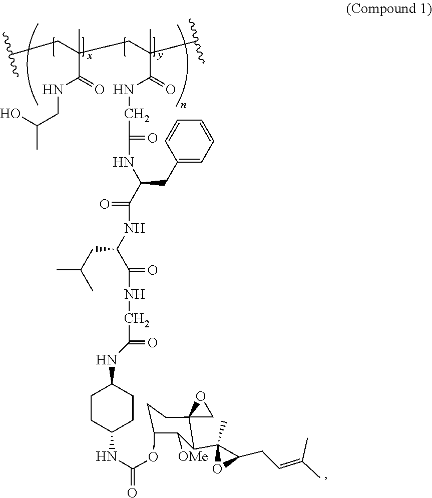

or a pharmaceutically acceptable salt thereof, wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

Embodiment 8. The combination for use of embodiment 1 or 5-7, the method of embodiment 2 or 5-7, the MetAP2 inhibitor for use of embodiment 3 or 5-7, or the eribulin for use of embodiment 4 or 5-7, wherein the eribulin is eribulin mesylate.

Embodiment 9. The combination for use of embodiment 1 or 5-8, the method of embodiment 2 or 5-8, the MetAP2 inhibitor for use of embodiment 3 or 5-8, or the eribulin for use of embodiment 4 or 5-8, wherein the eribulin or the eribulin mesylate is administered/for administration to the subject in an amount of:
i) about 1.4 mg/m²;
ii) about 1.1 mg/m² or
iii) about 0.7 mg/m², preferably wherein the eribulin or the eribulin mesylate is administered/for administration intravenously.

Embodiment 10. The combination for use of embodiment 1 or 5-9, the method of embodiment 2 or 5-9, the MetAP2 inhibitor for use of embodiment 3 or 5-9, or the eribulin for use of embodiment 4 or 5-9, wherein the MetAP2 inhibitor, or pharmaceutically acceptable salt thereof, is administered/for administration to the subject in an amount of:
i) about 49 mg/m²;
ii) about 36 mg/m²; or
iii) about 65 mg/m²;
preferably wherein the MetAP2 inhibitor, or pharmaceutically acceptable salt thereof, is administered/for administration subcutaneously.

Embodiment 11. The combination for use of embodiment 1 or 5-10, the method of embodiment 2 or 5-10, the MetAP2 inhibitor for use of embodiment 3 or 5-10, or the eribulin for use of embodiment 4 or 5-10, wherein the MetAP2 inhibitor is administered/for administration once every 14 days (Q14D).

Embodiment 12. The combination for use of embodiment 1 or 5-11, the method of embodiment 2 or 5-11, the MetAP2 inhibitor for use of embodiment 3 or 5-11, or the eribulin for use of embodiment 4 or 5-11, wherein the eribulin or eribulin mesylate is administered/for administration on day 1 and one day between days 5 and 11 (inclusive) of a 21-day cycle, preferably wherein the eribulin or eribulin mesylate is administered/for administration on day 1 and day 8 of a 21-day cycle.

Embodiment 13. The combination for use of embodiment 1 or 5-12, the method of embodiment 2 or 5-10, the MetAP2 inhibitor for use of embodiment 3 or 5-12, or the eribulin for use of embodiment 4 or 5-12, wherein the eribulin or eribulin mesylate and the MetAP2 inhibitor, or pharmaceutically acceptable salt thereof, is administered/for administration to the subject in a first 21-day cycle, followed by a second 21-day cycle,
wherein the first 21-day cycle comprises:
i) administering the MetAP2 inhibitor, or pharmaceutically acceptable salt thereof, on days 1 and 15 of the first 21-day cycle; and
ii) administering the eribulin or eribulin mesylate on day 1 and one day between days 5 and 11 (inclusive), preferably day 8, of the first 21-day cycle;
wherein the second 21-day cycle comprises:
i) administering the MetAP2 inhibitor, or pharmaceutically acceptable salt thereof, on day 8 of the second 21-day cycle; and
ii) administering the eribulin or eribulin mesylate on day 1 and one day between days 5 and 11 (inclusive), preferably day 8, of the second 21-day cycle.

Embodiment 14. The combination for use of embodiment 13, the method of embodiment 12, the MetAP2 inhibitor for use of embodiment 13, or the eribulin for use of embodiment 13, wherein the first 21-day cycle and the second 21 day cycle are repeated: i) at least once, for a total of at least four, 21-day cycles;
ii) at least twice, for a total of at least six, 21-day cycles;
iii) at least three times, for a total of at least eight, 21-day cycles;
iv) at least four times, for a total of at least ten, 21-day cycles; or
v) at least five times, for a total of at least twelve, 21-day cycles.

Embodiment 15. The combination for use of embodiment 1 or 5-14, the method of embodiment 2 or 5-14, the MetAP2 inhibitor for use of embodiment 3 or 5-14, or the eribulin for use of embodiment 4 or 5-14, wherein the subject has breast cancer, preferably wherein the breast cancer is triple-negative beast cancer, preferably wherein the triple-negative breast cancer is metastatic.

Embodiment 16. The combination for use of embodiment 1 or 5-15, the method of embodiment 2 or 5-15, the MetAP2 inhibitor for use of embodiment 3 or 5-15, or the eribulin for use of embodiment 4 or 5-15, wherein the subject has at least one of:
i) a BMI of greater than or equal to about 30 kg/m²; and
ii) an HbA1c level of greater about 5.5%.

Embodiment 17. The combination for use of embodiment 1 or 5-16, the method of embodiment 2 or 5-16, the MetAP2 inhibitor for use of embodiment 3 or 5-16, or the eribulin for use of embodiment 4 or 5-16, wherein the subject has previously received at least one line of treatment for the cancer.

Embodiment 18. A combination comprising at least one MetAP2 inhibitor, or a pharmaceutically acceptable salt thereof, fulvestrant, or a pharmaceutically acceptable salt thereof, and alpelisib, or a pharmaceutically acceptable salt thereof, for use in treating a cancer in a subject.

Embodiment 19. A method of treating cancer in a subject in need thereof, the method comprising administering to the subject at least one therapeutically effective amount of at least one MetAP2 inhibitor, or a pharmaceutically acceptable salt thereof, at least one therapeutically effective amount of fulvestrant, or a pharmaceutically acceptable salt thereof, and at least one therapeutically effective amount of alpelisib, or a pharmaceutically acceptable salt thereof.

Embodiment 20. A MetAP2 inhibitor, or a pharmaceutically acceptable salt thereof, for use in a method of treating a cancer in a subject, wherein the method further comprises administration of fulvestrant, or a pharmaceutically acceptable salt thereof, and alpelisib, or a pharmaceutically acceptable salt thereof.

Embodiment 21. Fulvestrant, or a pharmaceutically acceptable salt thereof, for use in a method of treating a cancer in a subject, wherein the method further comprises administration of at least one MetAP2 inhibitor, or a pharmaceutically acceptable salt thereof, and alpelisib, or a pharmaceutically acceptable salt thereof.

Embodiment 22. Alpelisib, or a pharmaceutically acceptable salt thereof, for use in a method of treating a cancer in a subject, wherein the method further comprises administration of at least one MetAP2 inhibitor, or a pharmaceutically acceptable salt thereof, and fulvestrant, or a pharmaceutically acceptable salt thereof.

Embodiment 23. The combination for use of embodiment 18, the method of embodiment 19, the MetAP2 inhibitor for use of embodiment 20, the fulvestrant for use of embodiment 21, or the alpelisib for use of embodiment 22, wherein the at least one MetAP2 inhibitor, or pharmaceutically acceptable salt thereof, the alpelisib, or pharmaceutically acceptable salt thereof, and the fulvestrant, or pharmaceutically acceptable salt thereof, are administered concurrently or in temporal proximity.

Embodiment 24. The combination for use of embodiment 18 or 22, the method of embodiment 19 or 22, the MetAP2 inhibitor for use of embodiment 20 or 22, the fulvestrant for use of embodiment 21 or 22, or the alpelisib for use of embodiment 22 or 22, wherein the MetAP2 inhibitor is selected from:

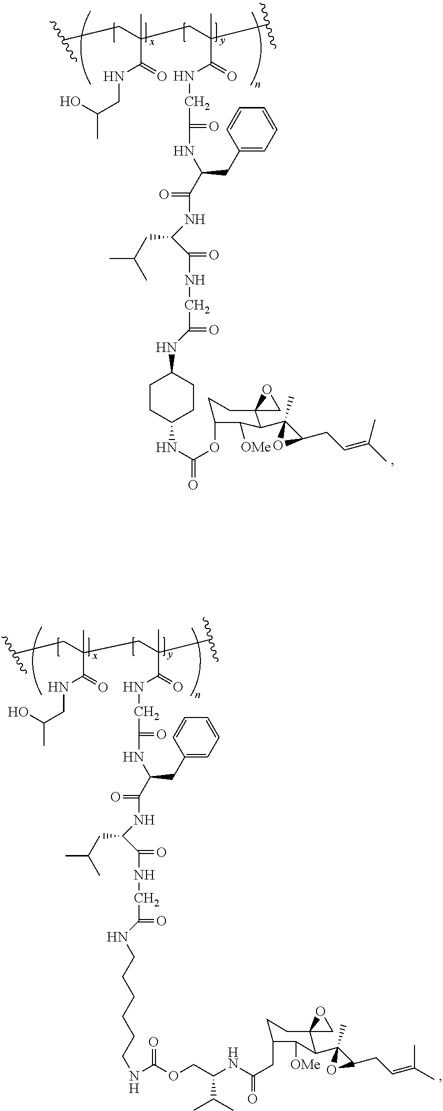

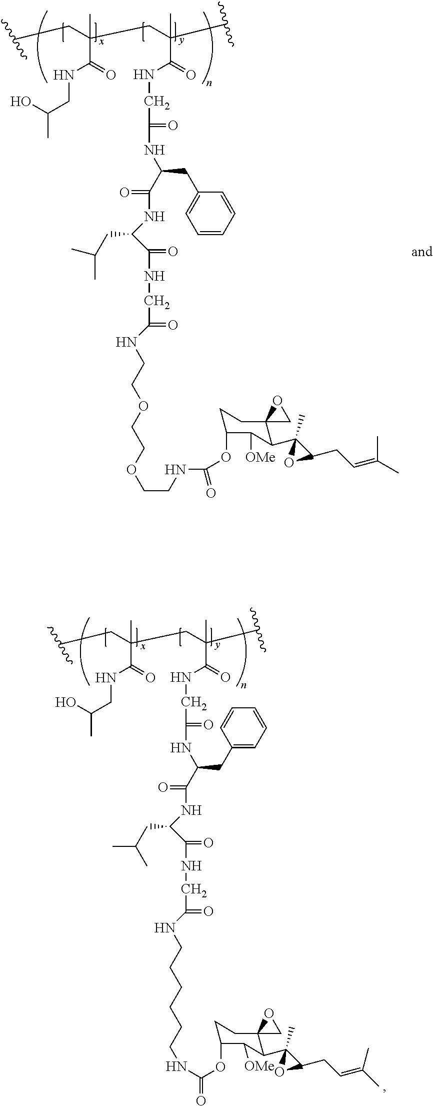

or a pharmaceutically acceptable salt thereof,
wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

Embodiment 25. The combination for use of embodiment 18 or 23-24, the method of embodiment 19 or 23-24, the MetAP2 inhibitor for use of embodiment 20 or 23-24, the fulvestrant for use of embodiment 21 or 23-24, or the alpelisib for use of embodiment 22 or 23-24, wherein the MetAP2 inhibitor is:

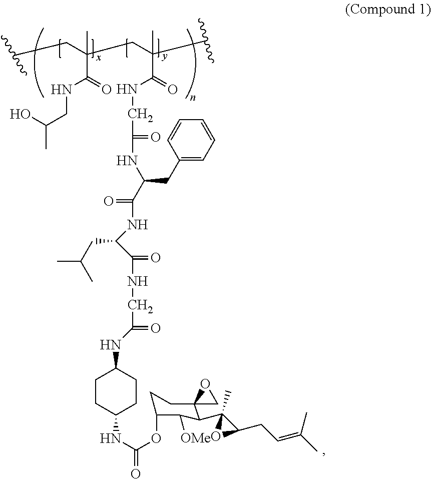

wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

Embodiment 26. The combination for use of embodiment 18 or 23-25, the method of embodiment 19 or 23-25, the MetAP2 inhibitor for use of embodiment 20 or 23-25, the fulvestrant for use of embodiment 21 or 23-25, or the alpelisib for use of embodiment 22 or 23-25, wherein the alpelisib, or pharmaceutically acceptable salt thereof, is administered/for administration to the subject in an amount of:
i) about 300 mg;
ii) about 250 mg; or
iii) about 200 mg,
preferably wherein the alpelisib, or pharmaceutically acceptable salt thereof, is administered/for administration orally.

Embodiment 27. The combination for use of embodiment 18 or 23-26, the method of embodiment 19 or 23-26, the MetAP2 inhibitor for use of embodiment 20 or 23-26, the fulvestrant for use of embodiment 21 or 23-26, or the alpelisib for use of embodiment 22 or 23-26, wherein the fulvestrant, or pharmaceutically acceptable salt thereof, is administered/for administration to the subject in an amount of:
i) about 500 mg; or
ii) about 250 mg;
preferably wherein the fulvestrant, or pharmaceutically acceptable salt thereof, is administered/for administration intramuscularly.

Embodiment 28. The combination for use of embodiment 18 or 23-27, the method of embodiment 19 or 23-27, the MetAP2 inhibitor for use of embodiment 20 or 23-27, the fulvestrant for use of embodiment 21 or 23-27, or the alpelisib for use of embodiment 22 or 23-27, wherein the MetAP2 inhibitor, or pharmaceutically acceptable salt thereof, is administered/for administration to the subject in an amount of:
i) about 49 mg/m²;
ii) about 36 mg/m²;
iii) about 65 mg/m²; or
iv) about 27 mg/m²
preferably wherein the MetAP2 inhibitor, or pharmaceutically acceptable salt thereof, is administered/for administration subcutaneously.

Embodiment 29. The combination for use of embodiment 18 or 23-28, the method of embodiment 19 or 23-28, the MetAP2 inhibitor for use of embodiment 20 or 23-28, the fulvestrant for use of embodiment 21 or 23-28, or the alpelisib for use of embodiment 22 or 23-28, wherein the MetAP2 inhibitor is administered/for administration once every 14 days (Q14D).

Embodiment 30. The combination for use of embodiment 18 or 23-29, the method of embodiment 19 or 23-29, the MetAP2 inhibitor for use of embodiment 20 or 23-29, the fulvestrant for use of embodiment 21 or 23-29, or the alpelisib for use of embodiment 22 or 23-29, wherein the alpelisib inhibitor is administered/for administration once daily (QD).

Embodiment 31. The combination for use of embodiment 18 or 23-30, the method of embodiment 19 or 23-30, the MetAP2 inhibitor for use of embodiment 20 or 23-30, the fulvestrant for use of embodiment 21 or 23-30, or the alpelisib for use of embodiment 22 or 23-30, wherein the fulvestrant is administered/for administration once every 14 days (Q14D), wherein following the third administration of fulvestrant, the fulvestrant is administered/for administration once every 28 days (Q28D).

Embodiment 32. The combination for use of embodiment 18 or 23-31, the method of embodiment 19 or 23-31, the MetAP2 inhibitor for use of embodiment 20 or 23-31, the fulvestrant for use of embodiment 21 or 23-31, or the alpelisib for use of embodiment 22 or 23-31, wherein the MetAP2 inhibitor, or pharmaceutically acceptable salt thereof, the alpelisib, or pharmaceutically acceptable salt thereof, and fulvestrant, or pharmaceutically acceptable salt thereof, is administered/for administration to the subject in a first 28-day, followed by a second 21-day cycle,
wherein the first 28-day cycle comprises:
i) administering the MetAP2 inhibitor, or pharmaceutically acceptable salt thereof, on days 1 and 15 of the first 28-day cycle;
ii) administering the fulvestrant, or pharmaceutically acceptable salt thereof, on days 1 and 15 of the first 28-day cycle; and
iii) administering the alpelisib, or pharmaceutically acceptable salt thereof, on each of days 15-28 of the first 28-day cycle;
wherein the second 21-day cycle comprises.
i) administering the MetAP2 inhibitor, or pharmaceutically acceptable salt thereof, on days 1 and 15 of the second 28-day cycle; and
ii) administering the fulvestrant, or pharmaceutically acceptable salt thereof, on day 1 of the second 28-day cycle; and
iii) administering the alpelisib, or pharmaceutically acceptable salt thereof, on each of days 1-28 of the second 28-day cycle.

Embodiment 33. The combination for use of embodiment 18 or 23-32, the method of embodiment 19 or 23-32, the MetAP2 inhibitor for use of embodiment 20 or 23-32, the fulvestrant for use of embodiment 21 or 23-32, or the alpelisib for use of embodiment 22 or 23-32, wherein the second 28-day cycle is repeated
i) at least once, for a total of at least three, 28-day cycles;
ii) at least twice, for a total of at least four, 28-day cycles;
iii) at least three times, for a total of at least five, 28-day cycles;
iv) at least four times, for a total of at least six, 28-day cycles; or
v) at least five times, for a total of at least seven, 28-day cycles.

Embodiment 34. The combination for use of embodiment 18 or 23-33, the method of embodiment 19 or 23-33, the MetAP2 inhibitor for use of embodiment 20 or 23-33, the fulvestrant for use of embodiment 21 or 23-33, or the alpelisib for use of embodiment 22 or 23-33, wherein the subject has breast cancer, preferably wherein the breast cancer is HR+HER2− breast cancer, preferably wherein the breast cancer is relapsed breast cancer.

Embodiment 35. The combination for use of embodiment 18 or 23-34, the method of embodiment 19 or 23-34, the MetAP2 inhibitor for use of embodiment 20 or 23-34, the fulvestrant for use of embodiment 21 or 23-34, or the alpelisib for use of embodiment 22 or 23-34, wherein the subject hast at least one PIK3CA mutation.

Embodiment 36. The combination for use of embodiment 18 or 23-35, the method of embodiment 19 or 23-35, the MetAP2 inhibitor for use of embodiment 20 or 23-35, the fulvestrant for use of embodiment 21 or 23-35, or the alpelisib for use of embodiment 22 or 23-35, wherein the subject has been previously treated with neoadjuvant and/or adjuvant endocrine therapy, preferably wherein the breast cancer is characterized by progressive disease more than about 12 months from the completion of the neoadjuvant and/or adjuvant endocrine therapy

Embodiment 37. The combination for use of embodiment 18 or 23-36, the method of embodiment 19 or 23-36, the MetAP2 inhibitor for use of embodiment 20 or 23-36, the fulvestrant for use of embodiment 21 or 23-36, or the alpelisib for use of embodiment 22 or 23-36, wherein the subject has been previously treated with at least one endocrine therapy in combination with at least one CDK4/6 inhibitor, preferably wherein the subject has been treated with the at least one endocrine therapy in combination with the at least one CDK4/6 inhibitor for at least about 12 months.

Embodiment 38. The combination for use of embodiment 18 or 23-37, the method of embodiment 19 or 23-37, the MetAP2 inhibitor for use of embodiment 20 or 23-37, the fulvestrant for use of embodiment 21 or 23-37, or the alpelisib for use of embodiment 22 or 23-37, wherein the breast cancer is identified to be progressive while the subject is being treated or after the subject has been treated with at least one endocrine therapy in combination with at least one CDK4/6 inhibitor.

Embodiment 39. The combination for use of embodiment 18 or 23-38, the method of embodiment 19 or 23-38, the MetAP2 inhibitor for use of embodiment 20 or 23-38, the fulvestrant for use of embodiment 21 or 23-38, or the alpelisib for use of embodiment 22 or 22-31, wherein the subject is a postmenopausal woman.

Embodiment 40. The combination for use of embodiment 18 or 23-39, the method of embodiment 19 or 23-39, the MetAP2 inhibitor for use of embodiment 20 or 23-39, the fulvestrant for use of embodiment 21 or 23-39, or the alpelisib for use of embodiment 22 or 23-39, wherein the subject has at least one of:
i) HbA1c levels of about 5.7% to about 6.4%
ii) fasting plasma glucose (FPG) level of greater than about 100 mg/dL (5.6 mmol/L) and less than about 140 mg/dL (7.7 mmol/L);
iii) a body mass index (BMI) of greater than about 20 kg/m²
iv) a Homeostasis Model Assessment of Insulin Resistance (HOMA-IR) score of greater than about 1.8.

Embodiment 41. The combination for use, the method, the MetAP2 inhibitor for use, the eribulin for use, the fulvestrant for use, or the alpelisib for use of any one of the preceding embodiments, wherein the subject has at least one of:
i) a BMI of greater than or equal to about 30 kg/m², and
ii) an HbA1c level of greater than about 5.5%.

Embodiment 42. The combination for use, the method, the MetAP2 inhibitor for use, the eribulin for use, the fulvestrant for use, or the alpelisib for use of any one of the preceding embodiments, wherein the subject has at least one metabolic dysfunction, wherein the at least one metabolic dysfunction is excessive visceral adiposity, dyslipidemia, obesity (BMI≥30), elevated leptin levels, depressed adiponectin levels, high leptin-to-adiponectin ratio, elevated fasting insulin levels, elevated fasting insulin levels accompanied by chronic inflammation, insulin resistance, high fasting glucose, elevated HbA1c, or any combination thereof.

Embodiment 43. A combination comprising at least one MetAP2 inhibitor, or a pharmaceutically acceptable salt thereof, and inavolisib, or a pharmaceutically acceptable salt thereof, for use in treating a cancer in a subject.

Embodiment 44. A method of treating cancer in a subject in need thereof, the method comprising administering to the subject at least one therapeutically effective amount of at least one MetAP2 inhibitor, or a pharmaceutically acceptable salt thereof, and at least one therapeutically effective amount of inavolisib, or a pharmaceutically acceptable salt thereof.

Embodiment 45. A MetAP2 inhibitor, or a pharmaceutically acceptable salt thereof, for use in a method of treating a cancer in a subject, wherein the method further comprises administration of inavolisib, or a pharmaceutically acceptable salt thereof.

Embodiment 46. Inavolisib, or a pharmaceutically acceptable salt thereof, for use in a method of treating a cancer in a subject, wherein the method further comprises administration of at least one MetAP2 inhibitor or a pharmaceutically acceptable salt thereof.

Embodiment 47. The combination for use, the method, the MetAP2 inhibitor for use, or the inavolisib for use of any one of the preceding embodiments, wherein the at least one MetAP2 inhibitor, or pharmaceutically acceptable salt thereof, and the inavolisib, or pharmaceutically acceptable salt thereof, are administered concurrently or in temporal proximity.

Embodiment 48. The combination for use, the method, the MetAP2 inhibitor for use, or the inavolisib for use of any one of the preceding embodiments, wherein the MetAP2 inhibitor is selected from:

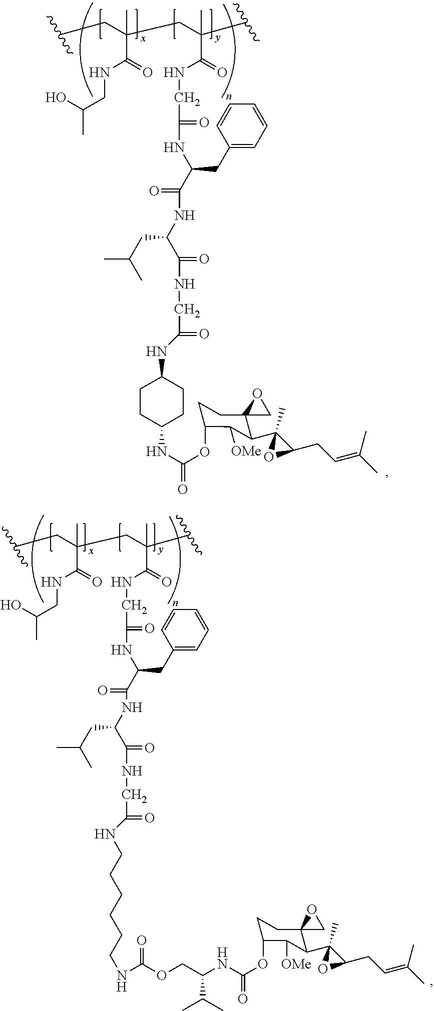

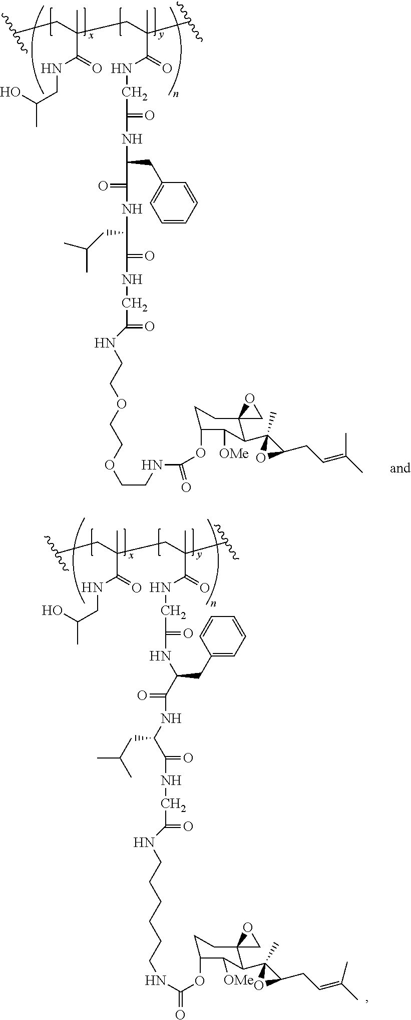

and or a pharmaceutically acceptable salt thereof,
wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

Embodiment 49. The combination for use, the method, the MetAP2 inhibitor for use, or the inavolisib for use of any one of the preceding embodiments, wherein the MetAP2 inhibitor is:

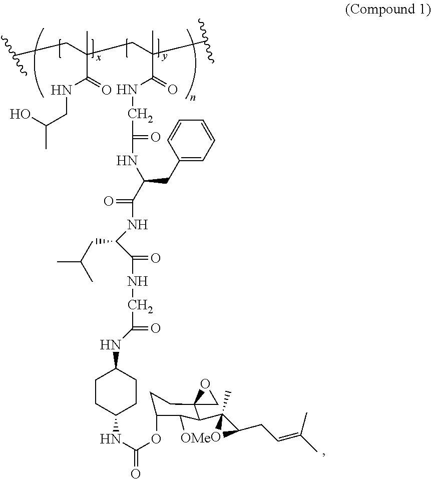

or a pharmaceutically acceptable salt thereof, wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

Embodiment 50. The combination for use, the method, the MetAP2 inhibitor for use, or the inavolisib for use of any one of the preceding embodiments, wherein the inavolisib is administered/for administration orally.

Embodiment 51. The combination for use, the method, the MetAP2 inhibitor for use, or the inavolisib for use of any one of the preceding embodiments, wherein the inavolisib is administered/for administration to the subject in an amount of:
i) about 3 mg;
ii) about 6 mg;
iii) about 9 mg; or
iv) about 12 mg.

Embodiment 52. The combination for use, the method, the MetAP2 inhibitor for use, or the inavolisib for use of any one of the preceding embodiments, wherein the MetAP2 inhibitor, or pharmaceutically acceptable salt thereof, is administered/for administration to the subject in an amount of:
i) about 49 mg/m²;
ii) about 36 mg/m²; or
iii) about 65 mg/m²;
preferably wherein the MetAP2 inhibitor, or pharmaceutically acceptable salt thereof, is administered/for administration subcutaneously.

Embodiment 53. The combination for use, the method, the MetAP2 inhibitor for use, or the inavolisib for use of any one of the preceding embodiments, wherein the MetAP2 inhibitor is administered/for administration once every 4 days (Q4D).

Embodiment 54. The combination for use, the method, the MetAP2 inhibitor for use, or the inavolisib for use of any one of the preceding embodiments, wherein the inavolisib is administered/for administration once daily.

Embodiment 55. The combination for use, the method, the MetAP2 inhibitor for use, or the inavolisib for use of any one of the preceding embodiments, wherein the subject has breast cancer, preferably wherein the breast cancer is triple-negative beast cancer, preferably wherein the triple-negative breast cancer is metastatic.

Embodiment 56. The combination for use, the method, the MetAP2 inhibitor for use, or the inavolisib for use of any one of the preceding embodiments, wherein the subject has at least one of:
i) a BMI of greater than or equal to about 30 kg/m²; and
ii) an HbA1c level of greater about 5.5%.

Embodiment 57. The combination for use, the method, the MetAP2 inhibitor for use, or the inavolisib for use of any one of the preceding embodiments, wherein the subject has previously received at least one line of treatment for the cancer.

Embodiment 58. The combination for use, the method, the MetAP2 inhibitor for use, or the inavolisib for use of any one of the preceding embodiments, wherein the subject has breast cancer, preferably wherein the breast cancer is HR+HER2− breast cancer, preferably wherein the breast cancer is relapsed breast cancer.

Embodiment 59. The combination for use, the method, the MetAP2 inhibitor for use, or the inavolisib for use of any one of the preceding embodiments, wherein the subject hast at least one PIK3CA mutation.

Embodiment 60. The combination for use, the method, the MetAP2 inhibitor for use, or the inavolisib for use, wherein the subject has at least one of:
i) a BMI of greater than or equal to about 30 kg/m²; and
ii) an HbA1c level of greater than about 5.5%.

Embodiment 61. The combination for use, the method, the MetAP2 inhibitor for use, or the inavolisib for use of any one of the preceding embodiments, wherein the subject has at least one metabolic dysfunction, wherein the at least one metabolic dysfunction is excessive visceral adiposity, dyslipidemia, obesity (BMI≥30), elevated leptin levels, depressed adiponectin levels, high leptin-to-adiponectin ratio, elevated fasting insulin levels, elevated fasting insulin levels accompanied by chronic inflammation, insulin resistance, high fasting glucose, elevated HbA1c, or any combination thereof.

### EXAMPLES

### Example 1—Phase 1b/2 Study of Compound 1 in Combination with Alpelisib and Fulvestrant in Postmenopausal Women at Risk for Hyperglycemia with Metastatic Breast Cancer and a PIK3CA Mutation after Treatment with CDK4/6 Inhibitor

The following non-limiting example is a description of a Phase 1b/2 study to determine the anti-tumor effects of a combination of Compound 1, alpelisib and fulvestrant in certain HR+HER2− breast cancer populations. Non-limiting goals of the study include characterizing the safety of the triplet drug combination (alpelisib, fulvestrant, and Compound 1), to test whether Compound 1, when given in combination with alpelisib and fulvestrant reduces the number and severity of hyperglycemic events and/or reduce the number of anti-diabetic medications needed to control the hyperglycemia for patients deemed at risk for alpelisib-induced hyperglycemia (baseline elevated HbA1c or well-controlled type 2 diabetes), and to assess preliminary anti-tumor efficacy and changes in key biomarkers and quality of life in the study population. The study population includes adult postmenopausal women with advanced or metastatic histologically and/or cytologically confirmed diagnosis of HR+, HER2− breast cancer and a PIK3CA mutation who have progressed on or following endocrine therapy plus a CDK4/6 inhibitor and who are at risk of hyperglycemia (i.e., HbA1c between 5.7 and 6.4% inclusive and/or HOMA-IR>1.8)

Up to 52 patients will be enrolled, starting with dose-escalation cohort(s) of 6 patients. Once the maximum tolerated dose (MTD) of the triplet therapy has been defined, additional patients will be enrolled until a total of up to 20 patients have completed at least two cycles of the triplet therapy at that dose. An additional 20 patients per new cohort may be enrolled at varying doses to further characterize the safety profile and/or anti-tumor effects of the triplet therapy and to identify an active biologic dose possibly lower than the maximum tolerated dose.

The planned escalation scheme starts at a Compound 1 dose of 36 mg/m² (one dose below the monotherapy MTD of 49 mg/m²) in combination with alpelisib and fulvestrant given at the marketed doses or adjusted doses, as per their respective labels. Based on aggregate safety data from the first two cycles of the first 6 patients, in the absence of ≥2 dose-limiting toxicities (DLTs), the Compound 1 dose may be increased for the next cohort to 49 mg/m². In the presence of ≥2 DLTs at the 36 mg/m² dose, the Compound 1 dose can be decreased to 27 mg/m² and the dose of alpelisib may also be adjusted if needed. The dose of fulvestrant will not be adjusted. If the Compound 1 dose of 49 mg/m² is determined not to be tolerable in combination with alpelisib and fulvestrant, then current and future patients will receive Compound 1 at 36 mg/m².

In the event of significantly low drug exposure of the active moiety of Compound 1 (C₂₄<200 pg/mL) at dosages of 49 mg/m², coupled with poor biomarker response (e.g., little/no change in insulin/leptin/adiponectin from baseline) and a favorable safety profile, an additional cohort of patients to be dosed with Compound 1 at 65 mg/m² may be enrolled, which would become the future dose in the combination if tolerated.

Patients will remain on study for up to 7 Cycles on the triplet therapy to characterize the safety and tolerability of the triplet therapy as well as to capture initial efficacy data (i.e., ORR and PFS following 6 months of the triplet therapy). Patients will be allowed to remain on the triplet therapy beyond the initial 7 Cycles if they are receiving clinical benefit, including stable disease, as determined by their treating oncologist.

The study will consist of a 14-day pre-treatment phase of Compound 1 plus fulvestrant starting on Cycle 1, Day 1 (C1D1) before adding alpelisib on C1D15.

Starting with the first cohort, patients will receive Compound 1 at 36 mg/m² administered subcutaneously and fulvestrant at 500 mg administered intramuscularly on day 1 of cycle 1 (C1D1). Starting on day 15 of cycle 1 (C1D15) and following fasted blood draws, Compound 1 will be administered at 36 mg/m² subcutaneously and fulvestrant will be administered at 500 mg intramuscularly, followed by alpelisib 300 mg orally (PO) once daily (QD) being added to the regimen (i.e., the ‘triplet therapy’).

In cycle 2 (C2) and beyond, all patients will receive triplet therapy with Compound 1 at 36 mg/m² subcutaneously biweekly (on C×D1 and C×D15)+fulvestrant 500 mg intramuscularly once every 28 days (C×D1)+alpelisib 300 mg daily orally.

As discussed above, in any of the cycles in the experiment, the dosage of Compound 1 can be increased to 49 mg/m² and 65 mg/m² or decreased to 27 mg/m² depending on pharmacokinetics and safety analysis.

### Outcome Measurements

Primary Outcome #1: test whether Compound 1, when given in combination with alpelisib and fulvestrant reduces the number and severity of hyperglycemic events and/or reduces the number of anti-diabetic medications needed to control the hyperglycemia for patients deemed at risk for alpelisib-induced hyperglycemia (baseline elevated HbA1c or well-controlled type 2 diabetes). The metric used to determine this outcome will be the measurement of fasting plasma glucose and the primary efficacy analysis will include the number and proportion of patients who experience Grade 3 or 4 hyperglycemia at least once during the first 4 Cycles of triplet therapy.

Primary Outcome #2: assess preliminary anti-tumor efficacy of the combination of alpelisib, fulvestrant and Compound 1. Tumor response will be determined using the RECIST v1.1 criteria and the objective response rate (ORR) consisting of CR and PR will be calculated. The ORR, CBR of CR plus PR plus SD of 24 weeks or greater from the start of study will be calculated.

Other prespecified outcome measurements: The number and type of anti-diabetic agents needed for glucose control, changes to key, fasting metabolic hormones (insulin, leptin, adiponectin), insulin resistance (using the HOMA-IR score) and HbA1c. The number and type of anti-diabetic agents needed for glucose control will be measured, along with changes to key, fasting metabolic hormones (insulin, leptin, adiponectin) FPG, insulin resistance (using the HOMA-IR score) and HbA1c as compared to baseline levels and on C5D15 and end of treatment. Circulating levels of glucose, insulin, HbA1C, leptin, adiponectin, Apelin, FGF21, and lipids will be measured in clinical chemistry laboratories. Other exploratory biomarkers, including angiogenic markers bFGF, VEGF (A, C and D), inflammatory markers IL-5, IL-6, hs-CRP, GM-CSF, Resistin, metastatic markers sFRP-1, sFRP-5, MetAP2 marker Cyclophilin A, as well as SHBG, and circulating tumor DNA (ctDNA) will be measured in clinical chemistry laboratories.

### Eligibility Criteria

1. Patient is an adult≥18 years old at the time of informed consent(s) and has signed informed consent(s) before any trial related activities and according to local guidelines.

2. Patient with histologically and/or cytologically confirmed diagnosis of HR+, HER2− breast cancer, as determined by the local laboratory.

3. Patient has identified PIK3CA mutation status using a FDA-approved test, as determined either during Screening or was previously determined to have the mutation as evidenced by written documentation.

4. Patient has advanced (local regionally recurrent not amenable to curative therapy or metastatic) breast cancer meeting any of the following categories:

Relapsed disease, with documented evidence of progressive disease (PD) more than 12 months from completion of (neo)adjuvant endocrine therapy and then subsequently progressed, with documented evidence of PD while receiving or after one line of endocrine therapy plus a CDK 4/6 inhibitor for at least 12 months for their metastatic disease.

Newly diagnosed advanced breast cancer, with relapsed disease (i.e., documented evidence of PD) while receiving or after only one line of endocrine therapy plus a CDK 4/6 inhibitor for at least 12 months.

Recurrent disease or PD while receiving or after aromatase inhibitor (AI) therapy (i.e., letrozole, anastrozole, exemestane) with co-treatment with a CDK 4/6 inhibitor for at least 12 months.

5. Patient has either measurable disease per the Response Evaluation Criteria in Solid Tumors (RECIST) v1.1, or at least one evaluable predominantly lytic bone lesion

6. Patient has an Eastern Cooperative Oncology Group Performance Status (ECOG-PS)≤1

7. Patient has a Screening fasting plasma glucose (FPG) level≤140 mg/dL (7.7 mmol/L) and an HbA1c of ≥5.7 and ≥6.4% (39 and 47 mmol/mol respectively). Both criteria must be met.

8. Patient has a body mass index (BMI)≥20 kg/m².

9. Patient is postmenopausal. Postmenopausal is defined as any of the following:

≥45 years of age and has not had menses for >2 years.

Amenorrheic for >2 years without a hysterectomy and oophorectomy and a follicle-stimulating hormone value in the postmenopausal range upon pre-study (screening) evaluation.

Post hysterectomy with oophorectomy. Documented hysterectomy or oophorectomy must be confirmed with medical records of the actual procedure or confirmed by an ultrasound. In case of oophorectomy alone, hormone level assessment (follicle-stimulating hormone, estradiol) will be done locally at Screening to confirm postmenopausal status.

Patients who are on ovarian function suppression also qualify.

10. Patient agrees to, and is willing and able to arrive at the hospital/clinic in a fasted state (>8 hours) on designated fasting days.

11. Patient has adequate bone marrow and organ function as defined by the following laboratory values (as assessed by central laboratory for eligibility):
Platelet count≥140×109/L

In absence of liver metastases, alanine aminotransferase (ALT) and aspartate aminotransferase (AST)<2.5×the upper limit of normal (ULN). If the patient has liver metastases, ALT and AST<5×ULN.

Total bilirubin≤1.5×ULN except for patient with Gilbert's syndrome who may only be included if the total bilirubin is ≤3.0×ULN or direct bilirubin≤1.5×ULN.
Fasting serum amylase≤2×ULN
Fasting serum lipase≤ULN
Hemoglobin≥9 g/dl
Absolute neutrophil count [ANC])≥1500/mL
Creatinine clearance>60 mL/min using Cockcroft-Gault equation
Albumin>3.5 gm/dL

### Example 2—Phase 2 Study of Compound 1 Combination with Eribulin Mesylate for Patients with Metastatic Triple-Negative Breast Cancer and Metabolic Dysfunction

The following non-limiting example is a description of a Phase 2 study to determine the anti-tumor effects of a combination of Compound 1 and eribulin mesylate in certain triple-negative breast cancer populations.

Overview: This example describes a placebo-controlled phase 2 randomized control trial to test the use of Compound 1 in combination with eribulin mesylate for the treatment of histologically-confirmed hormone receptor negative, HER2-negative (i.e. triple-negative) metastatic breast cancer and concomitant metabolic dysfunction. The trial will include a safety run-in period, during which time 15 patients will receive Compound 1 at 49 mg/m² administered every 2 weeks in combination with eribulin mesylate administered on days 1 and 8 of a 21-day cycle. After safety confirmation, an additional 40 patients will be randomized (2:1) to receive Compound 1 in combination with eribulin mesylate versus placebo plus eribulin mesylate. The primary objective of this study is to determine preliminary biologic efficacy. This outcome will be assessed by change in insulin resistance. Secondary objectives of the study include, but are not limited to objective response rate (ORR), progression free survival (PFS), duration of response, safety and tolerability, patient reported outcomes, and changes in metabolic markers and other biomarkers.

Patient Population: Men and women with histologically-confirmed, triple-negative metastatic breast cancer (MBC), who have received up to 2 prior lines of treatment for metastatic disease, and who have baseline metabolic dysfunction (baseline hemoglobin A1c>5.5 and/or body mass index (BMI)≤30 kg/m²).

Design: First, a safety run-in period will be performed in which the first 15 patients enrolled will be assigned to receive the study drug Compound 1 in combination with eribulin mesylate. Once safety is confirmed, the next 40 patients who are enrolled will be randomized (2:1) to receive Compound 1 at the previously identified MTD of 49 mg/m² every 2 weeks in combination with standard dose eribulin mesylate (investigational arm) versus placebo plus eribulin mesylate chemotherapy (control arm). The primary objective of this study is to determine preliminary biologic efficacy. This outcome will be assessed by change in insulin resistance. Secondary objectives of the study include, but are not limited to objective response rate (ORR), progression free survival (PFS), duration of response, safety and tolerability, patient reported outcomes, and changes in metabolic markers and other biomarkers.

Treatment Plan: Patients in both the investigational arm and control arm will receive standard dose eribulin mesylate 1.4 mg/m² on days 1 and 8 of an every 21 day cycle. Patients will receive Compound 1 starting at 49 mg/m² or placebo 1 week prior to starting eribulin mesylate and then on a Q14D basis in combination with eribulin mesylate. Treatment with the combination of Compound 1 and eribulin mesylate will continue until the development of progressive disease (PD) or another criterion for withdrawal is met. The dosage of Compound 1 can be increased to 65 mg/m² or decreased to 36 mg/m² depending on PK and safety analysis.

### Eligibility Criteria:

Male or female
Histologically and/or cytologically confirmed diagnosis of triple-negative metastatic breast cancer defined as estrogen and progesterone receptor staining<10%; and HER2-negative defined as IHC 0 to 1+ (note: if IHC is equivocal, non-amplified status by FISH is acceptable)
Advanced (local regionally recurrent, not amenable to curative therapy or surgery) or metastatic stage with up to 2 prior lines of therapy in the advanced or metastatic setting
Evidence of metabolic dysfunction defined as HbA1c>5.5 and/or BMI≥30 kg/m²
Measurable disease per the Response Evaluation Criteria in Solid Tumors, version 1.1 (RECIST 1.1), OR at least one evaluable, predominantly lytic bone lesion.
Eastern Cooperative Oncology Group Performance Status (ECOG-PS)≤1.
Adult ≤18 at the time of informed consent and has provided written informed consent before the performance of any study-related activities and according to local guidelines.
Adequate bone marrow and organ function as defined by the following laboratory values (as assessed by central laboratory for eligibility):
   Absolute neutrophil count (ANC)≤1,000 μL
   Platelet count≥140,000 μL
   Hemoglobin≥9.0 g/dL:
   Calcium (corrected for serum albumin) and magnesium≤Grade 1 according to National Cancer Institute (NCI) Common Terminology Criteria for Adverse Events (CTCAE), version 5.0, and not considered by the Investigator to be clinically significant.
   Potassium within normal limits, with or without correction with supplements.
   In absence of liver metastases, alanine aminotransferase (ALT) and aspartate aminotransferase (AST)≤2.5×the upper limit of normal (ULN). If the patient has liver metastases, ALT and AST≤5×ULN.
   Total bilirubin≤1.5×ULN except for patient with Gilbert's syndrome who may only be included if the total bilirubin is ≤3.0×ULN or direct bilirubin≤1.5×ULN.
   Creatinine≤1.5 mg/dL.
Patient is willing and able to comply with the study requirements, including the ability to fast prior to treatment days.
If sexually active female of childbearing potential, willing to use barrier contraception
If a sexually active male, willing to use barrier contraception

Primary Objective: The primary endpoint of the study is to determine the biologic efficacy of Compound 1 in combination with eribulin mesylate, wherein the efficacy is defined by change in insulin resistance scores (HOMA-IR) on or after cycle 4 of treatment (i.e. ˜12 weeks). Such assessment will be performed within 2 weeks after cycle 4 day 1. Study participants who receive at least 3 treatment cycles will be considered evaluable for the primary endpoint. The Homeostasis Model Assessment of Insulin Resistance (HOMA-IR) will be used to assess insulin resistance. HOMA-IR is calculated as follows: fasting serum insulin (μU/mL)×fasting plasma glucose (mmol L−1)/22.5).

### Secondary Objectives:

Safety and tolerability: Type, frequency and severity of treatment-emergent adverse events (TEAEs) and laboratory toxicities per the NCI CTCAE version 5.0.

3 and 6 month ORR: Response rate will be assessed by RECIST v1.1 as follows:
Partial response (PR) is at least a 30% reduction in the sum of diameters of target lesions, taking as reference the baseline sum diameters.
Complete response (CR) is the disappearance of all target lesions. Any pathological lymph nodes (whether target or non-target) must have reduction in short axis to <10 mm.
Stable disease (SD) is neither sufficient shrinkage to qualify for PR nor sufficient increase to qualify for PD, taking as reference the smallest sum diameters while on study.
Progressive disease (PD) requires a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).
Duration of response, defined as the time from the first occurrence of a documented objective tumor response to the time of radiographic progression (per investigator using RECIST v1.1) or death from any cause on study, whichever occurs first

3 month, 6 month, and median PFS: PFS will be assessed using RECIST v1.1 and is defined as time from treatment assignment to disease progression or death, whichever comes first.

Changes in circulating metabolic and other biomarkers: Fasting blood will be collected at baseline and every cycle. Circulating levels of glucose, insulin, HbA1C, leptin, adiponectin, Apelin, FGF21, and lipids will be measured in clinical chemistry laboratories. Other exploratory biomarkers, including angiogenic markers bFGF, VEGF (A, C and D), inflammatory markers IL-5, IL-6, hs-CRP, GM-CSF, Resistin, metastatic markers sFRP-1, sFRP-5, MetAP2 marker Cyclophilin A, as well as SHBG, and circulating tumor DNA (ctDNA) will be measured in clinical chemistry laboratories.

Changes in body composition: Body composition will be assessed using computerized tomography (CT). The volume of fat and muscle at the level of the 3rd lumbar vertebra will be analyzed using commercially available software (iNtuition, TeraRecon) using the CT scans that are to be performed to evaluate tumor burden (baselin, q3 months). CT-based lumbar muscle volume (30 mm³ with a superior level of L3) will be measured by two readers. The volumetric slabs will be analyzed for the presence of skeletal muscle using a semi-automated technique. First, attenuation thresholds of −29 and 150 HU will be applied to the entire image volume. A color-coded map of voxels with mean attenuation values within the range of −29 and 150 HU will be generated. The non-muscular soft tissues (abdominopelvic viscera, large blood vessels, spinal cord, and portions of the bone marrow) will be manually excluded by drawing a region of interest around the identified tissue region. Organs containing smooth muscle will be excluded due to the variation in muscle volume during the peristaltic activity of the gut. A series of images containing only skeletal muscle will be generated, which yields a volumetric calculation of whole-body skeletal muscle. Subcutaneous and visceral adipose tissue volumes will be segmented from the same L3 volumetric slab using a similar semi-automated approach with iNtuition.

Quality of Life: QOL will be assessed by the Functional Assessment of Cancer Therapy-Breast (FACT-B) (version 4) scale. As would be appreciated by the skilled artisan, the FACT-B contains 37-items, divided into four primary subscales for physical well-being (7 items), functional well-being (7 items), emotional well-being (6 items), and social/family well-being (7 items) that comprise the FACT-General (FACT-G) scale, in addition to a breast cancer subscale (10 items). The five subscales will be summed to obtain the FACT-B score (for a total score of 148). A FACT-G score (27 items, excluding the breast cancer subscale; total score of 108) will also be computed. All items are rated on a 0 to 4 Likert scale, using the following response format: 0=not at all; 1=a little bit; 2=somewhat; 3=quite a bit; 4=very much. Respondents will be asked to respond to each question as it applies to the past 7 days. Higher scores on the FACT-B indicate higher QOL.

Plasma concentration-time (PK): The PK profile of Compound 1 and its primary metabolite and other metabolites when given in combination with eribulin mesylate will be collected in the investigational arm as follows: For the first 15 patients, samples are to collected at 3 hours (10 min) post-Compound 1 on C1D1; 24 hours post Compound 1 at C1D2; 48 hours post Compound 1 at C1D3; pre-Compound 1 at C1D8; pre and 3 hours post-Compound 1 at C1D15; pre-Compound 1 at C2D1; and pre Compound 1 at C2D8. For the remaining 40 patients, PK samples will be collected at 3 hours (10 min) post Compound 1 on C1D1; pre-Compound 1 on C1D8; pre- and 3 hours post Compound 1 on C1D15; pre-Compound 1 on C2D1; and pre-Compound 1 on C2D8.

### Example 3—Treatment of Breast Cancer Using a Combination of Compound 1 and Eribulin Mesylate

The following is a non-limiting example demonstrating the treatment of breast cancer in a mouse model using a combination of Compound 1 and Eribulin.

Animals: 110 Female C57BL/6J mice were received from Jackson Labs and housed in groups of 3/cage. Beginning at six (6) weeks of age, mice were fed a high-fat, high-sucrose diet (D12451) for 16 weeks, prior to tumor implantation. Mice were maintained on this diet for the duration of the study.

Cell Culture: the E0771 cell line was obtained from CH3 Biosystems, LLC, and cultured DMEM with 10% FBS. As would be appreciated by the skilled artisan, E0771 cells are a model of triple-negative breast cancer. On the day of implantation, the cells were washed with phosphate buffered saline (PBS) and harvested via trypsinization (incubation with 0.25% Trypsin for 5 min at 37° C.), the trypsin is deactivated with twice the volume of complete media. The harvested cells were pelleted by centrifugation at a rate of 2500 rpm for 5 minutes at 18° C. Once the cells were pelleted the supernatant was removed and the pellet resuspended in 30 mL's of PBS. Following cell and viability count, the cells were pelleted for 5 minutes at 2500 rpm at 18° C. and resuspended in 50% PBS and 50% Matrigel at a final concentration of 1×10⁶ per mL (1×10⁵/mouse) for implantation.

Tumor Cell Implantation: the E0771 cells were implanted into the fourth mammary gland in a mixture of PBS and Matrigel at a volume of 100 μl per mouse. Mice were anesthetized with 4% Isoflurane and 1.2% Oxygen, while anesthetized the cells were injected in to the lower right mammary fat pad.

Tumor Assessment: beginning 7 day's post cell implantation all animals were weighed, marked and then tumors measured twice weekly (length and width) using a wireless Mitutoyo UWAVE-T digital calipers in conjunction with UWAVE-R to record the measurements. The tumor volume is calculated

$$( ( l * {w}^{2} ) * \frac{π}{6} )$$

using Microsoft Office Excel software. Once the average tumor volume reached approximately 100 mm³ the mice were randomized and placed into treatment groups of 6 animals per group (based on tumor volume). Actual mean tumor volume was 118 mm³/group).

Treatment: nine groups of mice were treated as put forth in Table A. Body weights were recorded twice weekly beginning on day 1 of the study, tumor measurements were taken the same day as body weights for the duration of the study. The mice in group 1 were dosed intraperitoneally (IP) with Vehicle 1 (5% EtOH, 95% Water) Q4D and subcutaneously (SC) with vehicle (5% mannitol in water) Q4D for a total of 7 doses with each vehicle. The mice administered eribulin mesylate were dosed intra-peritoneally Q4D with a variable number of doses due to poor toleration of the 2 mg/kg dose. The mice administered Compound at 6 mg/kg and 12 mg/kg were dosed subcutaneously (SC) Q4D fora total of 7 doses. All IP and SC dose volumes were calculated based on 10 mL/kB of body weight.

TABLE A

| | | Number | | Dose | | |
|---|---|---|---|---|---|---|
| | | of mice | Dose | Route/ | | |
| Group | Group | per | (mg/ | Fre- | # of | Pro- |
| # | ID | group | kg) | quency | Doses | cedures |
| 1 | Vehicle | 9 | NA | SC/Q4D | 7 | Body |
| | | | | IP/Q4D | | weight |
| 2 | Compound | 9 | 6 | SC/Q4D | 7 | and tumor |
| | 1 | | | | | volume - |
| 3 | Compound | 9 | 12 | SC/Q4D | 7 | 2x/week |
| | 1 | | | | | (BIW) |
| 4 | Eribulin | 9 | 1 | IP/Q4D | 7 | |
| | mesylate | | | | | |
| 5 | Eribulin | 9 | 2 | IP/Q4D | 5 | |
| | mesylate | | | | | |
| 6 | Compound | 9 | 6 | SC/Q4D | 7 | |
| | 1 + | | 1 | IP/Q4D | 6 | |
| | Eribulin | | | | | |
| | mesylate | | | | | |
| 7 | Compound | 9 | 12 | SC/Q4D | 7 | |
| | 1 + | | 1 | IP/Q4D | 6 | |
| | Eribulin | | | | | |
| | mesylate | | | | | |
| 8 | Compound | 9 | 6 | SC/Q4D | 7 | |
| | 1 + | | 2 | IP/Q4D | 4 | |
| | Eribulin | | | | | |
| | mesylate | | | | | |
| 9 | Compound | 9 | 12 | SC/Q4D | 7 | |
| | 1 + | | 2 | IP/Q4D | 4 | |
| | Eribulin | | | | | |
| | mesylate | | | | | |

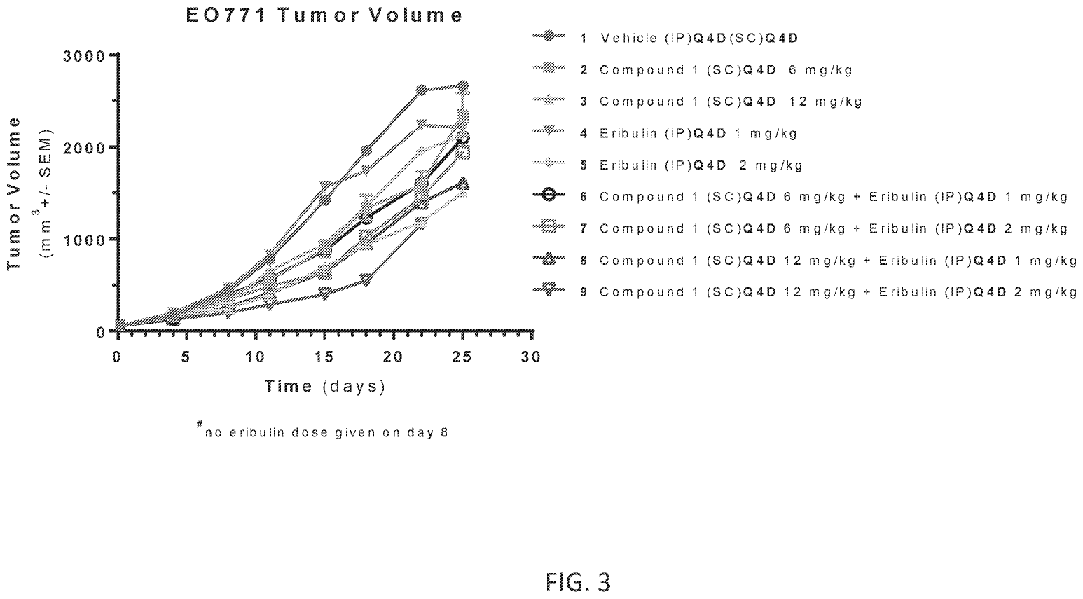

FIG. 3 is a graph of tumor volume over the course of the study in the different treatment groups. In treatment groups 5, 7 and 9, no eribulin mesylate dose was administered on day 8.

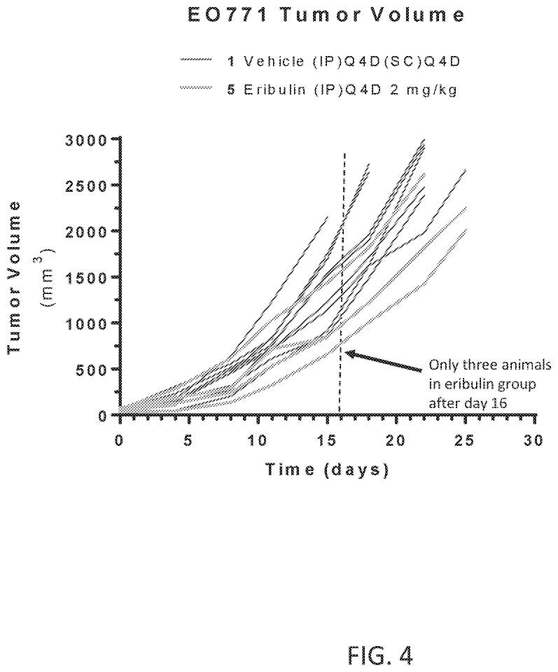

FIG. 4 is a graph of tumor volume in individual animals treated with either vehicle control or eribulin mesylate at 2/mg/kg. As shown in FIG. 4, eribulin mesylate at 2 mg/kg, IP/Q4D was not well tolerated, leading to multiple early terminations. Accordingly, dosing of eribulin mesylate was held on day 8 and resumed on day 11, but on a Q7D schedule. This change was also implemented in treatment groups 8 and 9.

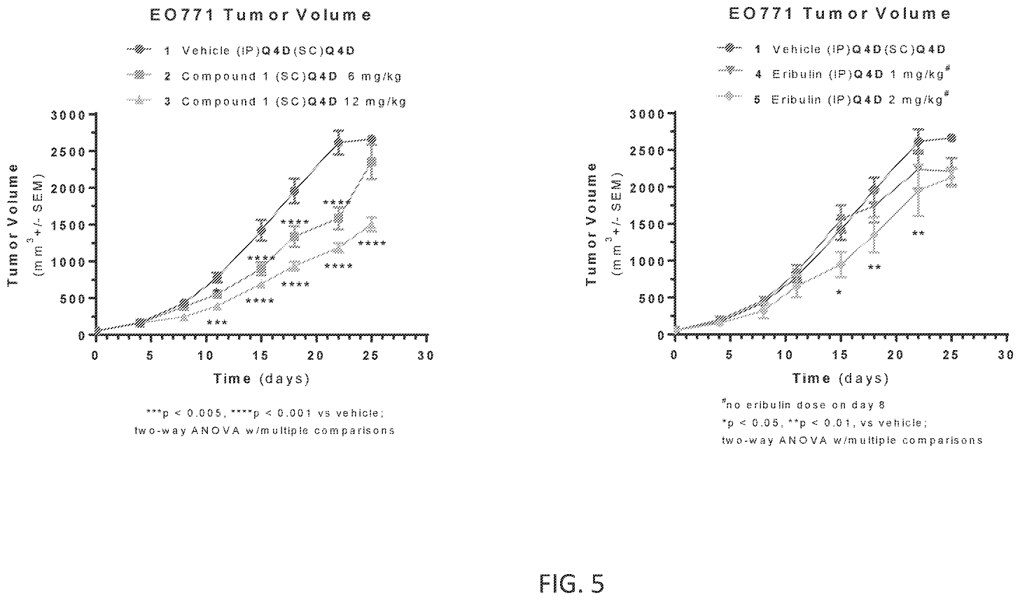

FIG. 5 is a series of graphs showing tumor volume in treatment groups 1-5 over the course of the study.

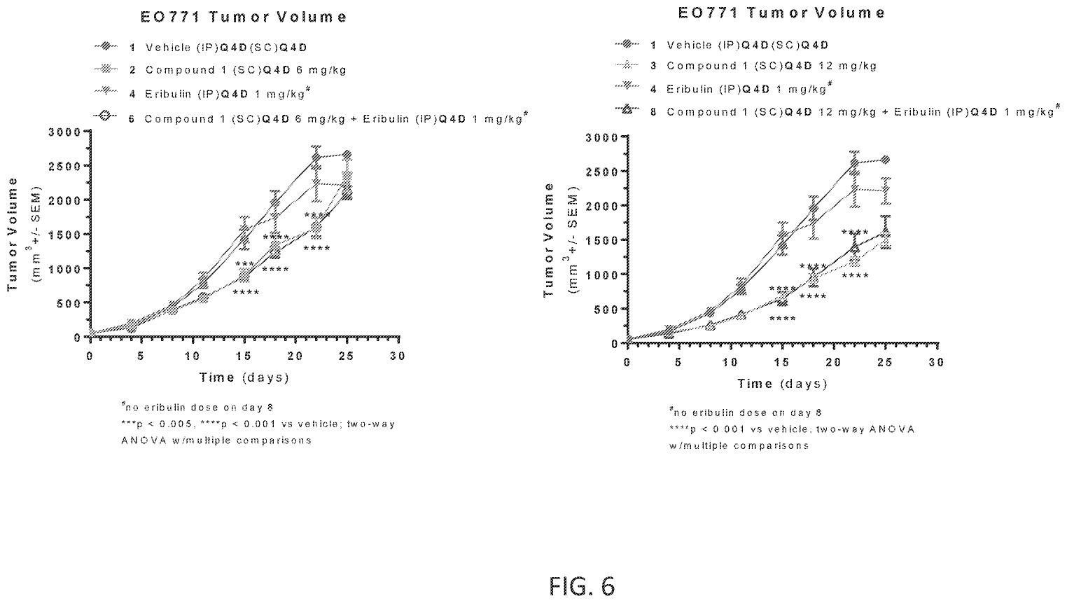

FIG. 6 is a series of graphs showing tumor volume in treatment groups 1, 2-4, 6 and 8 over the course of the study.

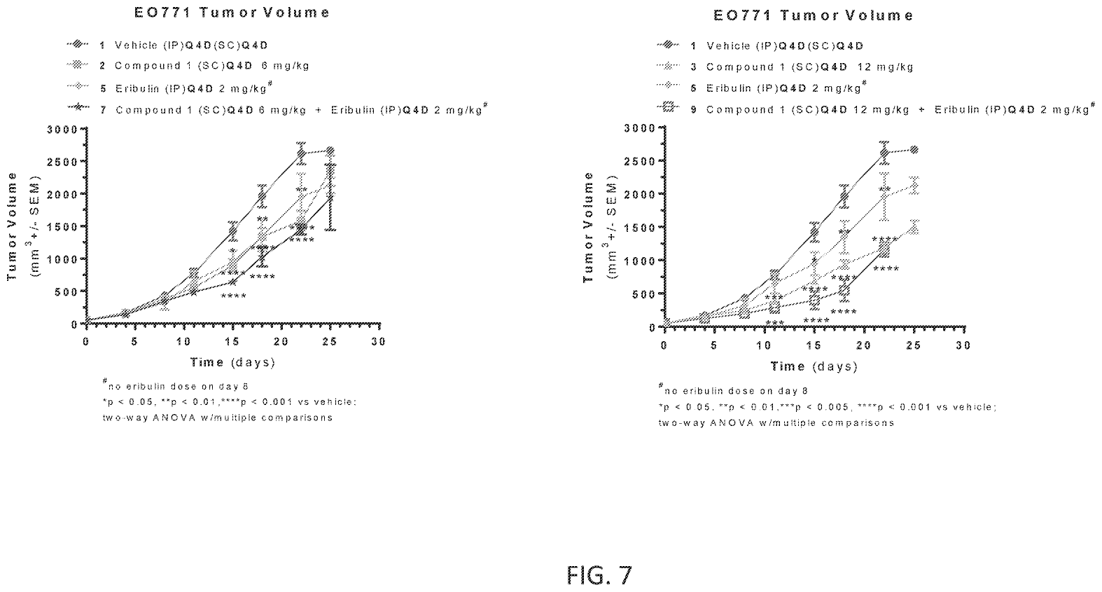

FIG. 7 is a series of graphs showing tumor volume in treatment groups 1-3, 5, 7 and 9 over the course of the study.

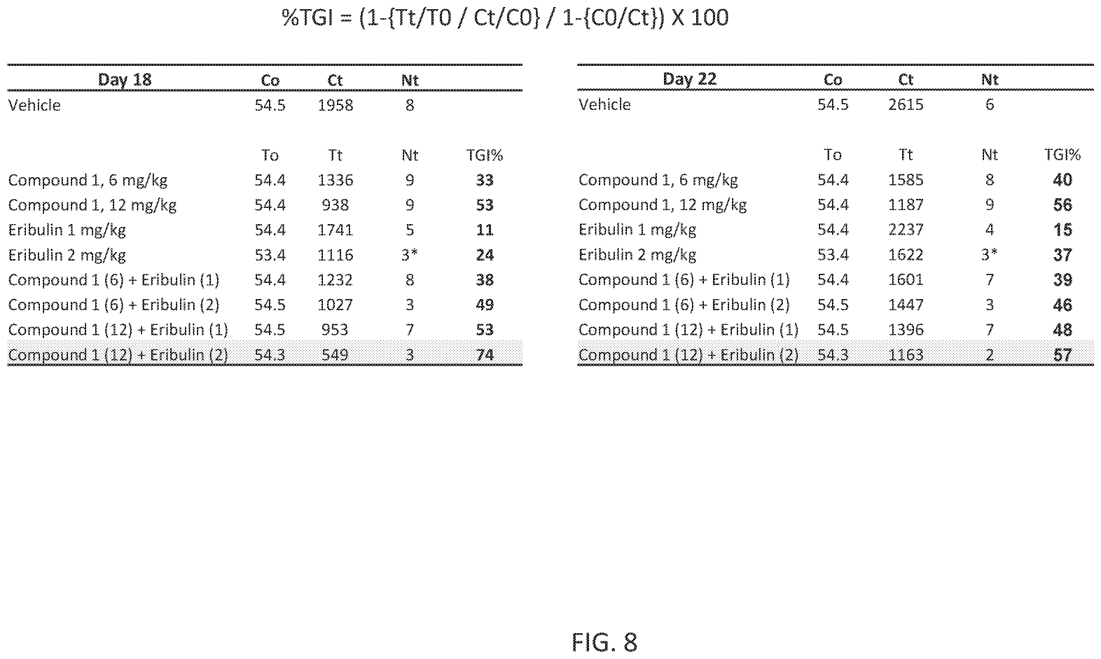

FIG. 8 is a series of charts showing tumor growth inhibition in the various treatment groups on Day 18 and Day 22 of the study.

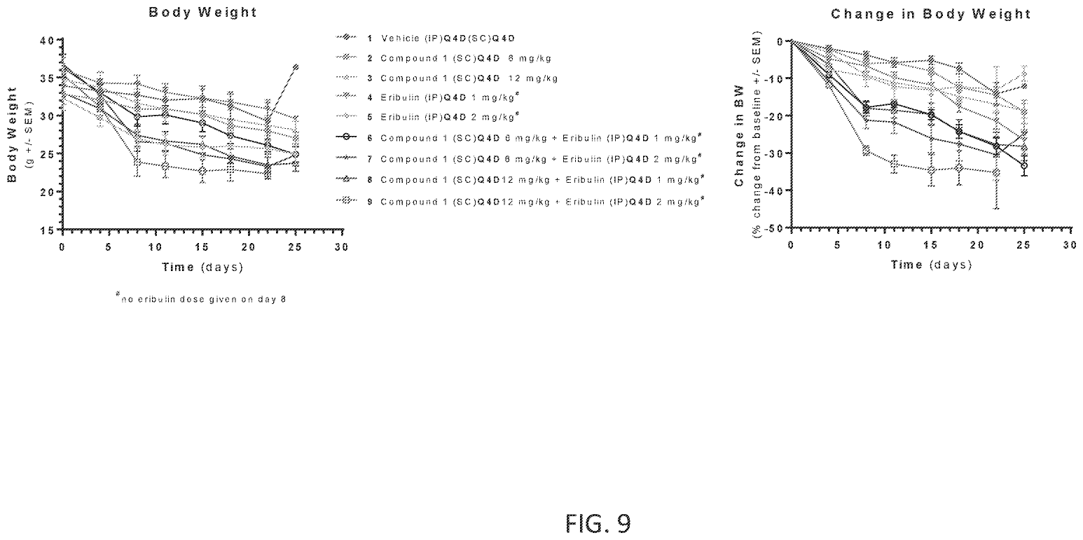

FIG. 9 is a series of graphs showing body weight and change in body weight in the various treatment groups over the course of the study. As shown in FIG. 9, all groups, including the vehicle group lost weight. Weight loss in the Compound 1 and eribulin mesylate groups (groups 2-5) was increased as compared to the vehicle group. Weight loss in the combination groups was consistently greater than the vehicle group.

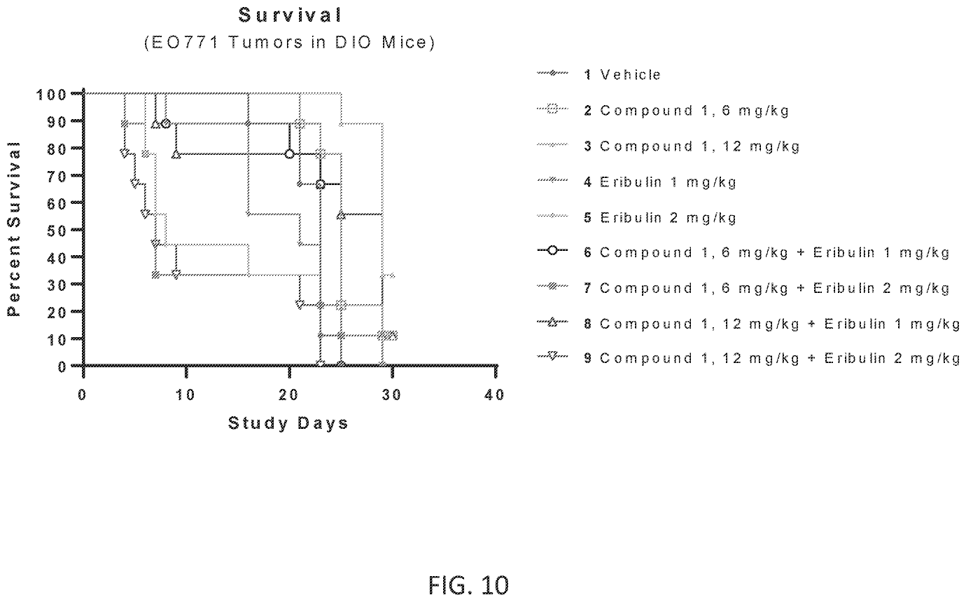

FIG. 10 is a graph showing percentage survival in the various treatment groups over the course of the study. As shown in FIG. 10, the combination of Compound 1 at 12/mg/kg and eribulin mesylate at 1 mg/kg resulted in increased survival relative to the vehicle group.

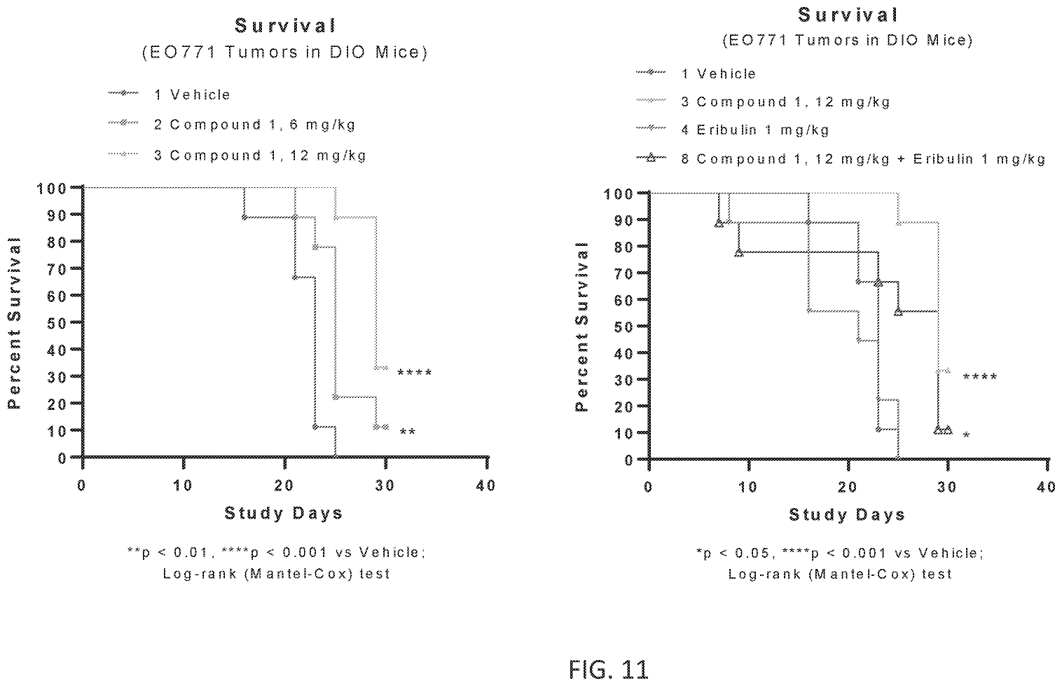

FIG. 11 is a series of graphs showing percentage survival in treatment groups 1-4 and 8 over the course of the study.

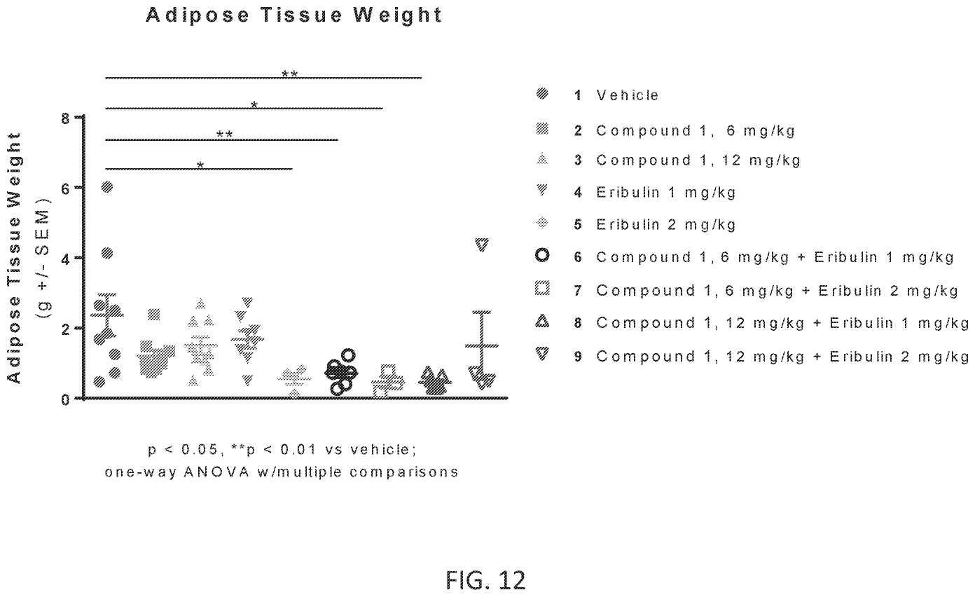

FIG. 12 is a graph showing the adipose tissue mass measured in the various treatment groups. As shown in FIG. 12, reductions in adipose tissue mass were observed in the treatment groups 5-8.

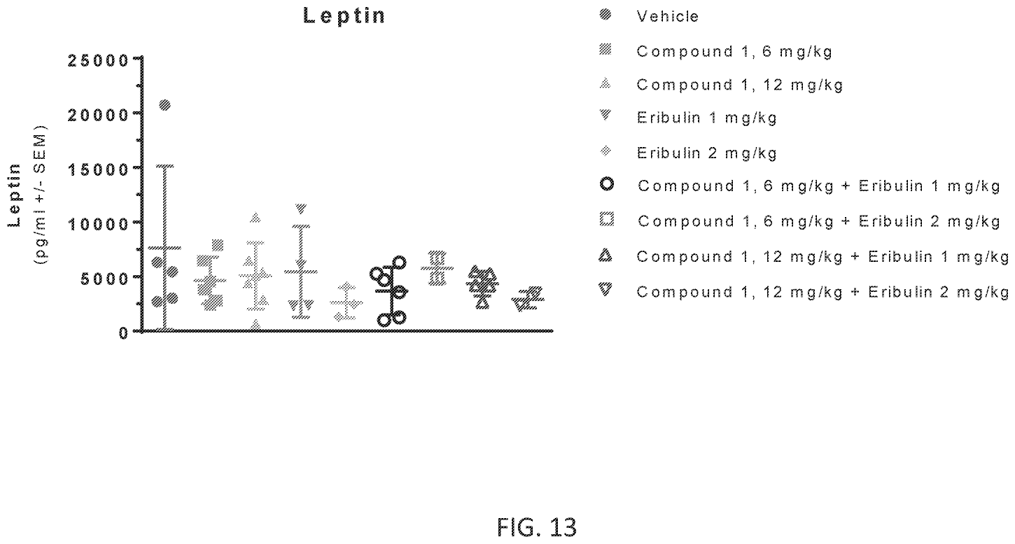

FIG. 13 is a graph showing leptin levels in the various treatment groups.

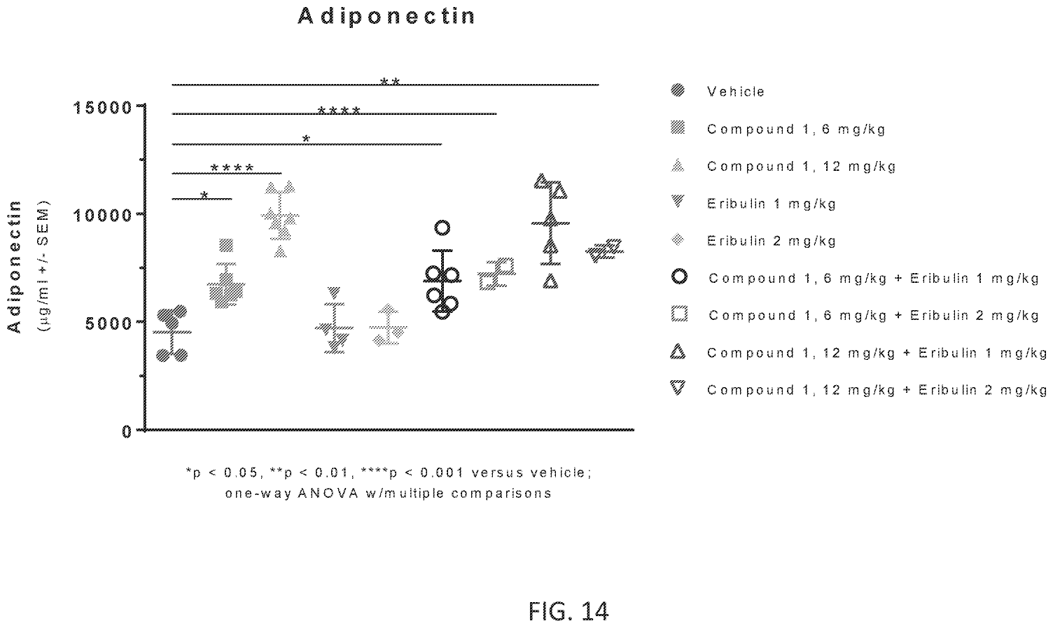

FIG. 14 is a graph showing plasma adiponectin levels in the various treatment groups.

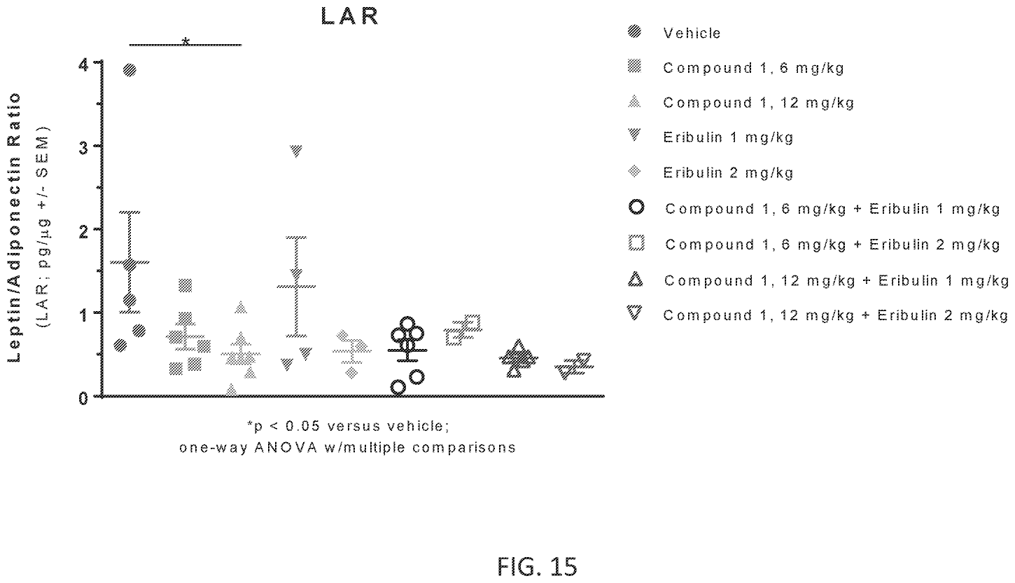

FIG. 15 is a graph showing the leptin/adiponectin ration (LAR) in the plasma samples isolated from the various treatment groups.

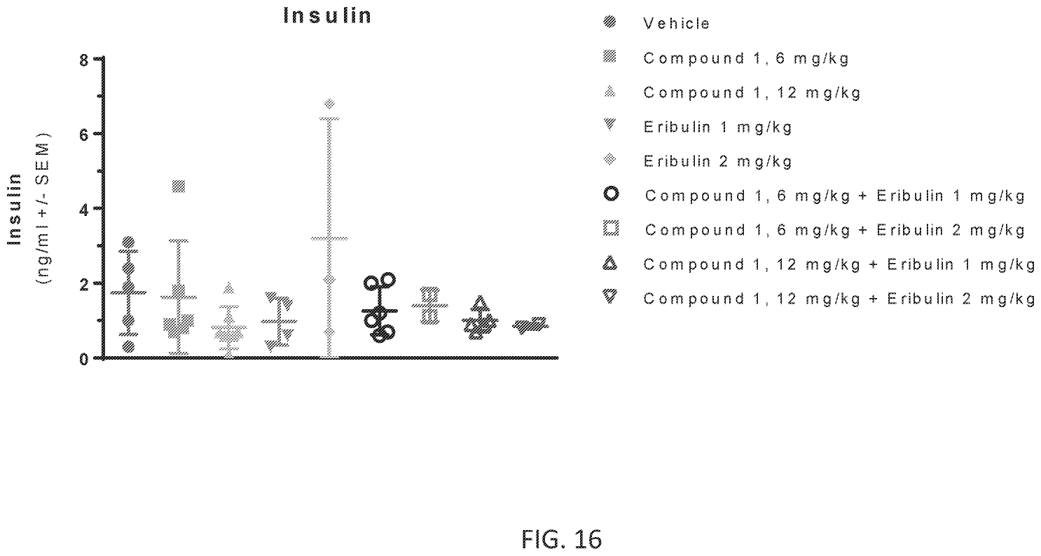

FIG. 16 is a graph showing plasma insulin levels in the various treatment groups.

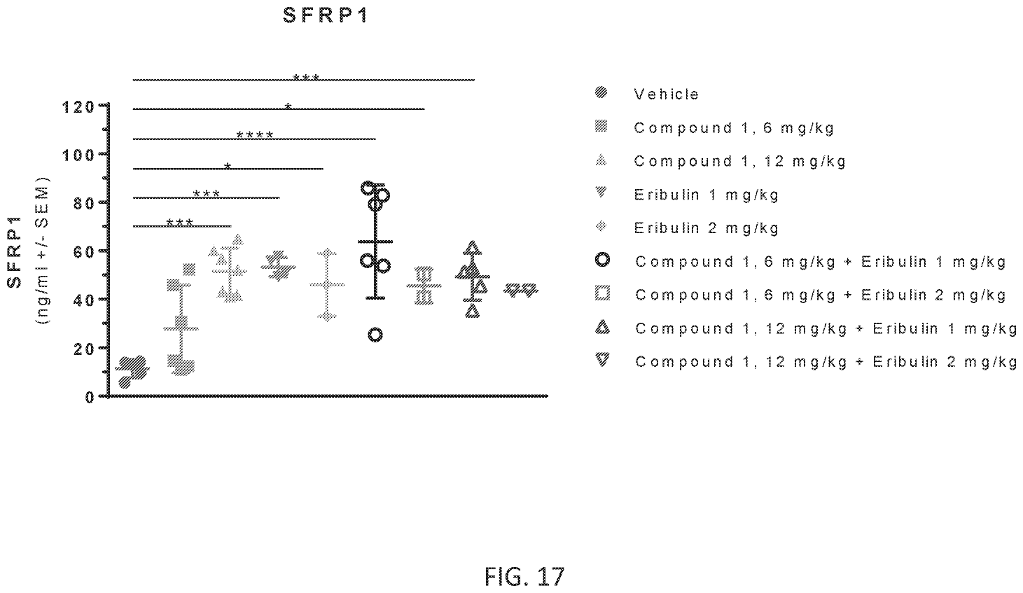

FIG. 17 is a graph showing plasma SFRP1 levels in the various treatment groups.

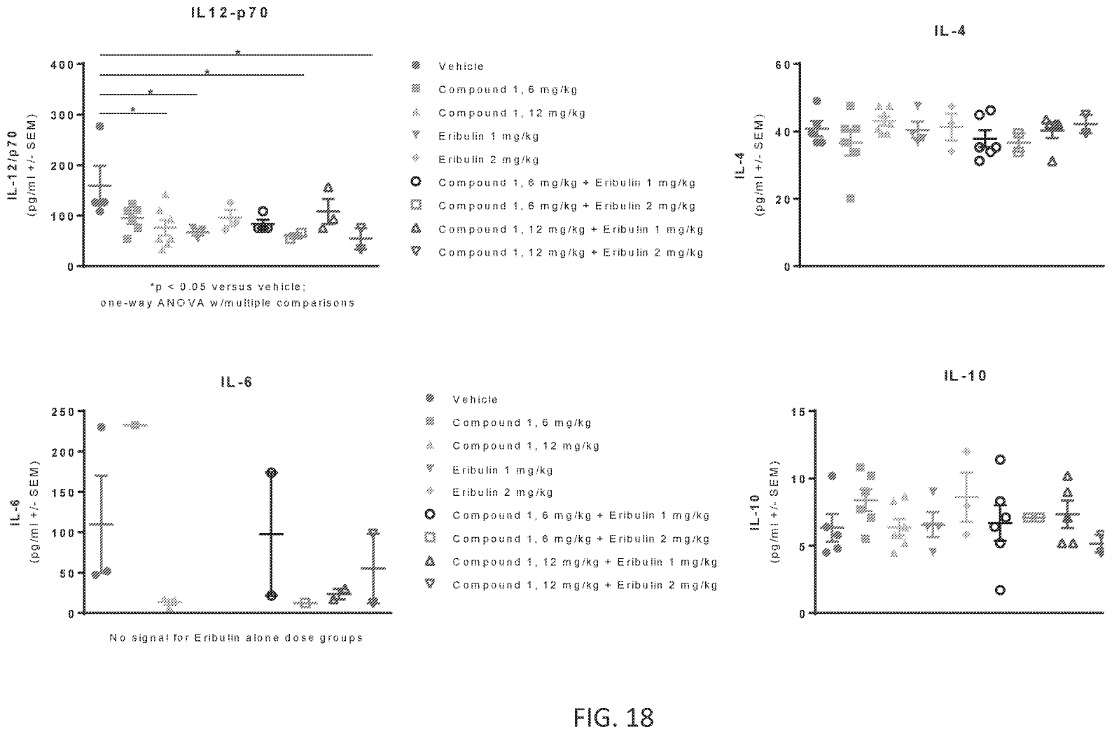

FIG. 18 is a series of graphs showing plasma interleukin levels in the various treatment groups.

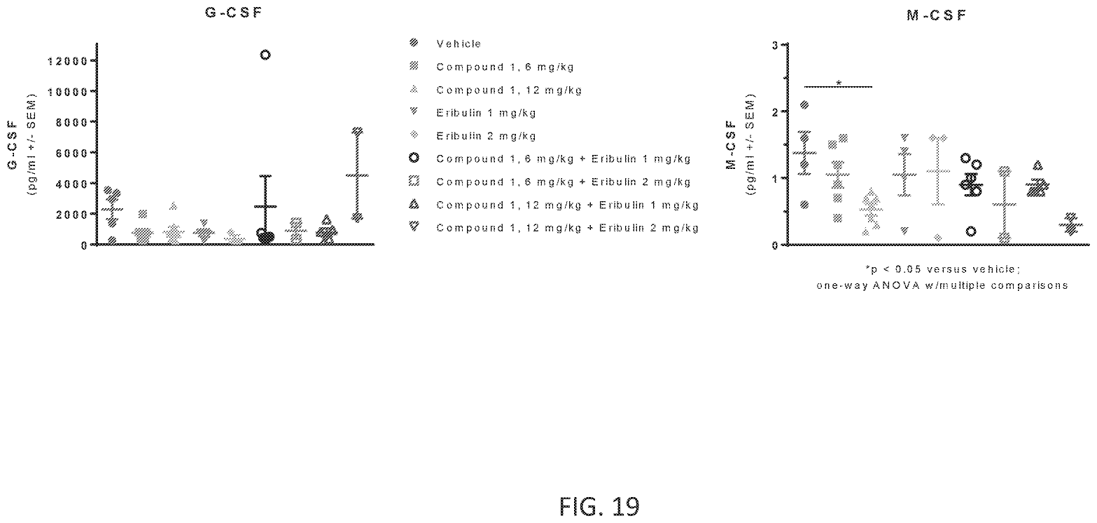

FIG. 19 is a series of graphs showing plasma hematopoietic growth factor levels (G-CSF and M-CSF) in the plasma samples isolated from the various treatment groups.

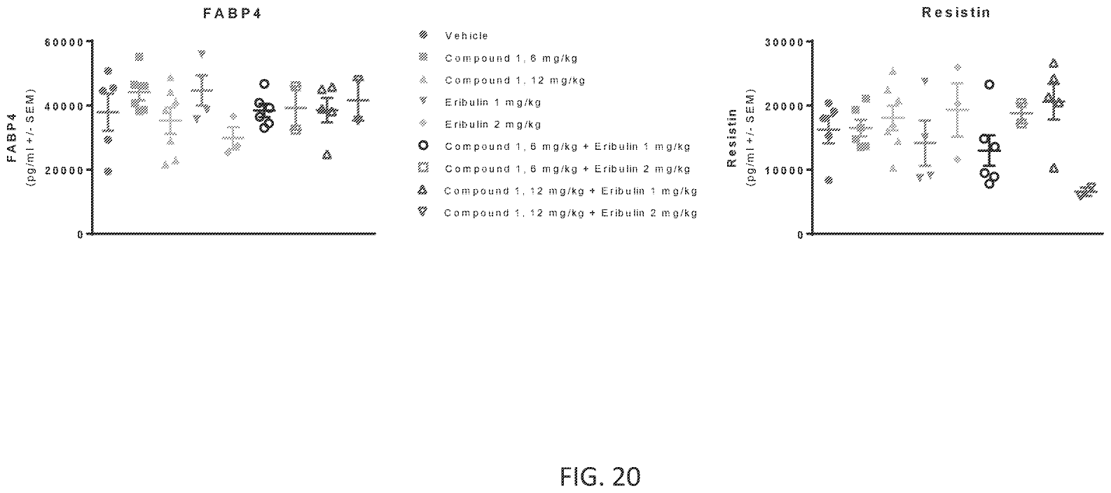

FIG. 20 is a series of graphs showing the levels of FABP4 and Resistin in plasma samples isolated from the various treatment groups.

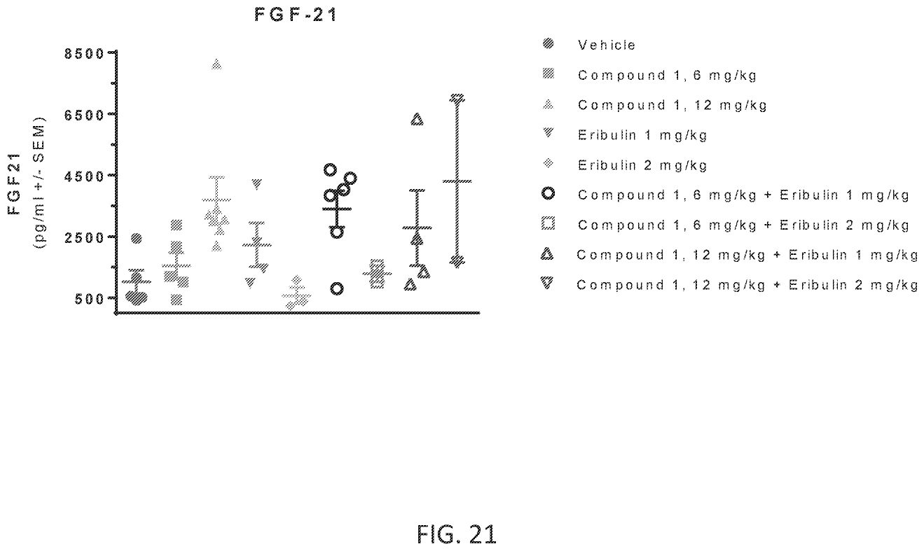

FIG. 21 is a graph showing plasma FGF-21 levels in the various treatment groups.

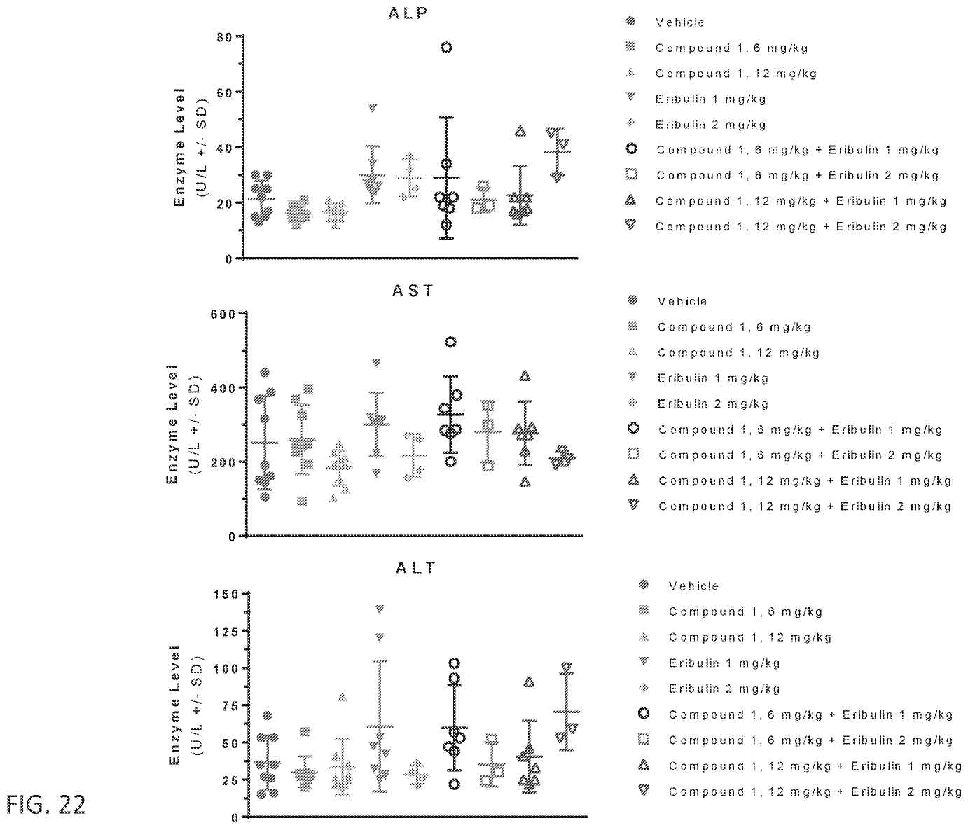

FIG. 22 is a series of graphs showing ALP, ALT and AST levels in the various treatment groups.

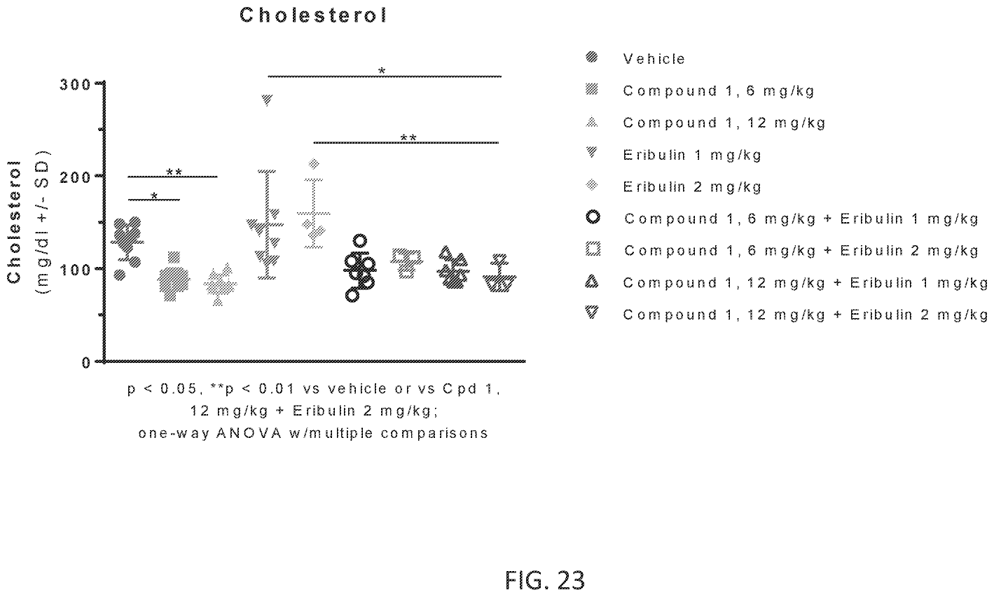

FIG. 23 is a graph showing cholesterol levels in the various treatment groups.

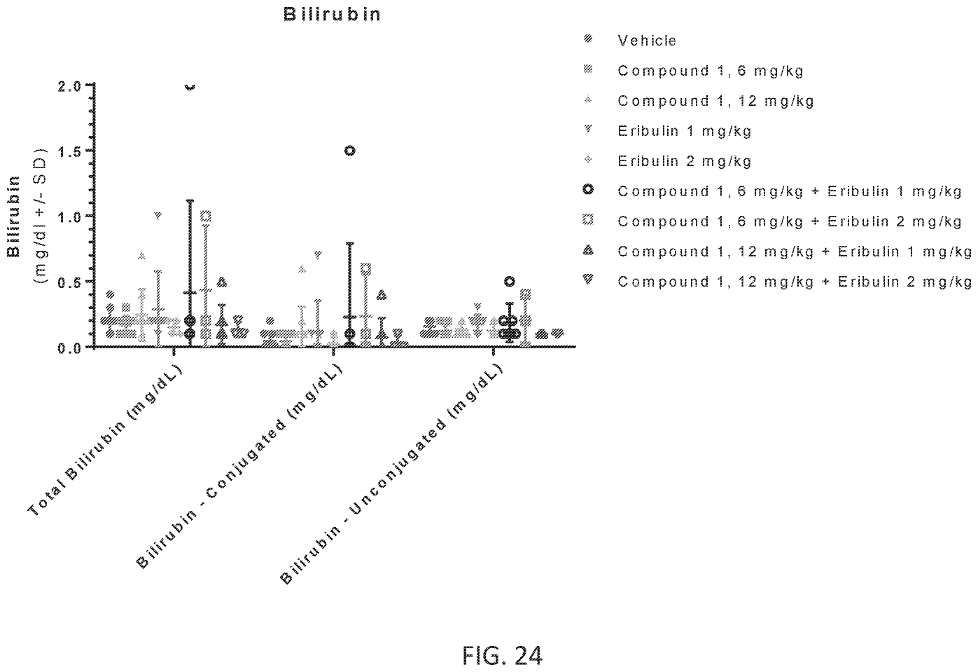

FIG. 24 is a graph showing bilirubin levels in the various treatment groups.

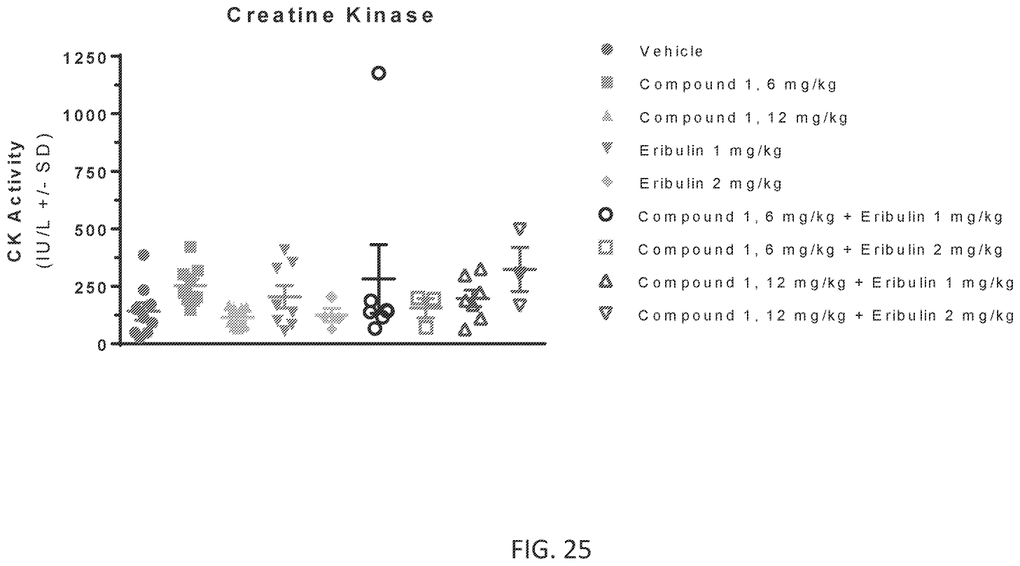

FIG. 25 is a graph showing creatine kinase levels in the various treatment groups.

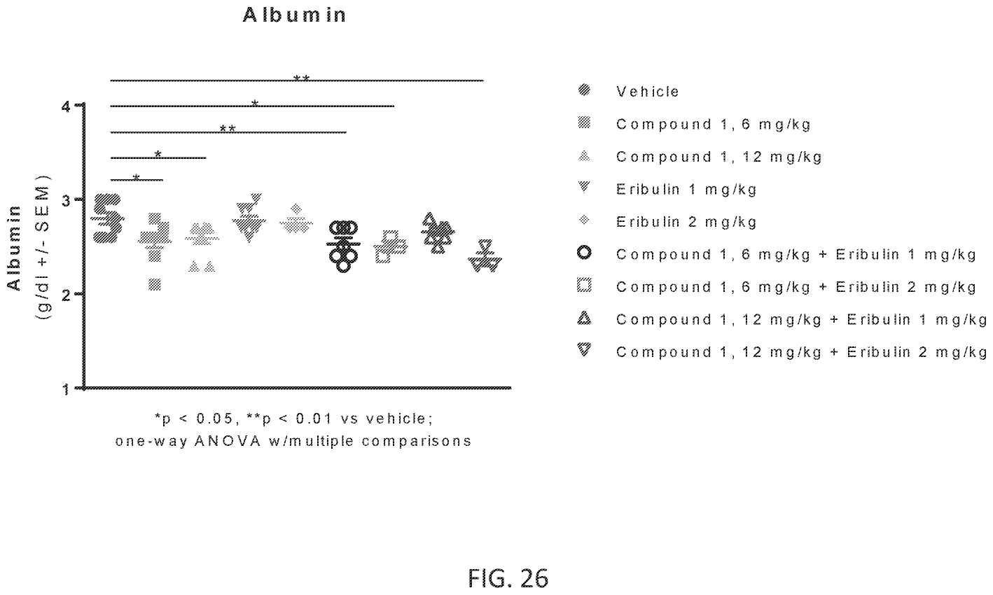

FIG. 26 is a graph showing albumin levels in the various treatment groups.

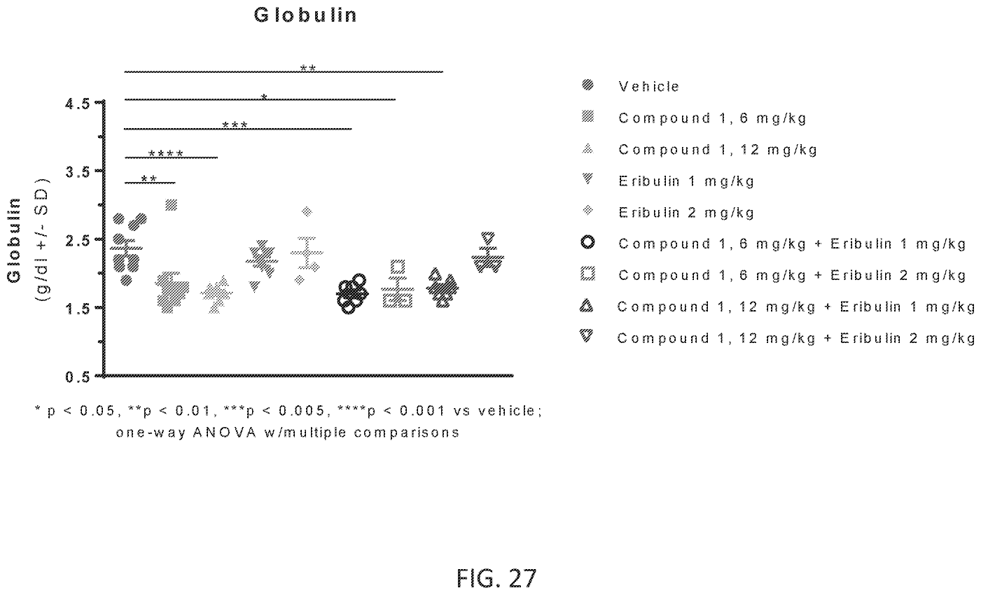

FIG. 27 is a graph showing globulin levels in the various treatment groups.

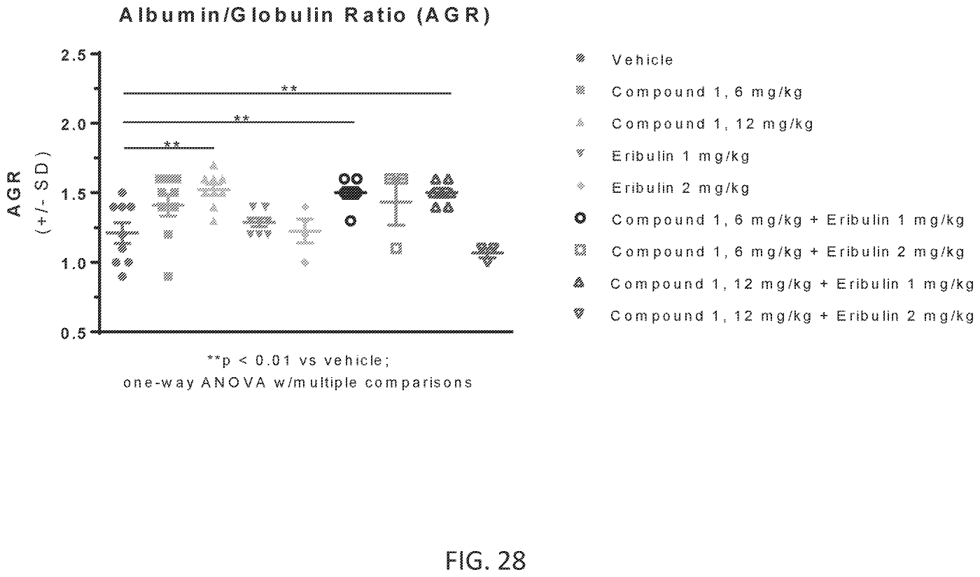

FIG. 28 is a graph showing albumin to globulin ratio (AGR) in the various treatment groups.

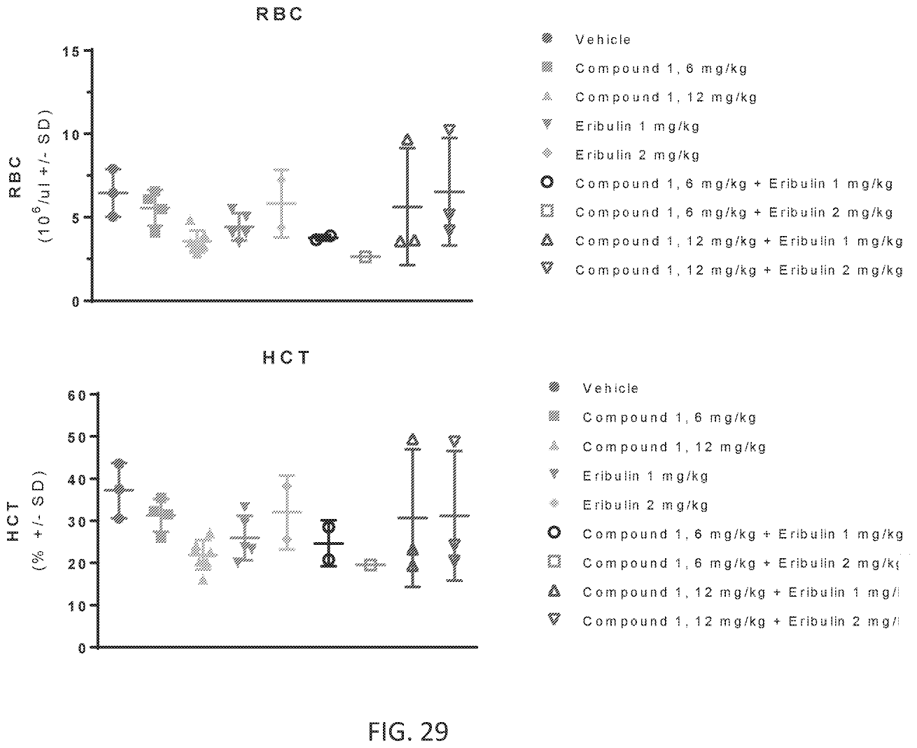

FIG. 29 is a series of graphs showing red blood cell (RBC) and hematocrit (HCT) levels in the various treatment groups.

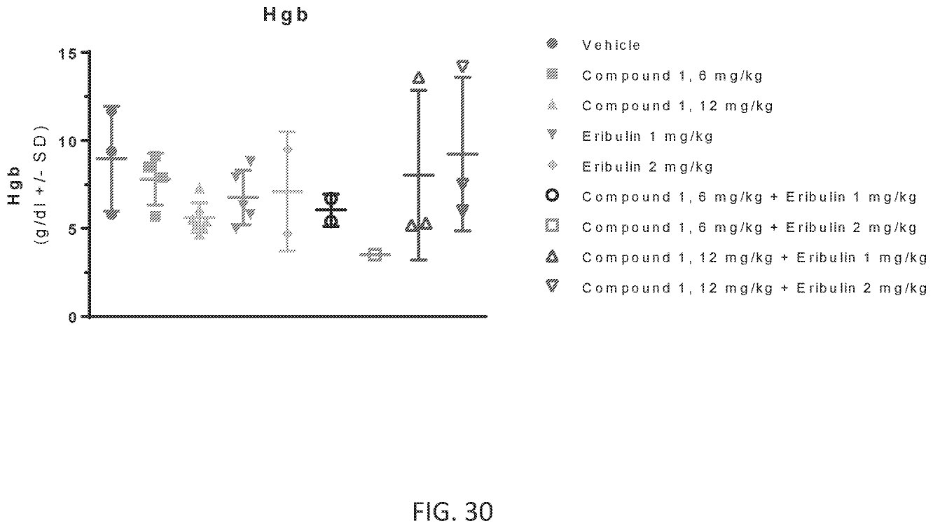

FIG. 30 is a graph showing hemoglobin (Hgb) levels in the various treatment groups.

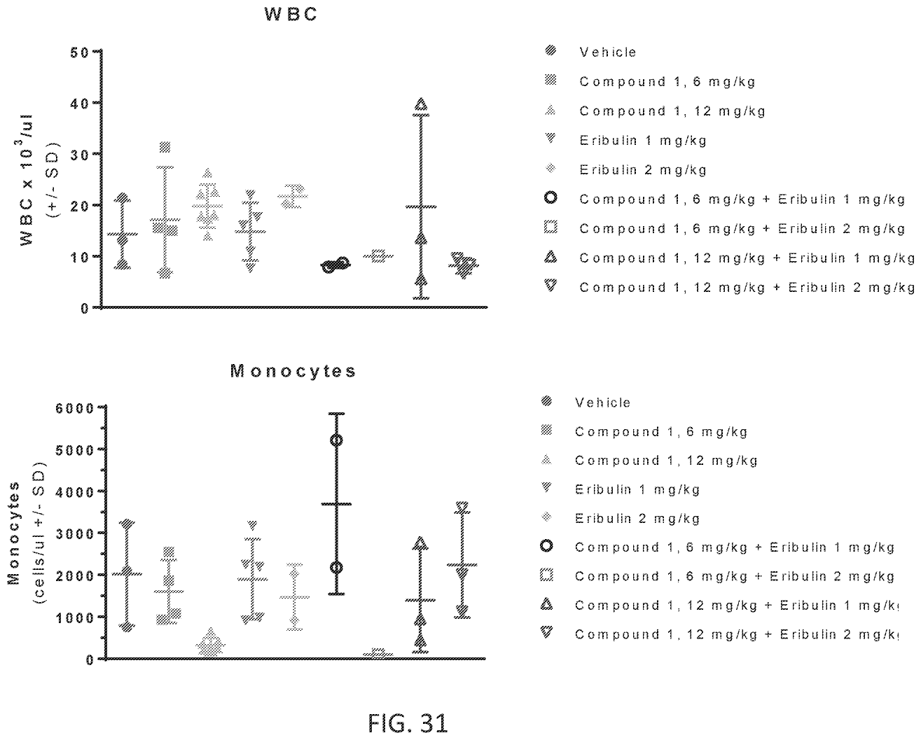

FIG. 31 is a series of graphs showing white blood cell (WBC) and monocyte levels in the various treatment groups.

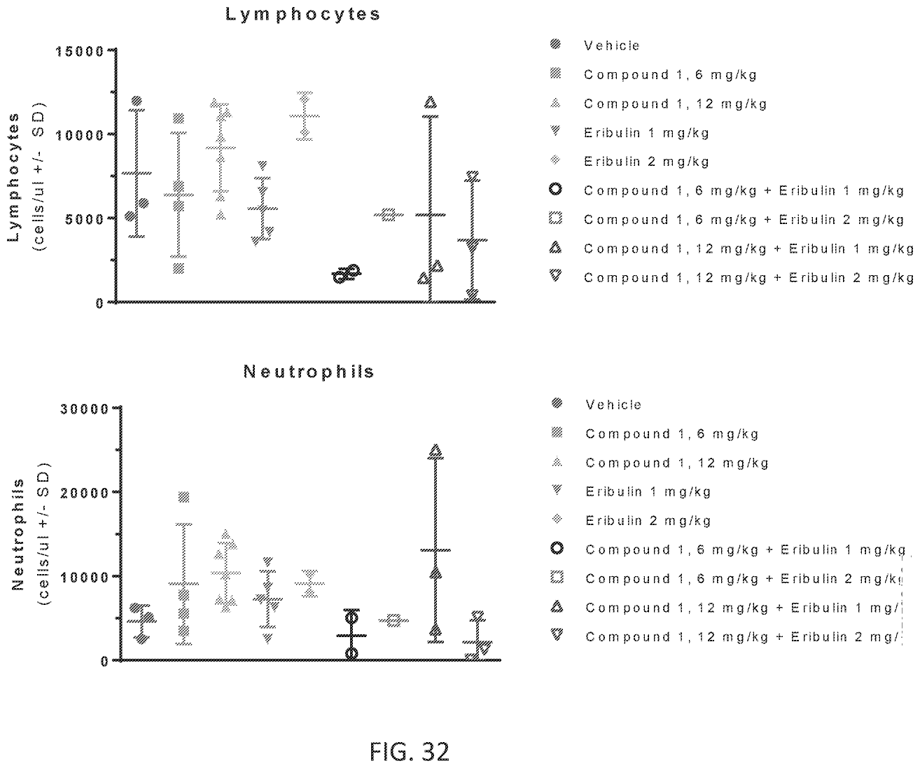

FIG. 32 is a series of graphs showing lymphocyte and neutrophil levels in the various treatment groups.

Without wishing to be bound by theory, the results described in this example demonstrate that the combination of Compound 1 and eribulin can be used to treat cancer, including breast cancer.

### Example 4—Treatment of Breast Cancer Using a Combination of Compound 1 and Inavolisib

The following is a non-limiting example demonstrating the treatment of breast cancer in a mouse model using a combination of Compound 1 and inavolisib.

Animals: Female NU/j mice, 6 weeks of age upon delivery, were received and acclimated at 5 mice per cage for a minimum of 7 days. Mice were housed in autoclaved caging with autoclaved water throughout the course of the study. The animals had free access to food (irradiated chow 2920X) and water. The animals were housed in a temperature and humidity-controlled room with a 12-hour light cycle.

Cell Culture: MCF-7 cells were cultured in Dulbecco's Modified Eagle Medium (DMEM) with 10% FBS.

Tumor Cell Implantation: On the day of implantation, cells were washed 1× with phosphate-buffered saline (PBS). Following washing, cells were pelleted (5 min @1000 rpm, RT) and then counted with a hemocytometer. A cell concentration of 5×10⁶ per mouse was re-suspended in an appropriate amount of PBS and 1:1 Matrigel (kept on ice until implantation). Forty-eight hours prior to cell implantation, a 17-β estradiol pellet (0.36 mg 60-day slow release pellet) was implanted subcutaneously between the scapulae of each mouse. The breast cell line MCF-7 was injected in to the mammary fat pad, which required the use of anesthesia to insure placement of cells in to the mammary fat pad. The mice were anesthetized with a combination of 4% Isoflurane and 2.5/min O2 in an induction chamber. Once deeply anesthetized, the animals were positioned ventral side up and anesthesia maintained through a fitted nose cone. The MCF-7 cells were then injected at a volume of 100 μl containing 5×10⁶ cells per mouse. The injection site was cleansed using alcohol before injection to prevent any infection.

Tumor Assessment: prior to cell implantation, all animals were ear tagged. Beginning 5 days post cell implantation, tumors were measured two times weekly (length×width) using a wireless Mitutoyo UWAVE-T digital calipers in conjunction with UWAVE-R to record measurements. Once the average tumor volume reached approximately 50 mm³ ((length×width²)π/6), animals were randomized by average tumor volume and placed into 8 groups of 15 mice. Randomization was determined by tumor volume of all of the mice. Randomization was performed to ensure that each of the groups have similar tumor averages (approximately 50 mm³).

Treatment: four groups of mice were treated as put forth in Table B. The mice in group 1 were dosed orally (PO) with a vehicle control. Inavolisib was administered orally (PO) once per day (QD) at an amount of 25 mg/kg for the duration of the study. Compound 1 was administered subcutaneously (SC) once every four days (Q4D) at an amount of 8 mg/kg for the duration of the study. All PO and SC dose volumes were calculated based on 10 mb/kg of body weight.

TABLE B

| | | Number | | |
|---|---|---|---|---|
| Group | | of mice | Dose | Dose Route/ |
| # | Group ID | per group | (mg/kg) | Frequency |
| 1 | Vehicle | 15 | NA | PO/QD |
| 2 | Compound 1 | 15 | 8 | SC/Q4D |
| 7 | Compound 1 + | 15 | 8 | SC/Q4D |
| | Inavolosib | | 25 | PO/Q4D |
| 8 | Inavolosib | 15 | 25 | PO/QD |

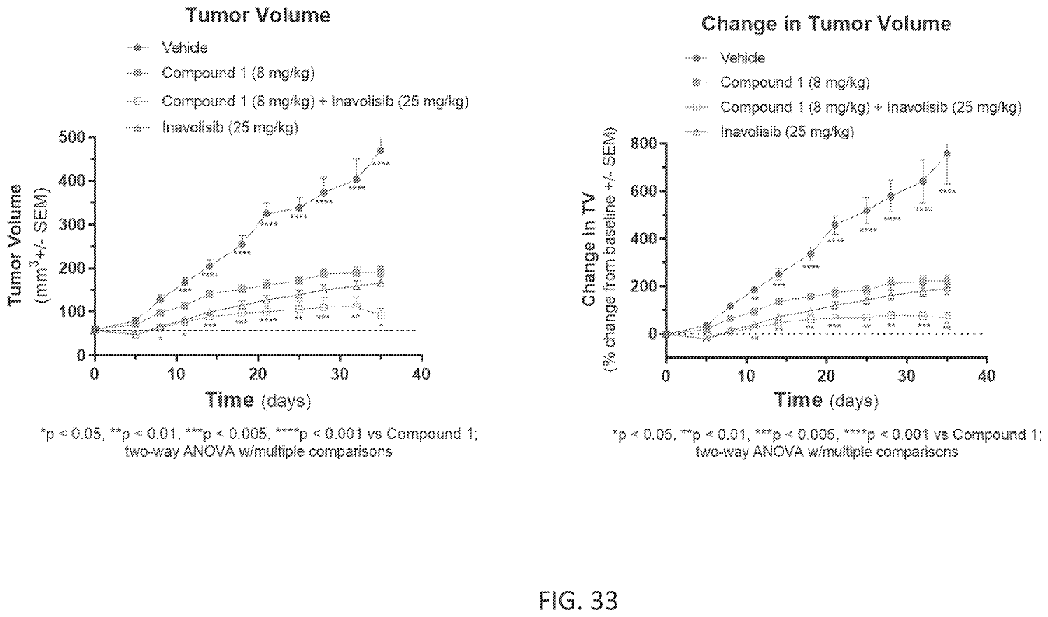

FIG. 33 is a graph of tumor volume (left panel) and change in tumor volume (Tight panel) over the course of the study in the different treatment groups.

Tumor growth inhibition in the various treatment groups on Day 25 of the study is shown in Table C.

| | Starting | Tumor | Tumor |
|---|---|---|---|
| | Tumor | Volume on | Growth |
| | Volume | Day 25 | Inhibition |
| Treatment | (mm³) | (mm³) | (%) |
| Vehicle | 59 | 469 | — |
| Compound 1 (8 mg/kg) | 59 | 191 | 68 |
| Compound 1 (8 mg/kg) + | 59 | 92 | 92 |
| Inavolisib (25 mg/kg) | | | |
| Inavolisib (25 mg/kg) | 59 | 167 | 74 |

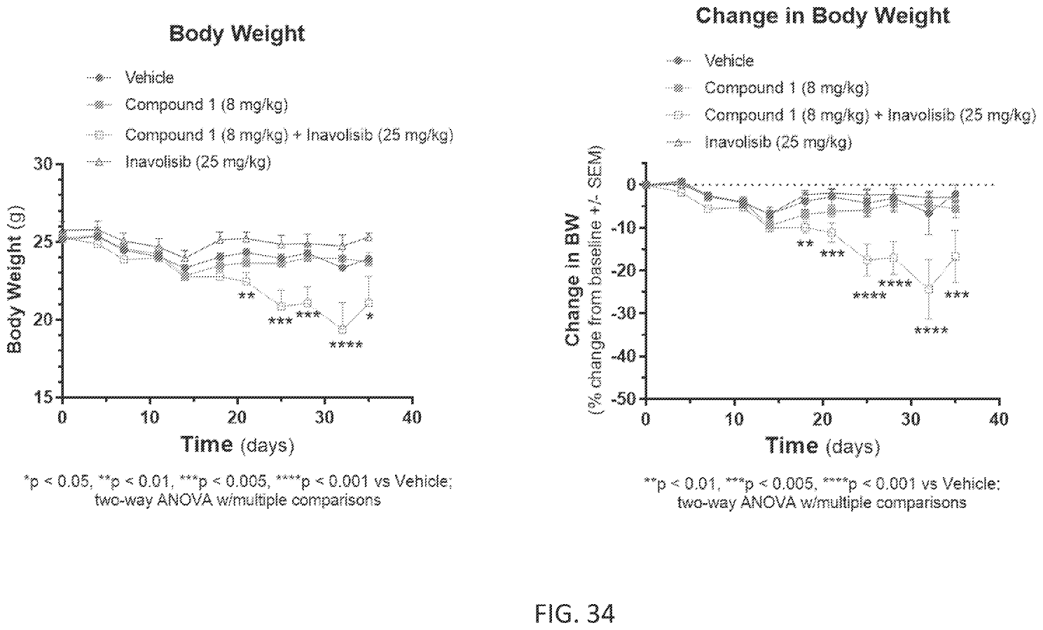

FIG. 34 is a series of graphs showing body weight and change in body weight in the various treatment groups over the course of the study. As shown in FIG. 34, all groups, including the vehicle group, lost weight. Weight loss in the Compound 1 and inavolisib (groups 2-5) was increased as compared to the vehicle group. Weight loss in the combination groups was consistently greater than the vehicle group.

Without wishing to be bound by theory, the results described in this example demonstrate that the combination of Compound 1 and inavolisib can be used to treat cancer, including breast cancer.

What is claimed is:

1. A combination comprising at least one MetAP2 inhibitor, or a pharmaceutically acceptable salt thereof, and eribulin, or a pharmaceutically acceptable salt thereof, for use in treating a cancer in a subject,
wherein the MetAP2 inhibitor is: 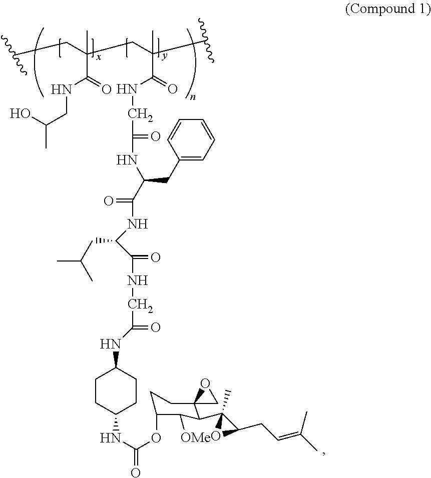
or a pharmaceutically acceptable salt thereof, wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

2. A method of treating cancer in a subject in need thereof, the method comprising administering to the subject at least one therapeutically effective amount of at least one MetAP2 inhibitor, or a pharmaceutically acceptable salt thereof, and at least one therapeutically effective amount of eribulin, or a pharmaceutically acceptable salt thereof,
wherein the MetAP2 inhibitor is: 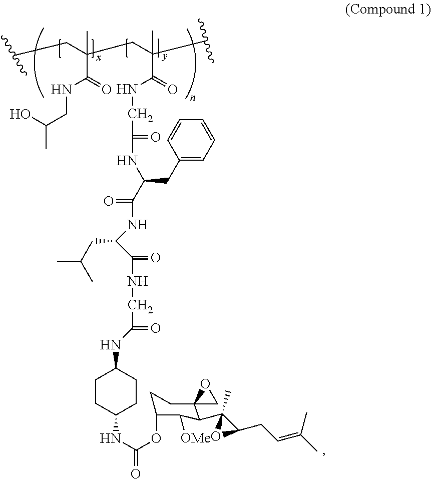
or a pharmaceutically acceptable salt thereof, wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

3. The combination for use or method of any one of the preceding claims, wherein the at least one MetAP2 inhibitor, or pharmaceutically acceptable salt thereof, and the eribulin, or pharmaceutically acceptable salt thereof, are administered concurrently or in temporal proximity.

4. The combination for use or method of any one of the preceding claims, wherein the eribulin is eribulin mesylate.

5. The combination for use or method of any one of the preceding claims, wherein the eribulin is administered/for administration to the subject in an amount of:
i) about 1.4 mg/m²;
ii) about 1.1 mg/m²; or
iii) about 0.7 mg/m², and
wherein the MetAP2 inhibitor, or pharmaceutically acceptable salt thereof, is administered/for administration to the subject in an amount of:
i) about 49 mg/m²;
ii) about 36 mg/m²; or
iii) about 65 mg/m²; or
iv) about 27 mg/m².

6. The combination for use or method of any one of the preceding claims, wherein the MetAP2 inhibitor is administered/for administration:
i) once every 14 days (Q14D);
ii) once every 7 days (Q7D); or
iii) once every 21 days (Q21D).

7. The combination for use or method of any one of the preceding claims, wherein the eribulin is administered/for administration on day 1 and one day between days 5 and 11 (inclusive) of a 21-day cycle, preferably wherein the eribulin or eribulin mesylate is administered/for administration on day 1 and day 8 of a 21-day cycle.

8. The combination for use or method of any one of the preceding claims, wherein the eribulin and the MetAP2 inhibitor, or pharmaceutically acceptable salt thereof, is administered/for administration to the subject in a first 21-day cycle, followed by a second 21-day cycle,
wherein the first 21-day cycle comprises:
i) administering the MetAP2 inhibitor, or pharmaceutically acceptable salt thereof, on days 1 and 15 of the first 21-day cycle; and
ii) administering the eribulin on day 1 and one day between days 5 and 11 (inclusive), preferably day 8, of the first 21-day cycle;
wherein the second 21-day cycle comprises:
i) administering the MetAP2 inhibitor, or pharmaceutically acceptable salt thereof, on day 8 of the second 21-day cycle; and
ii) administering the eribulin on day 1 and one day between days 5 and 11 (inclusive), preferably day 8, of the second 21-day cycle.

9. The combination for use or method of any one of the preceding claims, wherein the eribulin and the MetAP2 inhibitor, or pharmaceutically acceptable salt thereof, is administered/for administration to the subject in 21-day cycle, wherein the 21-day cycle comprises:
i) administering the MetAP2 inhibitor, or pharmaceutically acceptable salt thereof, on days 1 and 8 of the 21-day cycle; and
ii) administering the eribulin on days 1 and 8 of the 21-day cycle;
or
i) administering the MetAP2 inhibitor once during the 21-day cycle; and
ii) administering the eribulin on days 1 and 8 of the 21-day cycle.

10. The combination for use or method of any one of the preceding claims, wherein the subject:
i) has breast cancer, preferably wherein the breast cancer is triple-negative beast cancer, preferably wherein the triple-negative breast cancer is metastatic.
ii) has at least one of:
a) a BMI of greater than or equal to about 30 kg/m²; and
b) an HbA1c level of greater about 5.5%; and/or
iii) has previously received at least one line of treatment for the cancer.

11. A combination comprising at least one MetAP2 inhibitor, or a pharmaceutically acceptable salt thereof, fulvestrant, or a pharmaceutically acceptable salt thereof, and alpelisib, or a pharmaceutically acceptable salt thereof, for use in treating a cancer in a subject,
wherein the MetAP2 inhibitor is: 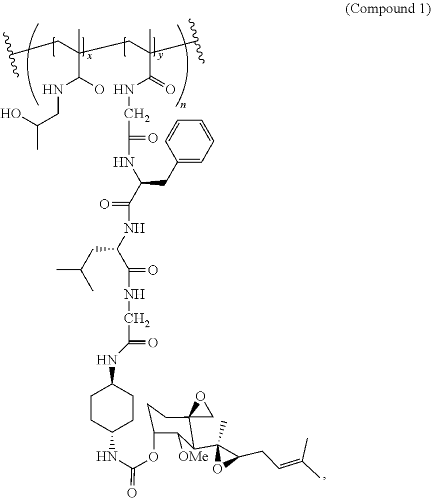
wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

12. A method of treating cancer in a subject in need thereof, the method comprising administering to the subject at least one therapeutically effective amount of at least one MetAP2 inhibitor, or a pharmaceutically acceptable salt thereof, at least one therapeutically effective amount of fulvestrant, or a pharmaceutically acceptable salt thereof, and at least one therapeutically effective amount of alpelisib, or a pharmaceutically acceptable salt thereof,
wherein the MetAP2 inhibitor is: 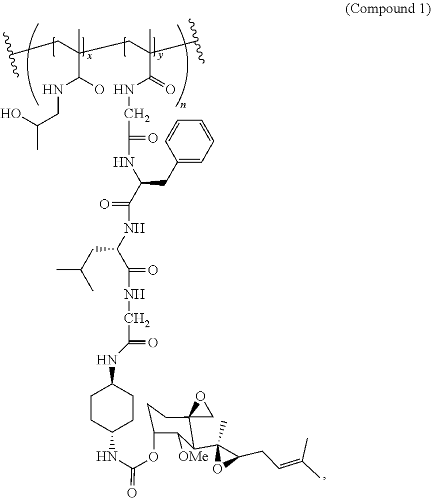
wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

13. The combination for use or the method of any one of the preceding claims, wherein the at least one MetAP2 inhibitor, or pharmaceutically acceptable salt thereof, the alpelisib, or pharmaceutically acceptable salt thereof, and the fulvestrant, or pharmaceutically acceptable salt thereof, are administered concurrently or in temporal proximity.

14. The combination for use or the method of any one of the preceding claims,
wherein the alpelisib, or pharmaceutically acceptable salt thereof, is administered/for administration to the subject in an amount of:
i) about 300 mg;
ii) about 250 mg; or
iii) about 200 mg,
wherein the fulvestrant, or pharmaceutically acceptable salt thereof, is administered/for administration to the subject in an amount of:
i) about 500 mg; or
ii) about 250 mg; and
wherein the MetAP2 inhibitor, or pharmaceutically acceptable salt thereof, is administered/for administration to the subject in an amount of:
i) about 49 mg/m²;
ii) about 36 mg/m²;
iii) about 65 mg/m²; or
iv) about 27 mg/m².

15. The combination for use or the method of any one of the preceding claims,
wherein the MetAP2 inhibitor is administered/for administration once every 14 days (Q14D),
wherein the alpelisib inhibitor is administered/for administration once daily (QD), and
wherein the fulvestrant is administered/for administration once every 14 days (Q14D), preferably wherein following the third administration of fulvestrant, the fulvestrant is administered/for administration once every 28 days (Q28D).

16. The combination for use or the method of any one of the preceding claims, wherein the MetAP2 inhibitor, or pharmaceutically acceptable salt thereof, the alpelisib, or pharmaceutically acceptable salt thereof, and fulvestrant, or pharmaceutically acceptable salt thereof, is administered/for administration to the subject in a first 28-day, followed by a second 21-day cycle,
wherein the first 28-day cycle comprises:
i) administering the MetAP2 inhibitor, or pharmaceutically acceptable salt thereof, on days 1 and 15 of the first 28-day cycle;
ii) administering the fulvestrant, or pharmaceutically acceptable salt thereof, on days 1 and 15 of the first 28-day cycle; and
iii) administering the alpelisib, or pharmaceutically acceptable salt thereof, on each of days 15-28 of the first 28-day cycle;
wherein the second 21-day cycle comprises:
i) administering the MetAP2 inhibitor, or pharmaceutically acceptable salt thereof, on days 1 and 15 of the second 28-day cycle; and
ii) administering the fulvestrant, or pharmaceutically acceptable salt thereof, on day 1 of the second 28-day cycle; and
iii) administering the alpelisib, or pharmaceutically acceptable salt thereof, on each of days 1-28 of the second 28-day cycle.

17. A combination comprising at least one MetAP2 inhibitor, or a pharmaceutically acceptable salt thereof, and inavolisib, or a pharmaceutically acceptable salt thereof, for use in treating a cancer in a subject,
wherein the MetAP2 inhibitor is: 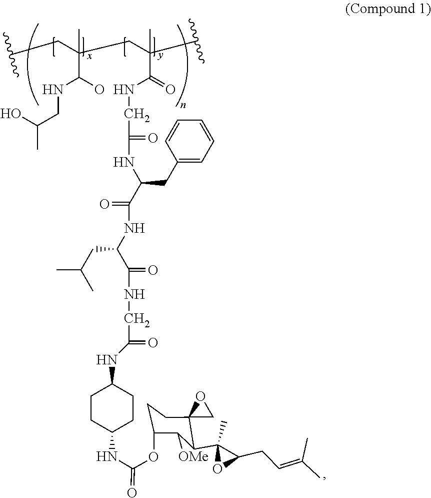
wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

18. A method of treating cancer in a subject in need thereof, the method comprising administering to the subject at least one therapeutically effective amount of at least one MetAP2 inhibitor, or a pharmaceutically acceptable salt thereof, and at least one therapeutically effective amount of inavolisib, or a pharmaceutically acceptable salt thereof,
wherein the MetAP2 inhibitor is: 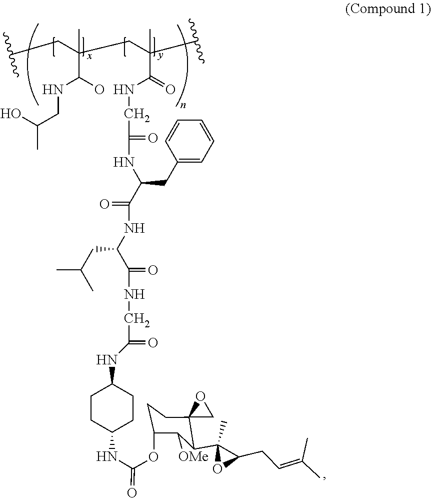
wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

19. The combination for use or the method of any one of the preceding claims,
wherein the inavolisib is administered/for administration to the subject in an amount of:
i) about 3 mg;
ii) about 6 mg;
iii) about 9 mg; or
iv) about 12 mg; and
wherein the MetAP2 inhibitor, or pharmaceutically acceptable salt thereof, is administered/for administration to the subject in an amount of:
i) about 49 mg/m²;
ii) about 36 mg/m²; or
iii) about 65 mg/m².

20. The combination for use or the method of any one of the preceding claims,
wherein the MetAP2 inhibitor is administered/for administration once every 4 days (Q4D); and
wherein the inavolisib is administered/for administration once daily.

21. The combination for use or the method of any one of the preceding claims, wherein the subject has breast cancer, preferably wherein the breast cancer is HR+HER2− breast cancer, preferably wherein the breast cancer is relapsed breast cancer.

22. The combination for use or the method of any one of the preceding claims, preferably wherein the breast cancer is
i) characterized by progressive disease more than about 12 months from the completion of a neoadjuvant and/or adjuvant endocrine therapy;
ii) identified to be progressive while the subject is being treated or after the subject has been treated with at least one endocrine therapy in combination with at least one CDK4/6 inhibitor.

23. The combination for use or the method of any one of the preceding claims, wherein the subject
i) has been previously treated with at least one endocrine therapy in combination with at least one CDK4/6 inhibitor, preferably wherein the subject has been treated with the at least one endocrine therapy in combination with the at least one CDK4/6 inhibitor for at least about 12 months;
ii) hast at least one PIK3CA mutation;
iii) is a postmenopausal woman; and/or
iv) has at least one of:
a) HbA1c levels of about 5.5% to about 6.4%
b) fasting plasma glucose (FPG) level of greater than about 100 mg/dL (5.6 mmol/L) and less than about 140 mg/dL (7.7 mmol/L);
c) a body mass index (BMI) of greater than about 20 kg/m²
d) a Homeostasis Model Assessment of Insulin Resistance (HOMA-IR) score of greater than about 1.8
e) a BMI of greater than or equal to about 30 kg/m²; and
f) an HbA1c level of greater than about 5.5%.

24. The combination for use or the method of any one of the preceding claims, wherein the subject has at least one metabolic dysfunction, wherein the at least one metabolic dysfunction is excessive visceral adiposity, dyslipidemia, obesity (BMI≥30), elevated leptin levels, depressed adiponectin levels, high leptin-to-adiponectin ratio, elevated fasting insulin levels, elevated fasting insulin levels accompanied by chronic inflammation, insulin resistance, high fasting glucose, elevated HbA1c, or any combination thereof.
